(19) United States
(12) Patent Application Publication
(10) Pub. No.: US 2023/0257443 A1
(43) Pub. Date: Aug. 17, 2023

(54) CHIMERIC NKG2D PROTEIN

(71) Applicants: King's College London, Strand, London (GB); Leucid Bio Ltd, Great Cambourne Cambridgeshire (GB)

(72) Inventors: John Maher, Strand, London (GB); David Marc Davies, Strand, London (GB); Fahima Kausar, Great Cambourne Cambridgeshire (GB); Daniel Larcombe-Young, Strand, London (GB)

(21) Appl. No.: 17/927,132

(22) PCT Filed: May 21, 2021
(86) PCT No.: PCT/EP2021/063699

§ 371 Date: Nov. 22, 2022

(30) Foreign Application Priority Data
May 22, 2020 (GB) 2007655.0

(51) Int. Cl. C07K 14/705; C12N 5/0783; C12N 5/0786; A61K 35/15; A61K 35/17; A61P 35/00

(52) U.S. Cl. CPC C07K 14/7056; C12N 5/0646; C12N 5/0636; C12N 5/0645; A61K 35/15; A61K 35/17; A61P 35/00

(57) ABSTRACT

This invention relates to an immunoresponsive cell comprising a chimeric NKG2D protein. The immunoresponsive cell is a T-cell, natural killer (NK) cell, macrophage or neutrophil and the chimeric NKG2D protein comprises a human NKG2D extracellular domain or a variant thereof, and a murine NKG2D transmembrane domain or a variant thereof. The disclosure also relates to isolated polynucleotide(s) encoding the chimeric NKG2D protein and the use of the immunoresponsive cells or isolated polynucleotides in therapy or the treatment of cancer.

### TECHNICAL FIELD

This invention relates to an immunoresponsive cell comprising a chimeric NKG2D protein. The immunoresponsive cell is a T-cell, natural killer (NK) cell, macrophage or neutrophil and the chimeric NKG2D protein comprises a human NKG2D extracellular domain or a variant thereof, and a murine NKG2D transmembrane domain or a variant thereof. The disclosure also relates to isolated polynucleotide(s) encoding the chimeric NKG2D protein and the use of the cells or isolated polynucleotides in therapy or the treatment of cancer.

### BACKGROUND

Immunotherapy using chimeric antigen receptor (CAR)-engineered T-cells has proven transformative in the management of B-cell malignancy and multiple myeloma. However, application of this technology to solid tumour immunotherapy is impeded by the lack of tumour-selective targets. Most tumour antigens are intracellular and thus cannot easily be recognised by CAR T-cells. Consequently, most solid tumour directed CARs that are currently under development engage targets that are upregulated in tumour cells, but which are found at lower levels in normal tissues.

One of the few target groups that exhibits a high degree of tumour selectivity are the NKG2D ligands. In man, these comprise a group of 8 stress-induced proteins (MICA, MICB, ULBP1-6) that are aberrantly expressed on virtually all tumour cell types. Moreover, NKG2D ligands are also found on tumour associated stromal elements such as endothelium, regulatory T-cells and myeloid derived suppressor cells (Parihar, R., et al., 2019, Cancer Immunol. Res. 7(3):363-375; Schmiedel & Mandelboim, 2018, Front. Immunol. (9)2040). Mice that are genetically deficient in NKG2D demonstrate impaired immunosurveillance for both epithelial and lymphoid malignancies. Evidence that NKG2D ligands are safe therapeutic targets is supported by the fact that they are not found in healthy tissues.

The NKG2D receptor is naturally expressed by natural killer (NK) and some T-cell populations. In humans, NKG2D associates exclusively with the adaptor protein DNAX-activating protein 10 (DAP10), which mediates co-stimulatory signalling though the presence of a YxxM motif within its intracellular domain (Wu, J. et al., J Exp Med 192, 1059-1068 (2000)). However, since DAP10 lacks an immunoreceptor tyrosine-based activation motif (ITAM), NKG2D engagement does not lead to full T-cell activation.

To provide an activating signal, several NKG2D-based CARs have been generated in which an ITAM-containing motif (typically from CD3ζ) is fused in frame with the endodomain of NKG2D, either with or without an additional co-stimulatory motif.

There remains a need for highly selective CARs with anti-tumour efficacy. The present invention seeks to address one or more of the aforementioned issues.

### SUMMARY OF THE INVENTION

We have engineered a chimeric NKG2D protein comprising a human NKG2D extracellular domain and a murine NKG2D transmembrane domain, which allows association with DAP10 and/or DAP12 to form a compact adaptor-based CAR which drives full T-cell activation.

Thus, the disclosure provides an immunoresponsive cell comprising a chimeric NKG2D protein. The immunoresponsive cell is a T-cell, natural killer (NK) cell, macrophage, or neutrophil. The chimeric NKG2D protein comprises a human NKG2D extracellular domain or a variant thereof and a murine NKG2D transmembrane domain or a variant thereof.

The disclosure also provides an isolated polynucleotide(s) encoding the chimeric NKG2D protein of the disclosure. Also disclosed are vectors, optionally expression vectors comprising the isolated polynucleotide(s) of the disclosure. The disclosure also relates to host cells comprising the polynucleotide(s) or vector(s) of the disclosure. In addition, the disclosure relates to a pharmaceutical composition comprising the immunoresponsive cell(s), isolated polynucleotide(s), vector(s), or host cell(s) of the disclosure.

The disclosure also provides a kit comprising the immunoresponsive cell(s), the isolated polynucleotide(s), vector(s), pharmaceutical composition, or host cell(s) of the disclosure.

Also disclosed is a method of preparing an immunoresponsive cell according to the disclosure, the method comprising introducing the isolated polynucleotide(s) or vector(s) of the disclosure into a T-cell, natural killer (NK) cell, macrophage, or neutrophil.

The disclosure also relates to the immunoresponsive cell(s), isolated polynucleotide(s), vector(s), pharmaceutical composition, or host cell(s) of the disclosure for use in (i) therapy or (ii) the treatment of cancer. Also disclosed is a method for directing an immune response to a target cell in a subject in need thereof, wherein the method comprises administering to the subject the immunoresponsive cell(s), the isolated polynucleotide(s), the vector(s), pharmaceutical composition or the host cell(s) of the disclosure.

The disclosure also relates to a method of treating cancer, wherein the method comprises administering to a subject suspected of having or having cancer, the immunoresponsive cell(s), isolated polynucleotide(s), vector(s), pharmaceutical composition or host cell(s) of the disclosure.

### BRIEF DESCRIPTION OF THE DRAWINGS

One or more embodiments of the invention will now be described, by way of example only, with reference to the accompanying drawings, in which:

FIG. 1 is a schematic demonstrating the structure of each of the constructs that have been generated. Nomenclature is indicated above each chimeric receptor. To provide an additional control, NKG2D was over-expressed alone in indicated T-cell cultures.

FIG. 2 shows the expression of the CARs of FIG. 1 in human CD4⁺ T-cells. Cell surface expression (percentage transduction) of the CARs was demonstrated using flow cytometry following the staining of engineered human CD4⁺ T-cells with antibodies directed against NKG2D, making comparison with untransduced CD4⁺ T-cells (FIG. 2A, left graph). Transduction efficiency was determined in CD4⁺ T-cells since, unlike CD8⁺ T-cells, they do not express endogenous cell surface NKG2D. The right graph of FIG. 2A shows the mean fluorescence intensity (MFI) of transduced CD4⁺ T-cells, making comparison with untransduced (UT) CD4⁺ T-cells. FIG. 2B shows the percentage transduction of CD4⁺ T-cells across multiple donors for the N1012, N5, CYAD-01, CYAD-01_10 and NKG2D constructs. CYAD-01 is an NKG2D-targeted CAR T-cell which is currently undergoing clinical development by Celyad Oncology and consists of a fusion of NKG2D to the intracellular domain of CD3 (Zhang et al, 2005, Blood 106:1544-1551). In the CYAD-01_10 CAR, additional DAP10 has been stoichiometrically co-expressed with CYAD-01 using a ribosomal skip peptide. The CYAD-01 and CYAD-01_10 CARs are thus comparator CARs.

FIG. 3 shows the percentage viability of tumour cells following co-culture with T-cells according to FIG. 1. T-cells were co-cultured with either pancreatic cancer (BxPC3_LT), head and neck cancer (HN3_LUC), or malignant mesothelioma (Ju77) cells at effector: target ratios ranging from 1:1-1:64 for 72 hours. Tumour cell viability was assessed after the 72 hours using an MTT assay and is expressed as a percentage of that observed in the absence of T-cell co-culture.

FIG. 4 shows the viability of various firefly luciferase (ffLUC)-tagged tumour cells following repeated in vitro co-culture with N5 and the indicated other CAR T-cells. FIG. 4A shows the viability of ffLUC-tagged BXPC3 tumour cells following repeated in vitro stimulation by N1 to N5 CAR CD4⁺ T-cells (left panels). The viability of Ju77, Ren and OVSAHO tumour cells are shown in FIGS. 4B and 4C (left panels). The levels of proliferation of the N1 to N5 CAR CD4+ and/or other CAR T-cells following each round of stimulation with the ffLUC-tagged BXPC3, Ju77, Ren and OVSAHO tumour cells is also shown (right panels). The data of FIG. 4A are presented from three independent donors. FIG. 4B also shows results from N1012 CAR T-cells, T-cells engineered to express NKG2D alone or untransduced T-cells, while FIG. 4C shows results from the CYAD-01 or untransduced T-cell groups, as compared to N5 CAR T-cells. FIG. 4D presents graphs showing the total number of restimulations achieved by N1012, N5 or CYAD-01, NKG2D CAR T-cells when incubated with Ren, BxPC3_LT or Ju77 tumour cells, making comparison with T-cells that over-express NKG2D alone or untransduced T-cells; each graph point represents cells from a different donor. FIG. 4E shows the maximum fold expansion achieved by N1012, N5 or CYAD-01 CAR T-cells when restimulated with Ren, BxPC3_LT and Ju77 tumour cells, making comparison with T-cells that over-express NKG2D alone or untransduced T-cells. FIG. 4F presents the expression profile of the T-cell exhaustion markers, PD-1, LAG-3 and TIM-3 in the indicated CAR T-cell groups prior to and after stimulation with tumour cells.

FIG. 5A shows the levels of IFN-γ secreted by the CD4⁺ T-cells during co-culture with BxPC3, HN3_LUC or Ju77 tumour cells at a 1:1 CAR T-cell: target ratio. Co-culture supernatants were removed after 72 hours and assessed for cytokine presence by enzyme-linked immunosorbent assay (ELISA). FIG. 5B shows the fold increase in IFN-γ secretion by the CAR T-cells during co-culture with BxPC3, Ren or Ju77 tumour cells across multiple donors, when compared to untransduced T-cells and T-cells that had been engineered to express NKG2D alone.

FIG. 6 shows the in vitro fold expansion of the indicated CAR T-cells when cultured for 14 days in IL-2-containing medium; the T-cells were subsequently used in in vivo experiments.

FIG. 7 shows the growth of ffLUC-tagged BxPC3 cells in vivo in NSG mice. The introduction of CAR CD4⁺ T-cells by i.p. injection into the mice is shown as a dashed line on the graphs. Tumour growth was monitored weekly by bioluminescence imaging and the data are presented as both average total flux (photons/second) per treatment group (FIG. 7A), and total flux (photons/second) per individual mouse (FIG. 7B). FIG. 7C shows the experiment of FIG. 7B extended to 70 days.

FIG. 8 shows the weight of the NSG mice during the experiment shown in FIG. 7B. Weight was monitored weekly. Data are presented as a percentage of original weight.

FIG. 9 shows the growth of ffLUC-tagged BxPC3 cells in vivo in SCID-Beige mice. The introduction of CAR CD4⁺ T-cells by i.p. injection into the mice is shown as a dashed line on the graphs. Tumour growth was monitored weekly by bioluminescence imaging and the data are presented as total flux (photons/second) per individual mouse.

FIG. 10 shows serial bioluminescence emission from individual mice following tumour re-challenge on day 63. Individual mice were selected from FIG. 9 that remained tumour-free.

FIG. 11 shows the growth of ffLUC-tagged SKOV3 cells in vivo in SCID-Beige mice. N1012 or N5 CAR CD4⁺ T-cells, or a PBS control, were introduced into the mice by i.p. injection, shown as a dashed line on the graphs. Tumour growth was monitored weekly by bioluminescence imaging and the data are presented as average total flux (photons/second) per treatment group (FIG. 11A) or total flux (photons/second) per individual mouse in FIG. 11B.

FIG. 12 shows the weight of the SCID-Beige mice during the experiment shown in FIG. 11. Weight was monitored weekly. Data are presented as a percentage of original weight.

FIG. 13 shows the growth of ffLUC-tagged H226 malignant mesothelioma cells in vivo in NSG mice. N1012, NKG2D, N5 CAR CD4⁺ T-cells, or a PBS control, were introduced into the mice by i.p. injection, shown as a dashed line on the graphs. Tumour growth was monitored weekly by bioluminescence imaging and the data are presented as average total flux (photons/second) per treatment group (FIG. 13A) or total flux (photons/second) per individual mouse in FIG. 13B.

FIG. 14 shows the intravenous administration (shown as a dashed line on the graph of FIG. 14A) of 1×10⁷ N5, NKG2D, CYAD-01, N1012 CAR T-cells or PBS into NSG mice engrafted with a mesothelioma patient derived xenograft (PDX) tumour. Tumour growth was determined by weekly caliper measurements, with all data presented as tumour volume (mm³). Mice were monitored closely and weighed three times per week for signs of ill health. The data are presented as average tumour volume (left graph, FIG. 14A), the average percentage tumour volume change (right graph, FIG. 14A), tumour volume per mouse (FIG. 14B) and the probability of survival per treatment group (FIG. 14C).

FIG. 15 shows results from a comparable experiment to FIG. 14, except that a lower T cell dose was administered (4×10⁶ T-cells). In addition, CAR T-cells that expressed CYAD-01_10 were tested. Tumour growth was determined by weekly caliper measurements, with all data presented as tumour volume (mm³). Mice were monitored closely and weighed three times per week for signs of ill health. The data are presented as average tumour volume (FIG. 15A), tumour volume per mouse (FIG. 15B) and the probability of survival per treatment group (FIG. 15C).

### DETAILED DESCRIPTION

Chimeric NKG2D Protein

As noted above, the disclosure provides an immunoresponsive cell comprising a chimeric NKG2D protein. The immunoresponsive cell is a T-cell, natural killer (NK) cell, macrophage, or neutrophil. The chimeric NKG2D protein comprises a human NKG2D extracellular domain or a variant thereof and a murine NKG2D transmembrane domain or a variant thereof.

In the context of the present invention, the term “chimeric NKG2D protein” refers to a NKG2D protein which is formed of domains from two or more different organisms. For the present disclosure, this refers to domains from at least human and murine sources.

In an embodiment, the chimeric NKG2D protein is a human-murine chimeric protein. Murine may be selected from rat, mouse, and combinations thereof. Thus, the chimeric NKG2D protein may be human-mouse. Alternatively, the chimeric NKG2D protein may be human-rat, or human, rat and mouse. In an embodiment, the chimeric NKG2D protein is heterochimeric. “Heterochimeric” will be understood to refer to a composition derived from two different organisms. Thus, in the context of a chimeric NKG2D protein, a heterochimeric NKG2D protein is restricted to murine and human domains, for example human and mouse domains, or human and rat domains.

In an embodiment, the chimeric NKG2D protein comprises from N terminus to C terminus the murine NKG2D transmembrane domain or a variant thereof and a human NKG2D extracellular domain or a variant thereof.

Murine NKG2D Transmembrane Domain and Variant Thereof

Wild-type mouse NKG2D is encoded by the amino acid sequence having UniProt accession no: 054709 (SEQ ID NO:1). The first 66 amino acids are considered to be the intracellular domain, amino acids 67-89 the transmembrane domain, and amino acids 90-232 the extracellular domain.

In one embodiment, the murine NKG2D transmembrane domain is a mouse NKG2D transmembrane domain.

An exemplary mouse NKG2D transmembrane domain sequence is SEQ ID NO:2. SEQ ID NO:2 may otherwise be identified as amino acids 67-89 of UniProt accession no: 054709.

The inventors have found that chimeric NKG2D proteins that comprise this NKG2D transmembrane domain have high levels of surface expression on the immunoresponsive cell. In an embodiment, the mouse NKG2D transmembrane domain comprises or consists of SEQ ID NO:2.

Other mouse NKG2D transmembrane domains are envisaged. For example, the mouse NKG2D transmembrane domain (such as, for example, SEQ ID NO:2) may further comprise a portion of a mouse NKG2D extracellular domain, and optionally a portion of a mouse NKG2D intracellular domain. The portion of the mouse NKG2D extracellular domain may be at the N terminus of the mouse NKG2D transmembrane domain. The portion of the mouse NKG2D intracellular domain may be at the C terminus of the mouse NKG2D transmembrane domain. Alternatively, the portion of the mouse NKG2D extracellular domain may be at the C terminus of the mouse NKG2D transmembrane domain. The portion of the mouse NKG2D intracellular domain may be at the N terminus of the mouse NKG2D transmembrane domain. By “portion”, this may be 1, 2, 3, 4, 6, 7, 8, 9 or 10 amino acids. Each portion may be at least 5, and no more than 10 amino acids. In an embodiment, the portion of the mouse NKG2D extracellular domain may be 6 amino acids. In an embodiment, the portion of the mouse NKG2D intracellular domain may be 10 amino acids. An exemplary mouse NKG2D transmembrane domain, which comprises SEQ ID NO: 2, a portion of a mouse NKG2D extracellular domain and a portion of a mouse NKG2D intracellular domain is SEQ ID NO:3. SEQ ID NO:3 represents amino acids 61-97 of UniProt accession no: 054709 (Rosen et al, 2004, J Immunol 173: 2470-2478). In one embodiment, the mouse NKG2D transmembrane domain comprises or consists of SEQ ID NO:3.

Rat NKG2D transmembrane domains are also suitable transmembrane domains for the present invention. Thus, in an embodiment, the murine NKG2D transmembrane domain is a rat NKG2D transmembrane domain. Wild-type rat NKG2D is encoded by the amino acid sequence having UniProt accession no: 070215 (SEQ ID NO:4). The first 51 amino acids are considered to be the intracellular domain, amino acids 52-74 the transmembrane domain, and amino acids 75-215 the extracellular domain.

One example of a rat NKG2D transmembrane domain is SEQ ID NO:5, which corresponds to amino acids 52-74 of UniProt accession no: 070215. The rat NKG2D transmembrane domain may comprise or consist of SEQ ID NO:5.

Variants of the murine NKG2D transmembrane domain are also envisaged. Variants will be understood to be functional variants, in that the variant will substantially retain the functional activity of the wild type NKG2D murine transmembrane domain, or even improve the functional activity. In one embodiment, the activity is measured using functional assays, such as MTT and measuring cytokine secretion by ELISA.

A variant of the murine NKG2D transmembrane domain may have at least 70%, 75%, 80%, 85%, 90%, 95%, 97%, or 99% sequence identity to the murine NKG2D transmembrane domain, for example to a mouse NKG2D transmembrane domain (such as SEQ ID NO:2 or SEQ ID NO:3) or to a rat NKG2D transmembrane domain (such as SEQ ID NO:5). A variant may have at least 90%, optionally at least 95% sequence identity to a mouse NKG2D transmembrane domain or a rat NKG2D transmembrane domain. Alternatively, a variant murine NKG2D transmembrane domain may comprise a peptide comprising one or more point (i.e. 1, 2, 3, 4, 5, 6, 7, 8, 9, 10 or more) mutations that add, delete or substitute any of the amino acids compared to SEQ ID NO:2, SEQ ID NO:3 or SEQ ID NO:5.

Human NKG2D Extracellular Domain and Variant Thereof

The chimeric NKG2D protein comprises a human NKG2D extracellular domain or a variant thereof.

Wild-type human NKG2D is encoded by the amino acid sequence having UniProt accession no: P26718 (SEQ ID NO:6). Exemplary human NKG2D extracellular domains include, but are not necessarily limited to SEQ ID NO:7 and SEQ ID NO:8. SEQ ID NO:8 comprises SEQ ID NO:7, with the additional 9 amino acid sequence IWSAVFLNS (SEQ ID NO:9) at the N terminus.

Another exemplary human NKG2D extracellular domain is SEQ ID NO: 10. SEQ ID NO: 10 corresponds to SEQ ID NO:7, except that the eight most N-terminal amino acids have been removed in SEQ ID NO:10, as compared to SEQ ID NO:7.

Thus, in one embodiment, the human NKG2D extracellular domain comprises or consists of SEQ ID NO:10. Alternatively, the human NKG2D extracellular domain may comprise or consist of SEQ ID NO:7. In another embodiment, the human NKG2D extracellular domain comprises or consists of SEQ ID NO:8.

Variants of the human NKG2D extracellular domain are also envisaged. Variants will be understood to be functional variants, in that the variant will substantially retain the functional activity of the wild type human NKG2D extracellular domain, or even improve the functional activity. The activity may be measured using functional assays, such as MTT and measuring cytokine secretion by ELISA.

A variant of the human NKG2D extracellular domain may have at least 70%, 75%, 80%, 85%, 90%, 95%, 97% or 99% sequence identity to the human NKG2D extracellular domain, for example SEQ ID NO:7, SEQ ID NO:8 or SEQ ID NO:10. The variant may have at least 90% or at least 95% sequence identity to the human NKG2D extracellular domain. In an embodiment, the variant comprises a peptide comprising one or more point (i.e. 1, 2, 3, 4, 5, 6, 7, 8, 9, 10 or more) mutations that add, delete or substitute any of the amino acids compared to SEQ ID NO:7, SEQ ID NO:8 or SEQ ID NO:10. The one or more point mutations may be one or more point (i.e. 1, 2, 3, 4, 5, 6, 7, 8, 9, 10 or more) deletions of any of the amino acids compared to SEQ ID NO:7. Alternatively, the one or more point mutations may be one or more point (i.e. 1, 2, 3, 4, 5, 6, 7, 8, 9, 10 or more) deletions of any of the amino acids compared to SEQ ID NO:8. Optionally, the one or more point mutations are one or more point (i.e. 1, 2, 3, 4, 5, 6, 7, 8, 9, 10 or more) deletions of any of the amino acids compared to SEQ ID NO:7 and SEQ ID NO:8. In an embodiment, the one or more point deletions are at the N terminus of SEQ ID NO:7. Alternatively, the one or more point deletions are at the N terminus of SEQ ID NO:8. Such deletions at the N terminus of the human NKG2D extracellular domain result in a variant which is a truncated human NKG2D extracellular domain.

Intracellular NKG2D Domain and Variant Thereof

In an embodiment, the chimeric NKG2D protein further comprises an intracellular NKG2D domain or a variant thereof. Optionally, the intracellular NKG2D domain or a variant thereof is in a N terminus of the murine NKG2D transmembrane domain. In an embodiment, the intracellular NKG2D domain or a variant thereof is N-terminal to the murine NKG2D transmembrane domain. In an embodiment the intracellular NKG2D domain or a variant thereof is located at the N-terminus of the chimeric NKG2D protein.

The intracellular NKG2D domain may be a human NKG2D intracellular domain.

An exemplary human NKG2D intracellular domain is SEQ ID NO:11. Another exemplary human NKG2D intracellular domain is SEQ ID NO:12. SEQ ID NO:12 corresponds to SEQ ID NO:11, except that the last amino acid at the C-terminus has been removed. The human NKG2D intracellular domain may comprise or consist of SEQ ID NO:11. In an embodiment, the human NKG2D intracellular domain comprises or consists of SEQ ID NO: 12.

Alternatively, the intracellular NKG2D domain may be a murine NKG2D intracellular domain. The murine NKG2D intracellular domain comprises or consists of a short isoform murine NKG2D intracellular domain.

The murine NKG2D intracellular domain may be a mouse NKG2D intracellular domain.

Exemplary short isoform mouse NKG2D intracellular domains include, but are not necessarily limited to SEQ ID NO:13 and SEQ ID NO:14. In one embodiment, the intracellular NKG2D domain comprises or consists of SEQ ID NO: 13. In another embodiment, the intracellular NKG2D domain comprises or consists of SEQ ID NO:14.

Alternatively, the murine NKG2D intracellular domain may be a rat NKG2D intracellular domain. For example, the rat NKG2D intracellular domain may comprise or consist of SEQ ID NO:15. SEQ ID NO:15 corresponds to amino acids 1-51 of UniProt accession number: 070215 (SEQ ID NO:4).

Variants of the intracellular NKG2D domain are also envisaged. Variants will be understood to be functional variants, in that the variant will substantially retain the functional activity of the wild-type intracellular NKG2D domain on which the variant is based, or even improve the functional activity. The activity may be measured using functional assays, such as MTT and measuring cytokine secretion by ELISA.

A variant may have at least at least 70%, 75%, 80%, 85%, 90%, 95%, 97% or 99% sequence identity to the intracellular NKG2D domain, for example a human (such as SEQ ID NO:11 or SEQ ID NO:12), mouse (such as SEQ ID NO:13 or SEQ ID NO:14) or rat (such as SEQ ID NO: 15) intracellular NKG2D domain. A variant may have at least 90% or at least 95% sequence identity to the intracellular NKG2D domain.

In an embodiment, a variant comprises a peptide comprising one or more point (i.e. 1, 2, 3, 4, 5, 6, 7, 8, 9, 10 or more) mutations that add, delete or substitute any of the amino acids compared to any of SEQ ID NOs:11-15.

In an embodiment, the chimeric NKG2D protein does not comprise a mouse NKG2D intracellular domain. The chimeric NKG2D protein may not comprise a murine NKG2D intracellular domain. Optionally, the chimeric NKG2D protein does not comprise an NKG2D intracellular domain. In another embodiment, the chimeric NKG2D protein is not a protein disclosed in CN 105907783.

Polypeptides that Associate with Chimeric NKG2D Protein of the Invention

The chimeric NKG2D protein of the disclosure may associate with other polypeptides to form a complex, such as a hexamer complex, described herein. Such association may be due to electrostatic forces, such as provided by complementary charged amino acids. Alternatively, or in addition to, such association may be due to hydrogen bonding, or due to hydrophobic interactions. The other polypeptide(s) may comprise or consist of fusion polypeptides as described herein.

In one embodiment, such other polypeptides may be genetically encoded as part of a contiguous chimeric construct with the gene that encodes for the chimeric NKG2D protein of the disclosure. The chimeric NKG2D protein and other polypeptide may then be separated during translation (e.g. using a ribosomal skip peptide or an internal ribosomal entry system) or by post translation cleavage (e.g. using a furin cleavage site). Alternatively, the chimeric NKG2D protein and other polypeptide may remain fused to one another. The chimeric NKG2D protein and other polypeptide may therefore be joined by an optional linker. Such a linker may comprise a cleavage site to facilitate cleavage.

In another embodiment, the chimeric NKG2D protein may be genetically encoded on one construct, and the other polypeptide(s) encoded on one or more further constructs.

DAP12 Polypeptide and Variant Thereof

One example of such a polypeptide that may associate with a chimeric NKG2D protein of the disclosure is the DNAX-activating protein 12 (DAP12) polypeptide. Thus, in one embodiment, the immunoresponsive cell further comprises at least one DNAX-activating protein 12 (DAP12) polypeptide or a variant thereof.

DAP12 may be endogenously expressed in certain organisms and certain cell types. In an embodiment, the DAP12 polypeptide or variant thereof is endogenous. Alternatively, the DAP12 polypeptide or variant thereof may be exogenous.

By endogenous, this will be understood to refer to a polypeptide already within the immunoresponsive cell; i.e. the polypeptide is native to the cell.

As will be appreciated by the skilled person, exogenous refers to a polypeptide originating from outside of the immunoresponsive cell and has thereby been introduced into the cell. The term exogenous thus encompasses native peptide sequences which have been introduced into the cell (thereby increasing the number of native peptides in the cell), or sequences which are not naturally found in the cell.

The DAP12 polypeptide may be mammalian, for example, murine (such as mouse or rat) or human. In an embodiment, the DAP12 polypeptide is human. Wild-type human DAP12 has the amino acid sequence having UniProt accession no: 043914 (SEQ ID NO:16). The first 21 amino acids are considered to be a signal/leader sequence, amino acids 22-40 the extracellular domain, amino acids 41-61 the transmembrane domain, and amino acids 62-113 the cytoplasmic/intracellular domain. The DAP12 polypeptide may comprise or consist of SEQ ID NO:16.

Truncated versions of a DAP12 polypeptide may also be used as a DAP12 polypeptide of the disclosure. For example, the DAP12 polypeptide may comprise or consist of a truncated version of DAP12 comprising only amino acids 62-113 of SEQ ID NO:16 (i.e. the intracellular domain). Such a sequence is referred to as SEQ ID NO: 17.

Other truncated versions may comprise amino acids 41-61 of SEQ ID NO: 16, such a sequence comprising merely the transmembrane domain of human DAP12, and referred to here as SEQ ID NO:18. Thus, in an embodiment, the DAP12 polypeptide comprises or consists of SEQ ID NO:18.

Another truncated version may comprise amino acids 22-61 of SEQ ID NO: 16 (i.e. the extracellular and transmembrane domains), referred to as SEQ ID NO:19 herein. In an embodiment, the DAP12 polypeptide may comprise or consist of SEQ ID NO:19.

Another truncated version of the DAP12 polypeptide suitable for use in the disclosure is SEQ ID NO:20. SEQ ID NO:20 comprises only amino acids 22-113 of SEQ ID NO:16 (i.e. lacking amino acids 1-21, the signal/leader sequence). In an embodiment, the DAP12 polypeptide comprises or consists of SEQ ID NO:20.

In another embodiment, the DAP12 polypeptide comprises SEQ ID NO:47. SEQ ID NO:47 comprises a human DAP12 transmembrane domain and a human DAP12 intracellular domain (amino acids 41-113 of UniProt accession no: 043914). The DAP12 polypeptide, such as SEQ ID NO:47, may further comprise an extracellular domain peptide sequence. For example, the DAP12 polypeptide, such as SEQ ID NO:47, may further comprise a human DAP12 extracellular domain peptide sequence.

The DAP12 polypeptide may be a murine polypeptide, optionally a mouse polypeptide. Wild-type mouse DAP12 has the amino acid sequence having UniProt accession no: 054885 (SEQ ID NO:21). The first 21 amino acids are considered to be a signal/leader sequence, amino acids 22-42 the extracellular domain, amino acids 43-63 the transmembrane domain, and amino acids 64-114 the cytoplasmic/intracellular domain. The DAP12 polypeptide may comprise or consist of SEQ ID NO:21.

Truncated versions of a murine DAP12 polypeptide may also be used as a DAP12 polypeptide of the disclosure. Thus, the DAP12 polypeptide may comprise or consist of a truncated version of DAP12 comprising only amino acids 64-114 of SEQ ID NO:21 (i.e. the intracellular domain). Such a sequence is referred to SEQ ID NO:22.

In another embodiment, the DAP12 polypeptide may comprise or consist of amino acids 43-63 of SEQ ID NO:21. Such a sequence is referred to as SEQ ID NO:23.

Another truncated version of the DAP12 polypeptide of the disclosure is SEQ ID NO:24. SEQ ID NO:24 comprises the murine extracellular (aa 22-42) and murine transmembrane DAP12 (aa 43-63) regions. Thus, in an embodiment the DAP12 polypeptide comprises or consists of SEQ ID NO:24.

A further exemplary truncated version of murine DAP12 is amino acids 22-114 of SEQ ID NO:21. Such a sequence comprises the murine extracellular, transmembrane and intracellular DAP12 domains, and is referred to herein as SEQ ID NO:25. Thus, in an embodiment, the DAP12 polypeptide comprises or consists of SEQ ID NO:25.

It will be appreciated that DAP12 is a homodimer. Thus, typically, the immunoresponsive cell comprises a DAP12 homodimer comprising two DAP12 polypeptides according to the disclosure. In an embodiment, the immunoresponsive cell comprises a DAP12 heterodimer, each peptide of the DAP12 heterodimer comprising a different DAP12 polypeptide of the disclosure. At least one of the DAP12 polypeptides of the heterodimer may comprise a DAP12 fusion polypeptide as described herein.

Variants of the DAP12 polypeptide are also envisaged. Variants will be understood to be functional variants, in that the variant will substantially retain the functional activity of the DAP12 polypeptide on which the variant is based, or even improve the functional activity.

The activity may be measured using functional assays, such as MTT and measuring cytokine secretion by ELISA.

A variant may have at least 70%, 75%, 80%, 85%, 90%, 95%, 97% or 99% sequence identity to the DAP12 polypeptide, for example to a human DAP12 polypeptide (such as any of SEQ ID NO:16, 17, 18, 19, 20 and 47) or to a mouse DAP12 polypeptide (such as any of SEQ ID NO:21, 22, 23, 24 and 25).

The variant may comprise a peptide comprising one or more (i.e. 1, 2, 3, 4, 5, 6, 7, 8, 9, 10 or more) point mutations that add, delete or substitute any of the amino acids of the amino acids of DAP12 (such as that of wild-type human DAP12 (e.g. SEQ ID NO: 16, 17, 18, 19, 20 or 47) or wild-type mouse DAP12 (e.g. SEQ ID NO:21, 22, 23, 24 or 25).

DAP10 Polypeptide and Variant Thereof

Another example of a polypeptide that may associate with a chimeric NKG2D protein of the disclosure is the DNAX-activating protein 10 (DAP10) polypeptide. In an embodiment, the immunoresponsive cell further comprises at least one DNAX-activating protein 10 (DAP10) polypeptide or variant thereof.

DAP10 may be endogenously expressed in certain organisms and certain cell types. In an embodiment, the DAP10 polypeptide or variant thereof is endogenous. Alternatively, the DAP10 polypeptide or variant thereof may be exogenous.

The DAP10 polypeptide may be mammalian, for example, murine (such as mouse or rat) or human. In an embodiment, the DAP10 polypeptide is human. Wild-type human DAP10 has the amino acid sequence having UniProt accession no: Q9UBK5 (SEQ ID NO:26). This is a 93aa polypeptide. The first 18aa are considered to be a signal/leader sequence, amino acids 19-48 the extracellular domain, amino acids 49-69 the transmembrane domain, and amino acids 70-93 the cytoplasmic/intracellular domain.

Thus, in an embodiment, the DAP10 polypeptide may comprise or consist of SEQ ID NO:26.

Truncated versions of a DAP10 polypeptide may also be used as a DAP10 polypeptide of the disclosure. For example, a truncated version of DAP10 comprising only amino acids 19-93 of SEQ ID NO:26 (i.e. lacking amino acids 1-18, the signal/leader sequence) may be used as the DAP10 polypeptide of the disclosure. Such a sequence is referred to as SEQ ID NO:27 herein. In an embodiment, the DAP10 polypeptide comprises or consists of SEQ ID NO:27.

Another truncated version of DAP10 used in the invention may comprise amino acids 70-93 of SEQ ID NO:26 (i.e. the intracellular domain), referred to as SEQ ID NO:28 herein. The DAP10 polypeptide may comprise or consist of SEQ ID NO:28.

Other truncated versions may comprise amino acids 19-69 of SEQ ID NO:26, such a sequence comprising merely the extracellular and transmembrane domains of DAP10, and referred to herein as SEQ ID NO:29. A further truncated version of DAP10 used in the invention may comprise amino acids 1-71 of SEQ ID NO:26 (i.e. the signal/leader sequence, extracellular domain, transmembrane domain and 2 amino acids from the cytoplasmic/intracellular domain), referred to as SEQ ID NO:30 herein. A further truncated version of DAP10 used in the invention may comprise amino acids 19-71 of SEQ ID NO:26 (i.e. the extracellular domain, transmembrane domain and 2 amino acids from the cytoplasmic/intracellular domain), referred to as SEQ ID NO:31 herein. A yet further truncated version of DAP10 used in the invention may comprise amino acids 49-93 of SEQ ID NO:26 (i.e. the transmembrane and cytoplasmic/intracellular domains), referred to as SEQ ID NO:32 herein. A yet further truncated version of DAP10 used in the invention may comprise amino acids 49-69 of SEQ ID NO:26 (i.e. the transmembrane domain), referred to as SEQ ID NO:33 herein.

In another embodiment the DAP10 polypeptide or variant thereof is murine, optionally mouse. Wild-type mouse DAP10 has the amino acid sequence having UniProt accession no: Q9QUJ0 (SEQ ID NO:34). This is a 79aa polypeptide. The first 17aa are considered to be a signal/leader sequence, amino acids 18-35 the extracellular domain, amino acids 36-56 the transmembrane domain, and amino acids 57-79 the cytoplasmic/intracellular domain.

Thus, in an embodiment, the DAP10 polypeptide may comprise or consist of SEQ ID NO:34.

Truncated versions of a mouse DAP10 polypeptide may also be used as the DAP10 polypeptide of the disclosure. For example, a truncated version of mouse DAP10 comprising only amino acids 18-79 of SEQ ID NO:34 (i.e. lacking amino acids 1-18, the signal/leader sequence) may be used as the DAP10 polypeptide of the disclosure. Such a sequence is referred to as SEQ ID NO:35 herein. The DAP10 polypeptide may comprise or consist of SEQ ID NO:35.

Another truncated version of a mouse DAP10 polypeptide is SEQ ID NO:36. SEQ ID NO:36 comprises only amino acids 57-79 (intracellular region) of SEQ ID NO:34. Thus, in an embodiment, the DAP10 polypeptide comprises or consists of SEQ ID NO:36.

As the skilled person will appreciate, DAP10 is a homodimer. Thus, in an embodiment, the immunoresponsive cell comprises a DAP10 homodimer comprising two DAP10 polypeptides according to the disclosure. In an embodiment, the immunoresponsive cell comprises a DAP10 heterodimer, each peptide of the DAP10 heterodimer comprising a different DAP10 polypeptide of the disclosure. At least one of the DAP10 polypeptides of the heterodimer may comprise a DAP10 fusion polypeptide as described herein.

Variants of the DAP10 polypeptide are also suitable for use in the invention and are encompassed by the disclosure, as described below. It will be appreciated that any variant of the DAP10 polypeptide will be a functional variant, in that the variant will substantially retain the functional activity of the DAP10 polypeptide on which the variant is based, or even improve the functional activity. In one embodiment, the activity may be measured by assessment of tyrosine phosphorylation of DAP10 and/or recruitment and activation of the p85 subunit of phosphatidylinositol 3-kinase and the downstream anti-apoptotic kinase, AKT.

A variant of the DAP10 polypeptide may have at least 70%, 75%, 80%, 85%, 90%, 95%, 97% or 99% sequence identity to a human DAP10 polypeptide (such as any of SEQ ID NO:26, 27, 28, 29, 30, 31, 32 or 33) or a murine (optionally mouse) DAP10 polypeptide (such as SEQ ID NO:34, 35 or 36). The variant may have at least 90% or at least 95% sequence identity to a human DAP10 polypeptide or a murine, optionally mouse DAP10 polypeptide.

In an embodiment, the variant of the DAP10 polypeptide comprises a peptide comprising one or more point (i.e. 1, 2, 3, 4, 5, 6, 7, 8, 9, 10 or more) mutations that add, delete or substitute any of the amino acids compared to any of SEQ ID NOs 26-36. Optionally, the variant of the DAP10 polypeptide comprises a peptide comprising one or more point (i.e. 1, 2, 3, 4, 5, 6, 7, 8, 9, 10 or more) mutations that add, delete or substitute any of the amino acids compared to any of SEQ ID NOs 26-33. Alternatively, the variant of the DAP10 polypeptide comprises a peptide comprising one or more point (i.e. 1, 2, 3, 4, 5, 6, 7, 8, 9, 10 or more) mutations that add, delete or substitute any of the amino acids compared to any of SEQ ID NOs 34-36.

Hexamer Complex

Wild-type NKG2D has been shown to form a hexamer receptor complex in humans, composed of one NKG2D homodimer assembled with two DAP10 homodimers. DAP12 has also been shown to be capable of associating with wild-type mouse NKG2D to form the hexamer receptor complex. Various hexamer complex formations can thus be expressed in the immunoresponsive cell of the disclosure. In embodiments comprising only the chimeric NKG2D protein (for example, without exogenous DAP10 or DAP12), the chimeric NKG2D protein is capable of associating with endogenous DAP10 and/or DAP12 in the immunoresponsive cell to form a hexamer complex. In other embodiments comprising the chimeric NKG2D protein and exogenous DAP10 and/or DAP12, the chimeric NKG2D protein is capable of associating with the exogenous DAP10 and/or DAP12 to form a hexamer complex. Exemplary hexamer complexes of the invention include those formed from N1, N2, N3 or N5 peptides of the invention, as disclosed herein. Each of the N1, N2, N3 and N5 peptides comprise DAP12 polypeptide regions. Thus, exemplary hexamer complexes comprising the N1, N2, N3 or N5 peptides of the invention may comprise the chimeric NKG2D protein associated with two exogenous DAP12 homodimers (as comprised within each of the N1, N2, N3 and N5 peptides).

It will be appreciated that the DAP12 peptide and the chimeric NKG2D protein of the N1, N2, N3 and N5 peptides may be separated by a cleavable linker. Thus, in an alternative embodiment, the chimeric NKG2D protein of the N1, N2, N3 or N5 peptide may associate with two endogenous DAP10 homodimers to form an exemplary hexamer complex. In another embodiment, the chimeric NKG2D protein of the N1, N2, N3 or N5 peptide may associate with one exogenous DAP12 homodimer and one endogenous DAP10 homodimer to form another exemplary hexamer complex.

The chimeric NKG2D hexamer complex may comprise (i) the chimeric NKG2D protein as described herein, and (ii) at least one DNAX-activating protein 12 (DAP12) polypeptide or a variant thereof or at least one DNAX-activating protein 10 (DAP10) polypeptide or variant thereof. In an embodiment, the chimeric NKG2D hexamer complex comprises (i) the chimeric NKG2D protein as described herein and (ii) at least one DNAX-activating protein 12 (DAP12) polypeptide or a variant thereof and (iii) at least one DNAX-activating protein 10 (DAP10) polypeptide or variant thereof. The chimeric NKG2D hexamer complex may comprise a DAP12 homodimer comprising DAP12 polypeptides and/or a DAP10 homodimer comprising DAP10 polypeptides.

In an embodiment, the chimeric NKG2D hexamer complex is selected from the group consisting of N1, N2, N3, and N5.

The present invention also provides a polypeptide for generating the chimeric NKG2D hexamer complex, wherein the polypeptide comprises (i) the chimeric NKG2D protein, (ii) at least one DNAX-activating protein 12 (DAP12) polypeptide or a variant thereof and/or at least one DNAX-activating protein 10 (DAP10) polypeptide or variant thereof, and (iii) one or more cleavage sites. In an embodiment, the polypeptide comprises a sequence selected from SEQ ID NO: 66-69.

N-Terminal Sequences and C-Terminal Sequences

Various sequences may be attached to the N- or C-terminus of the chimeric NKG2D protein of the disclosure, or to the DAP10 and/or DAP12 polypeptides (or variants thereof) disclosed herein. These may be functional, such as signal peptides, purification tags/sequences, or half-life extension moieties, or may simply comprise spacer sequences. Alternatively, they may comprise a function, such as a T-cell stimulatory function.

In an embodiment, the chimeric NKG2D protein, the DAP10 polypeptide and/or DAP12 polypeptide (or variant(s) thereof) is not attached to a green fluorescent protein (GFP) sequence. In other words, in an embodiment, one or more of the polypeptides/proteins of the invention does not comprise a GFP sequence.

Signal Peptides

Any of the polypeptides described herein may further comprise a signal peptide (otherwise referred to as a leader sequence). In particular, the DAP10 polypeptide or variant thereof and/or the DAP12 polypeptide or variant thereof, may further comprise a signal peptide. The signal peptide may optionally be fused to the N terminus of the polypeptide.

Various peptides are suitable as signal peptides for the polypeptides of the disclosure. One suitable signal peptide is the CD8a signal peptide sequence (amino acids 1-21 of UniProt: P01732 or a shortened derivative comprising amino acids 1-18). This is a commonly used T-cell sequence, and is referred to as SEQ ID NO:37 herein. Thus, in an embodiment, the signal peptide is derived from a CD8a signal peptide. The signal peptide may comprise or consist of SEQ ID NO:37.

In an embodiment, a signal peptide is fused to the N terminus of the DAP10 polypeptide or variant thereof. The signal peptide may comprise SEQ ID NO:38 (aa 1-17 of SEQ ID NO:34).

In one embodiment, a signal peptide is fused to the N terminus of the DAP12 polypeptide or variant thereof. In an embodiment, the signal peptide comprises or consists of SEQ ID NO: 39 (aa 1-21 of SEQ ID NO:21) or SEQ ID NO:40 (aa 1-21 of SEQ ID NO:16).

Variants of the signal peptide are also encompassed by the disclosure. It will be appreciated that a variant of the signal peptide will be a functional variant, in that the variant will substantially retain the functional activity of the signal peptide on which the variant is based, or even improve the functional activity. The variant may have at least 70%, 75%, 80%, 85%, 90%, 95%, 97% or 99% sequence identity to any of SEQ ID NOs:37-40. Optionally, the variant of the signal peptide comprises one or more point (i.e. 1, 2, 3, 4, 5, 6, 7, 8, 9, 10 or more) mutations that add, delete or substitute any of the amino acids compared to any of SEQ ID NOs:37-40.

Purification Tags and Markers

A variety of tags or markers may be attached to the N- or C-terminus of the polypeptides of the disclosure to assist with purification. Any affinity tag may be combined with the polypeptides of the disclosure to assist with purification. Examples of such affinity tags are a His-tag, a FLAG-tag, Arg-tag, T7-tag, Strep-tag, S-tag, aptamer-tag, V5 tag, AviTag™, myc epitope tag or any combination of these tags. In one embodiment the affinity tag is a His-tag (usually comprising 5-10 histidine residues), for example a 6His tag (i.e. HHHHHH) (SEQ ID NO: 41). In another embodiment the affinity tag is a FLAG tag (i.e. DYKDDDDK) (SEQ ID NO: 42). In another embodiment, the affinity tag is an AviTag™ (i.e. GLNDIFEAQKIEWHE) (SEQ ID NO: 43). In another embodiment, the affinity tag is a V5 tag (GKPIPNPLLGLDST) (SEQ ID NO: 44) or (IPNPLLGLD) (SEQ ID NO: 45). In another embodiment, the affinity tag is a myc epitope tag recognised by the 9e10 antibody (EQKLISEEDL) (SEQ ID NO: 46). Various other tags for use in the disclosure are well known in the art.

Combinations of such affinity tags may also be used, either comprising one or more tags at the N-terminus, one or more tags at the C-terminus, or one or more tags at each of the N-terminus and the C-terminus. Examples of such combinations include a His tag (H) combined with an AviTag (A), or a His tag (H) combined with both an AviTag (A) and a FLAG tag (F). The tags may be in either orientation, thus the AviTag/His tag may have the orientation N-AH-C or N-HA-C, while the Avi/His/FLAG tag may have the orientation N-AHF-C, N-FHA-C, etc.

In an embodiment, a DAP10 or DAP12 polypeptide, or variant thereof, comprises a FLAG tag (i.e. DYKDDDDK) (SEQ ID NO:42). In particular, the DAP12 polypeptide or variant thereof may comprise a FLAG tag (SEQ ID NO:42). The FLAG tag may be positioned at or towards the N terminus of the polypeptide, for example a DAP12 polypeptide or variant thereof.

Fusion Polypeptides Comprising DAP10 and/or DAP12

The disclosure provides various fusion polypeptides.

In an embodiment, the DAP10 polypeptide, or variant thereof, is fused to the chimeric NKG2D protein, such that the chimeric NKG2D protein further comprises the DAP10 polypeptide or variant thereof. In one embodiment, fusion is to the N terminus of the chimeric NKG2D protein. Fusion of the DAP10 polypeptide, or variant thereof, may be to the murine NKG2D transmembrane domain or a variant thereof in the chimeric NKG2D protein. Fusion may be direct or may be by a linker.

In another embodiment, the DAP12 polypeptide, or variant thereof, is fused to the chimeric NKG2D protein. Optionally, fusion is to the N terminus of the chimeric NKG2D protein. In an embodiment, a DAP12 intracellular domain, as described herein, is fused to the chimeric NKG2D protein. Fusion of the DAP12 polypeptide, or variant thereof, may be to the murine NKG2D transmembrane domain or a variant thereof in the chimeric NKG2D protein. Optionally, the DAP12 intracellular domain used in such a fusion construct is human. For example, the DAP12 intracellular domain used in such a fusion construct may comprise or consist of SEQ ID NO: 17.

Fusion is optionally using a linker which comprises a cleavage site. In such embodiments, the linker is cleaved to separate the DAP12 polypeptide or variant thereof and the chimeric NKG2D protein.

The disclosure further provides a fusion polypeptide, comprising DAP10 and/or DAP12 polypeptide(s), or variant(s) thereof.

In an embodiment, at least one DAP12 polypeptide or variant thereof is fused to at least one DNAX-activating protein 10 (DAP10) polypeptide or variant thereof. Any of the DAP12 and DAP10 polypeptides and variants thereof disclosed herein are suitable for such a fusion protein. For example, the DAP12 polypeptide or variant thereof may be fused to a human DAP10 extracellular domain peptide sequence (such as SEQ ID NO:78). The DAP12 polypeptide may comprise a human DAP12 transmembrane domain and a human DAP12 intracellular domain (amino acids 41-113 of UniProt accession no: 043914), for example SEQ ID NO:47. Fusion may be direct or by a linker.

The fusion polypeptide may comprise the DAP12 and/or DAP10 polypeptide, or variant(s) thereof, fused to an immune signalling receptor polypeptide comprising an immunoreceptor tyrosine-based activation motif (ITAM). An ITAM is a conserved sequence of four amino acids that is repeated twice in the cytoplasmic tails of non-catalytic tyrosine phosphorylated receptors.

The DAP12 and/or DAP10 polypeptide, or variant(s) thereof, and the ITAM may be directly fused together. Alternatively, they may be joined by a linker. The ITAM may be fused to the N or the C terminus of the DAP12 and/or DAP10 polypeptide or variant(s) thereof. Optionally, the ITAM is fused to the C terminus of the DAP12 and/or the DAP10 polypeptide or variant(s) thereof.

The zeta chain of a T-cell receptor, the eta chain of a T-cell receptor, the delta chain of a T-cell receptor, the gamma chain of a T-cell receptor, or the epsilon chain of a T-cell receptor (i.e. CD3 chains) or the gamma subunit of the FcR1 receptor may comprise the ITAM. In an embodiment, a CD3-zeta chain or gamma subunit of the FcR1 receptor comprises the ITAM.

Various T-cell co-stimulatory activation sequences are known from previous work to engineer CAR-T-cells. These may also be added to fusion polypeptides of the disclosure.

The 4-1BB endodomain (amino acids 214-255 of UniProt accession no: Q07011) may also be used as an N- or C-terminal sequence. The 4-1BB endodomain is referred to as SEQ ID NO:48 herein. The 4-1BB endodomain may act as a co-stimulatory domain.

The CD27 endodomain (amino acids 213-260 of UniProt accession no: P26842) may also be used as an N- or C-terminal sequence. The CD27 endodomain is referred to as SEQ ID NO:49 herein. The CD27 endodomain may act as a co-stimulatory domain.

The human IgG1 hinge (amino acids 218-229 of UniProt accession no: PODOX5) may also be used as an N- or C-terminal sequence. The human IgG1 hinge is referred to as SEQ ID NO:50.

A truncated CD8a hinge (amino acids 138-182 of UniProt accession no: P01732) may also be used as an N- or C-terminal sequence. The truncated CD8a hinge is referred to as SEQ ID NO:51.

Linkers

Suitable linkers can be used to link polypeptides within fusion proteins of the disclosure. The linker may be a peptide linker. Peptide linkers are commonly used in fusion polypeptides and methods for selecting or designing linkers are well-known (see, e.g., Chen X et al., 2013, Adv. Drug Deliv. Rev. 65(10):135701369 and Wriggers W et al., 2005, Biopolymers 80:736-746.).

Peptide linkers generally are categorized as i) flexible linkers, ii) helix forming linkers, and iii) cleavable linkers, and examples of each type are known in the art. In one example, a flexible linker is included in the fusion polypeptides described herein. Flexible linkers may contain a majority of amino acids that are sterically unhindered, such as glycine and alanine. The hydrophilic amino acid Ser is also conventionally used in flexible linkers. Examples of flexible linkers include, without limitation: polyglycines (e.g., (Gly)₄ and (Gly)₅), polyalanines poly(Gly-Ala), and poly(Gly-Ser) (e.g., (Glyₙ-Serₙ)ₙ or (Serₙ-Glyₙ)ₙ, wherein each n is independently an integer equal to or greater than 1).

Peptide linkers can be of a suitable length. The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. For example, a peptide linker can be from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. The peptide linker sequence may be comprised of naturally or non-naturally occurring amino acids, or a mixture of both naturally and non-naturally occurring amino acids.

In an embodiment, the linker comprises the amino acid methionine, optionally at the C-terminus of the linker.

In some aspects, the amino acids glycine and serine comprise the amino acids within the linker sequence. More specifically, the linker sequence may be SGSG (SEQ ID NO:52). In another embodiment, the linker sequence is GSGGG (SEQ ID NO:53). The linker sequence may be GSGG (SEQ ID NO:54).

In other embodiments, a linker may contain glycine (G), serine (S) and proline (P) in a random or repeated patter. In a particular example, n is 1 and the linker is GPPGS (SEQ ID NO:55).

In general, the linker is not immunogenic when administered in a subject, such as a human. Thus, linkers may be chosen such that they have low immunogenicity or are thought to have low immunogenicity.

The linkers described herein are exemplary, and the linker can include other amino acids, such as Glu and Lys, if desired. In certain aspects, peptide linkers may also include cleavable linkers.

The linkers may comprise further domains and/or features, such as a furin cleavage site (RRKR)(SEQ ID NO:56), a P2A ribosomal skip peptide (ATNFSLLKQAGDVEENPGP)(SEQ ID NO:57) and/or a T2A ribosomal skip peptide (EGRGSLLTCGDVEENPGP)(SEQ ID NO: 58). Examples of linkers comprising these domains include SGSG+a P2A ribosomal skip peptide (SGSGATNFSLLKQAGDVEENPGP)(SEQ ID NO:59), SGSG+a T2A ribosomal skip peptide (SGSGEGRGSLLTCGDVEENPGP)(SEQ ID NO:60), and versions also including a furin cleavage site, i.e. furin cleavage site+SGSG+a P2A ribosomal skip peptide (RRKRSGSGATNFSLLKQAGDVEENPGP) (SEQ ID NO:61) and furin cleavage site+SGSG+a T2A ribosomal skip peptide (RRKRSGSGEGRGSLLTCGDVEENPGP) (SEQ ID NO:62). Alternative ribosomal skip peptides that may be used in the invention include F2A (VKQTLNFDLLKLAGDVESNPGP) (SEQ ID NO:63) and E2A (QCTNYALLKLAGDVESNPGP) (SEQ ID NO:64).

The furin cleavage site, P2A ribosomal skip peptide or T2A ribosomal skip peptide may comprise an additional methionine at the C-terminus. An exemplary linker comprising an additional methionine is SEQ ID NO:65, which includes SGSG+a P2A ribosomal skip peptide (SGSGATNFSLLKQAGDVEENPGP)(SEQ ID NO:59)+a methionine (M).

Immunoresponsive Cell

The immunoresponsive cell of the disclosure is a T-cell, natural killer (NK) cell, macrophage, or neutrophil. In an embodiment, immunoresponsive cell is selected from the group consisting of an αβ T-cell, γδ T-cell, a Natural Killer (NK) cell, a Natural Killer T (NKT) cell, a CD4⁺ T-cell, a CD8⁺ T-cell, or any combination thereof.

In an embodiment, the immunoresponsive cell is selected from the group consisting of a CD4⁺ T-cell, a CD8⁺ T-cell, a NK (NK) cell, or any combination thereof. Optionally, the immunoresponsive cell is a CD4+ or a CD8⁺ T-cell.

In one embodiment, the immunoresponsive cell is a T-cell. In another embodiment, the immunoresponsive cell is a αβ T-cell or a γδ T-cell. Optionally, the immunoresponsive cell is a CD8⁺ T-cell.

In an embodiment, the immunoresponsive cell is a CD4⁺ T-cell.

The immunoresponsive cell may be a primary cell. By “primary cell” this will be understood to refer to a cell that has been obtained from a subject—i.e. it is not a cell from a cell line.

The immunoresponsive cell may be a primary T-cell.

Alternatively, the immunoresponsive cell may be from a cell line.

In an embodiment, the immunoresponsive cell is a human cell, optionally a human primary cell. In another embodiment, the immunoresponsive cell is a human primary T-cell.

Exemplary Constructs

The present disclosure provides the following exemplary chimeric NKG2D protein polypeptide constructs in Table 1:

TABLE 1
Exemplary chimeric NKG2D protein and DAP10 or 12 polypeptides

| Name | Sequence |
|---|---|
| CD8α leader-FLAG | MALPVTALLLPLALLLHAARPDYKDDDDKLRPVQAQAQSDCSCSTVSPG |
| tag-DAP12 human | VLAGIVMGDLVLTVLIALAVYFLGRLVPRGRGAAEAATRKQRITETESP |
| extracellular, | YQELQGQRSDVYSDLNTQRPYYKRRKRSGSGATNFSLLKQAGDVEENPG |
| transmembrane and | PMLCARPRRSPAQEDGKVYINMPGRGKISPMFVVRVLAIALAIRFTLNT |
| intracellular | LMWLAIFKETFQPVLFNQEVQIPLTESYCGPCPKNWICYKNNCYQFFDE |
| domains-linker | SKNWYESQASCMSQNASLLKVYSKEDQDLLKLVKSYHWMGLVHIPTNGS |
| comprising furin | WQWEDGSILSPNLLTIIEMQKGDCALYASSFKGYIENCSTPNTYICMQR |
| cleavage site, SGSG | TV |
| sequence, P2A | |
| sequence and | |
| additional | |
| methionine-DAP10 | |
| human intracellular | |
| domain-murine | |
| NKG2D | |
| transmembrane | |
| domain-human | |
| NKG2D extracellular | |
| domain (SEQ ID | |
| NO: 66), may | |
| otherwise be referred | |
| to as N1 peptide | |
| CD8α leader-FLAG | MALPVTALLLPLALLLHAARPDYKDDDDKLRPVQAQAQSDCSCSTVSPG |
| tag-DAP12 human | VLAGIVMGDLVLTVLIALAVYFLGRLVPRGRGAAEAATRKQRITETESP |
| extracellular, | EYQLQGQRSDVYSDLNTQRPYYKRRKRSGSGATNFSLLKQAGDVEENPG |
| transmembrane and | PMVVRVLAIALAIRFTLNTLMWLAIIWSAVFLNSLFNQEVQIPLTESYC |
| intracellular | GPCPKNWICYKNNCYQFFDESKNWYESQASCMSQNASLLKVYSKEDQDL |
| domains-linker | LKLVKSYHWMGLVHIPTNGSWQWEDGSILSPNLLTIIEMQKGDCALYAS |
| comprising furin | SFKGYIENCSTPNTYICMQRTV |
| cleavage site, SGSG | |
| sequence, P2A | |
| sequence and | |
| additional | |
| methionine-murine | |
| NKG2D | |
| transmembrane | |
| domain-human | |
| NKG2D extracellular | |
| domain (SEQ ID | |
| NO: 67), may | |
| otherwise be referred | |
| to as N2 peptide | |
| CD8α leader-FLAG | MALPVTALLLPLALLLHAARPDYKDDDDKLRPVQAQAQSDCSCSTVSPG |
| tag-DAP12 human | VLAGIVMGDLVLTVLIALAVYFLGRLVPRGRGAAEAATRKQRITETESP |
| extracellular, | YQELQGQRSDVYSDLNTQRPYYKRRKRSGSGATNFSLLKQAGDVEENPG |
| transmembrane and | PMKISPMFVVRVLAIALAIRFTLNTLMWLAIFKETFQPVLFNQEVQIPL |
| intracellular | TESYCGPCPKNWICYKNNCYQFFDESKNWYESQASCMSQNASLLKVYSK |
| domains-linker | EDQDLLKLVKSYHWMGLVHIPTNGSWQWEDGSILSPNLLTIIEMQKGDC |
| comprising furin | ALYASSFKGYIENCSTPNTYICMQRTV |
| cleavage site, SGSG | |
| sequence, P2A | |
| sequence and | |
| additional | |
| methionine-murine | |
| NKG2D | |
| transmembrane | |
| domain-human | |
| NKG2D extracellular | |
| domain (SEQ ID | |
| NO: 68), may | |
| otherwise be referred | |
| to as N3 peptide | |
| CD8α leader-FLAG | MALPVTALLLPLALLLHAARPDYKDDDDKLRPVQAQAQSDCSCSTVSPG |
| tag-DAP12 human | VLAGIVMGDLVLTVLIALAVYFLGRLVPRGRGAAEAATRKQRITETESP |
| extracellular, | YQELQGQRSDVYSDLNTQRPYYKRRKRSGSGEGRGSLLTCGDVEENPGP |
| transmembrane and | MIHLGHILFLLLLPVAAAQTTPGERSSLPAFYPGTSGSCSGCGSLSLPL |
| intracellular | LAGLVAADAVASLLIVGAVFLCARPRRSPAQEDGKVYINMPGRGRRKRS |
| domains-linker | GSGATNFSLLKQAGDVEENPGPMKISPMFVVRVLAIALAIRFTLNTLMW |
| comprising furin | LAIFKETFQPVLFNQEVQIPLTESYCGPCPKNWICYKNNCYQFFDESKN |
| cleavage site, SGSG | WYESQASCMSQNASLLKVYSKEDQDLLKLVKSYHWMGLVHIPTNGSWQW |
| sequence and T2A | EDGSILSPNLLTIIEMQKGDCALYASSFKGYIENCSTPNTYICMQRTV |
| sequence-DAP10 | |
| human leader, | |
| extracellular, | |
| transmembrane and | |
| intracellular | |
| domains-linker | |
| comprising furin | |
| cleavage site, SGSG | |
| sequence, P2A | |
| sequence and | |
| additional | |
| methionine- | |
| murine NKG2D | |
| transmembrane | |
| domain-human | |
| NKG2D extracellular | |
| domain (SEQ ID | |
| NO: 69), may | |
| otherwise be referred | |
| to as N5 peptide | |

It will be appreciated that the above constructs, which may otherwise be referred to as N1 to N5 polypeptides, are capable of forming CAR hexamer complexes. The constructs of SEQ ID NOs 66, 68 and 69 each comprise the murine NKG2D transmembrane domain of SEQ ID NO:3. The construct of SEQ ID NO:67 comprises the murine NKG2D transmembrane domain of SEQ ID NO:2.

Each of the exemplary constructs in Table 1 comprise DAP12 polypeptide regions. It will be appreciated that the DAP12 polypeptide of each of the sequences of Table 1 is separated from the next polypeptide (either DAP10 or the chimeric NKG2D protein) using a linker comprising a cleavage site. Thus, this linker is cleaved in the immunoresponsive cell to create a separate DAP12 polypeptide relative to the other peptides. SEQ ID NO:69 comprises an additional linker comprising a cleavage site between the DAP10 polypeptide and the chimeric NKG2D protein, thereby allowing cleavage of the linker to create a separate DAP10 polypeptide. The separate peptides can then self-associate to form a CAR.

The polypeptide sequence comprising the chimeric NKG2D protein sequence may comprise or consist of any one of SEQ ID NOs:66-69.

Variants of the polypeptide comprising the chimeric NKG2D protein sequence are also envisaged. It will be appreciated that a variant will be a functional variant, in that the variant will substantially retain the functional activity of the polypeptide on which the variant is based, or even improve the functional activity. The variant will be capable of forming a CAR complex. In an embodiment, the variant of the polypeptide sequence comprising the chimeric NKG2D protein sequence has at least 70%, 75%, 80%, 85%, 90%, 95%, 97% or 99% sequence identity to any one of SEQ ID NOs:66-69. Optionally, the variant of the polypeptide sequence comprising the chimeric NKG2D protein sequence comprises one or more point (i.e. 1, 2, 3, 4, 5, 6, 7, 8, 9, 10 or more) mutations that add, delete or substitute any of the amino acids compared to any one of SEQ ID NOs:66-69.

Polynucleotides Encoding Chimeric NKG2D Proteins of the Disclosure

Another aspect of the disclosure pertains to isolated polynucleotides that encode the chimeric NKG2D protein of the disclosure. This may be as DNA or RNA.

The isolated polynucleotide(s) may optionally further encode the DAP10 and/or DAP12 polypeptides of the disclosure. A plurality of isolated polynucleotides may encode the chimeric NKG2D protein and DAP10 and/or DAP12 polypeptides of the disclosure. For example, an isolated polynucleotide may encode the chimeric NKG2D protein, with a second isolated polynucleotide encoding the DAP10 and DAP12 proteins. Alternatively, an isolated polynucleotide may encode the chimeric NKG2D protein, with a second isolated polynucleotide encoding DAP10 or DAP12 only. In an embodiment, an isolated polynucleotide encodes the chimeric NKG2D protein, with a second isolated polynucleotide encoding DAP10 and a third isolated polynucleotide encoding DAP12. In another embodiment, an isolated polynucleotide encodes the chimeric NKG2D protein, the DAP12 polypeptide and optionally the DAP10 polypeptide.

Alternatively, the isolated polynucleotide(s) may encode the polypeptide for generating the chimeric NKG2D hexamer complex. This may be as DNA or RNA.

Unless specifically limited herein, the term encompasses nucleic acids containing known analogues of natural nucleotides that have similar properties as the reference nucleic acid and are metabolized in a manner similar to naturally occurring nucleotides. Examples of such analogs include, without limitation, phosphorothioates, phosphoramidates, methyl phosphonates, chiral-methyl phosphorates, 2-O-methyl ribonucleotides, peptide-nucleic acids (PNAs).

Unless otherwise indicated, a particular nucleic acid sequence (which may otherwise be referred to as a polynucleotide) also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with a different base, mixed-base and/or deoxyinosine residues (Batzer et al., 1991, Nucleic Acid Res. 19:5081; Ohtsuka et al., 1985, J. Biol. Chem. 260:2605-2608; and Rossolini et al., 1994, Mol. Cell. Probes 8:91-98).

Substitutions may be used for the practices of codon optimisation and codon wobble, both of which are known to those skilled in the art. Thus, it will be appreciated that the disclosure also encompasses codon-optimised and codon-wobbled polynucleotides. In an embodiment, the isolated polynucleotide is codon-optimised for human expression.

The polynucleotide sequences can be produced by de novo solid-phase DNA synthesis or by PCR mutagenesis of an existing sequence (e.g., sequences as described in the Examples below). Direct chemical synthesis of nucleic acids can be accomplished by methods known in the art, such as the phosphotriester method of Narang et al., 1979, Meth. Enzymol. 68:90; the phosphodiester method of Brown et al., 1979, Meth. Enzymol. 68:109; the diethylphosphoramidite method of Beaucage et al., 1981, Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., 1991, Nucleic Acids Res. 19:967; and Eckert et al., 1991, PCR Methods and Applications 1:17.

Exemplary isolated polynucleotides which encode a chimeric NKG2D protein of the disclosure include any of SEQ ID NOs:70-73.

SEQ ID NO:70 encodes a CD8a leader, a FLAG tag, DAP12 human extracellular, transmembrane and intracellular domains, a linker comprising a furin cleavage site, SGSG sequence, P2A sequence and additional methionine, a DAP10 human intracellular domain, a murine NKG2D transmembrane domain and a human NKG2D extracellular domain. SEQ ID NO:70 encodes the polypeptide of SEQ ID NO:66, which may otherwise be referred to as an N1 peptide.

SEQ ID NO:71 encodes a CD8a leader, FLAG tag, DAP12 human extracellular, transmembrane and intracellular domains, a linker comprising a furin cleavage site, SGSG sequence, P2A sequence and additional methionine, a murine NKG2D transmembrane domain and a human NKG2D extracellular domain. SEQ ID NO:71 encodes the polypeptide of SEQ ID NO:67, which may otherwise be referred to as an N2 peptide.

SEQ ID NO:72 encodes a CD8a leader, a FLAG tag, DAP12 human extracellular, transmembrane and intracellular domains, a linker comprising a furin cleavage site, SGSG sequence, P2A sequence and additional methionine, a murine NKG2D transmembrane domain and a human NKG2D extracellular domain. SEQ ID NO:72 encodes the polypeptide of SEQ ID NO:68, which may otherwise be referred to as N3 peptide.

SEQ ID NO:73 encodes a CD8a leader, a FLAG tag, DAP12 human extracellular, transmembrane and intracellular domains, a linker comprising a furin cleavage site, SGSG sequence and T2A sequence, DAP10 human leader, extracellular, transmembrane and intracellular domains, a linker comprising a furin cleavage site, SGSG sequence, P2A sequence and additional methionine, a murine NKG2D transmembrane domain and a human NKG2D extracellular domain. SEQ ID NO:73 encodes the polypeptide of SEQ ID NO:69, which may otherwise be referred to as an N5 peptide.

The isolated polynucleotide may comprise or consist of any one of SEQ ID NOs:70-73.

Variants of the isolated polynucleotides are also envisaged, and can readily be generated by one skilled in the art based on the peptide sequences provided in this disclosure. In an embodiment, a variant of the isolated polynucleotide has at least 70%, 75%, 80%, 85%, 90%, 95%, 97% or 99% sequence identity to any one of SEQ ID NOs:70-73.

Vectors

The present disclosure also provides one or more vectors comprising one or more isolated polynucleotides of the disclosure.

For example, an isolated polynucleotide encoding a chimeric NKG2D protein and an isolated polynucleotide encoding a DAP12 polypeptide, or a variant thereof, may be on the same vector. Conversely, an isolated polynucleotide encoding a chimeric NKG2D protein and an isolated polynucleotide encoding a DAP12 polypeptide, or a variant thereof, may be on separate vectors (i.e. two or more vectors).

The disclosure provides one or more cloning or expression vectors comprising any of SEQ ID NOs:70-73.

Variants of the cloning or expression vector comprising any of SEQ ID NOs:70-73 are also envisaged. In an embodiment, the variant of the cloning or expression vector comprises an isolated polynucleotide having at least 70%, 75%, 80%, 85%, 90%, 95%, 97% or 99% sequence identity to any one of SEQ ID NOs:70-73.

For expression in host cells, the nucleic acid encoding the chimeric NKG2D protein (and optional further polypeptide(s)) can be present in a suitable vector and after introduction into a suitable host, the sequence can be expressed to produce the encoded chimeric NKG2D protein (and optional further polypeptide(s)) according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). Various expression vectors can be employed to express the polynucleotides encoding the chimeric NKG2D protein (and optional further polypeptide(s)) of the disclosure. Both viral-based and non-viral expression vectors can be used to produce the chimeric NKG2D protein (and optional further polypeptide(s)) of the disclosure in a host cell, such as a mammalian host cell. Non-viral vectors and systems include plasmids, episomal vectors, typically with an expression cassette for expressing a protein or RNA, and human artificial chromosomes (see, e.g., Harrington et al., 1997, Nat Genet. 15:345). For example, non-viral vectors useful for expression of the polynucleotides and polypeptides of the chimeric NKG2D protein (and optional further polypeptide(s)) of the disclosure in mammalian (e.g., human) cells include pThioHis A, B and C, pcDNA3.1/His, pEBVHis A, B and C, (Invitrogen, San Diego, Calif.), MPS V vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, 1995, Annu. Rev. Microbiol. 49:807; and Rosenfeld et al., 1992, Cell 68: 143. In particular, retroviral, lentiviral, adenoviral or adeno-associated viral vectors are commonly used for expression in T-cells. Examples of such vectors include the SFG retroviral expression vector (see Riviere et al., 1995, Proc. Natl. Acad. Sci. (USA) 92:6733-6737). In one embodiment a lentiviral vector is used, these include self-inactivating lentiviral vectors (so-called SIN vectors).

The choice of expression vector depends on the intended host cells in which the vector is to be expressed. Expression vectors for mammalian host cells can include expression control sequences, such as an origin of replication, a promoter, and an enhancer (see, e.g., Queen, et al., 1986, Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPS V promoter, the tetracycline-inducible CMV promoter (such as the human immediate-early CMV promoter), the constitutive CMV promoter, the EF1 alpha promoter, the phosphoglycerate kinase (PGK) promoter and promoter-enhancer combinations known in the art.

Cultures of transformed organisms can be expanded under non-inducing conditions without biasing the population for coding sequences whose expression products are better tolerated by the host cells. In addition to promoters, other regulatory elements may also be required or desired for efficient expression of the antibody of the disclosure or fragments thereof. These elements typically include an ATG initiation codon and adjacent ribosome binding site or other sequences. In addition, the efficiency of expression may be enhanced by the inclusion of enhancers appropriate to the cell system in use (see, e.g., Scharf et al., 1994, Results Probl. Cell Differ. 20:125; and Bittner et al., 1987, Meth. Enzymol., 153:516). For example, the SV40 enhancer or CMV enhancer may be used to increase expression in mammalian host cells.

Host Cell

The disclosure also provides a host cell comprising an isolated polynucleotide(s) of the disclosure. There is also provided a host cell comprising a vector(s) of the disclosure. A host cell comprising a combination of an isolated polynucleotide(s) and vector(s) of the disclosure is also provided.

The isolated polynucleotide(s) or one or more vector(s) may be transfected into a host cell by standard techniques.

The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Alternatively, the isolated polynucleotide(s) or vector(s) may be delivered into the host cell by transduction. For example, a viral vector, as disclosed above, may be used for delivery of the isolated polynucleotide(s) or vector(s).

It is possible to express the chimeric NKG2D protein (and optional further polypeptide(s)) of the disclosure in either prokaryotic or eukaryotic host cells. Representative host cells include many E. coli strains, mammalian cell lines, such as CHO, CHO-K1, and HEK293; insect cells, such as Sf9 cells; and yeast cells, such as S. cerevisiae and P. pastoris. In one embodiment the host cell is an immunoresponsive cell as defined herein. Other types of host cells include induced pluripotent stem cells (iPSCs) and invariant NKT (iNKT) cells. Cell lines which may be used include the NK cell line NK-92.

Mammalian host cells for expressing chimeric NKG2D protein (and optional further polypeptide(s)) of the disclosure include Chinese Hamster Ovary (CHO cells) (including dhfr-CHO cells, described Urlaub and Chasin, 1980, Proc. Natl. Acad. Sci. USA 77:4216-4220 used with a DH FR selectable marker, e.g., as described in R. J. Kaufman and P. A. Sharp, 1982, Mol. Biol. 159:601-621), NSO myeloma cells, COS cells and SP2 cells. In one embodiment the host cells are CHO K1PD cells. In another embodiment the host cells are NSO1 cells. In particular, for use with NSO myeloma cells, another expression system is the GS gene expression system shown in WO 87/04462, WO 89/01036 and EP 338,841. When recombinant expression vectors encoding protein/polypeptide(s) are introduced into mammalian host cells, the chimeric NKG2D protein (and optional further polypeptide(s)) may be produced by culturing the host cells for a period of time sufficient to allow for expression of the chimeric NKG2D protein (and optional further polypeptide(s)) in the host cells.

Kit

Also provided by the present disclosure is a kit comprising the immunoresponsive cell(s), the isolated polynucleotide(s), the vector(s), the pharmaceutical composition, as described below, and/or the host cell(s) of the disclosure.

The kit may further comprise instructions for the use of the immunoresponsive cell(s), isolated polynucleotide(s), one or more vector(s), pharmaceutical composition or host cell(s) of the disclosure. The instructions for use may comprise instructions for use according to the uses and methods disclosed below.

Production Methods

The present disclosure also provides a method of preparing an immunoresponsive cell according to the disclosure. The method comprises introducing the isolated polynucleotide(s) or vector(s) of the disclosure into a T-cell, natural killer (NK) cell, macrophage, or neutrophil.

Introducing the isolated polynucleotide(s) or vector(s) of the disclosure may comprise the steps of (i) transducing an isolated polynucleotide(s) or vector(s) encoding the chimeric NKG2D protein (and optional further polypeptide(s)) of the disclosure into the immunoresponsive cell, and (ii) culturing the immunoresponsive cell such that the chimeric NKG2D protein (and optional further polypeptide(s)) is expressed.

In a further embodiment, the present disclosure provides a method comprising, (i) obtaining T-cells, macrophages, neutrophils and/or NK cells from a subject, (ii) transducing an isolated polynucleotide(s) or vector(s) encoding a chimeric NKG2D protein (and optional further polypeptide(s)) of the disclosure into the T-cells, macrophages, neutrophils and/or NK cells, and (iii) culturing the T-cells, macrophages, neutrophils and/or NK cells such that chimeric NKG2D protein (and optional further polypeptide(s)) is expressed.

Various methods for the culture of immunoresponsive cells are well known in the art. See, for example, Parente-Pereira A C et al. 2014, J. Biol. Methods 1(2):e7, Ghassemi S et al. 2018, Cancer Immunol Res 6(9):1100-1109, and Denman C J et al. 2012, PLoS One 7(1): e30264.

Compositions

The disclosure also provides a pharmaceutical composition(s) comprising the immunoresponsive cell(s), the isolated polynucleotide(s), the vector(s), or the host cell(s) of the disclosure. Such pharmaceutical compositions can comprise a pharmaceutically or physiologically acceptable diluent and/or carrier. The carrier is generally selected to be suitable for the intended mode of administration and can include agents for modifying, maintaining, or preserving, for example, the pH, osmolarity, viscosity, clarity, colour, isotonicity, odour, sterility, stability, rate of dissolution or release, adsorption, or penetration of the composition. Typically, these carriers include aqueous or alcoholic/aqueous solutions, emulsions, or suspensions, including saline and/or buffered media.

Suitable agents for inclusion in the pharmaceutical compositions include, but are not limited to, amino acids (such as glycine, glutamine, asparagine, arginine, or lysine), antimicrobials, antioxidants (such as ascorbic acid, sodium sulphite, or sodium hydrogen-sulphite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), colouring, flavouring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, diluents, excipients and/or pharmaceutical adjuvants.

Parenteral vehicles include sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases, one might include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents, and inert gases, may also be present. The precise formulation will depend on the route of administration. Additional relevant principle, methods and components for pharmaceutical formulations are well known (see, e.g., Allen, Loyd V. Ed, (2012) Remington's Pharmaceutical Sciences, 22^{nd} Edition).

A pharmaceutical composition of the present disclosure can be administered by one or more routes of administration using one or more of a variety of methods known in the art. As will be appreciated by the skilled artisan, the route and/or mode of administration will vary depending upon the desired results. Routes of administration for pharmaceutical compositions of the disclosure include intravenous, intramuscular, intradermal, intraperitoneal, intrapleural, subcutaneous, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrapleural and intra-sternal injection and infusion. In one embodiment, the pharmaceutical composition is administered intratumourally. In an embodiment, administration is intrapleural or intraperitoneal. When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

Alternatively, the pharmaceutical composition described herein can be administered by a nonparenteral route, such as a topical, epidermal, or mucosal route of administration, for example, intranasally, orally, vaginally, rectally, sublingually, or topically.

In certain embodiments, the pharmaceutical composition is for subcutaneous administration. Suitable formulation components and methods for subcutaneous administration of polypeptide therapeutics (e.g., antibodies, fusion polypeptides, and the like) are known in the art, see, for example, US2011/0044977, U.S. Pat. Nos. 8,465,739 and 8,476,239. Typically, the pharmaceutical compositions for subcutaneous administration contain suitable stabilizers (e.g., amino acids, such as methionine, and or saccharides such as sucrose), buffering agents and tonicifying agents.

Typically, in cell therapy, the composition comprising the immunoresponsive cell is administered to the subject by intravenous infusion.

Uses and Methods

The immunoresponsive cell(s), isolated polynucleotide(s), vector(s), host cell(s) or pharmaceutical composition(s) of the disclosure can be administered to a subject and may be used in the treatment of disease, prophylaxis and/or for delaying the onset of disease symptoms.

Thus, the disclosure provides an immunoresponsive cell(s), isolated polynucleotide(s), vector(s), pharmaceutical composition(s) or host cell(s) of the disclosure for use in (i) therapy or (ii) the treatment of cancer. In particular, the disclosure provides the immunoresponsive cell of the disclosure for use in therapy, optionally for use in the treatment of cancer.

The disclosure also provides the use of an immunoresponsive cell(s), polynucleotide(s), pharmaceutical composition(s), one or more vector(s) or host cell(s) of the disclosure in the manufacture of a medicament for the treatment of a pathological disorder.

As used herein, the term “pathological disorder” includes cancer.

The disclosure further provides a method of treating cancer, wherein the method comprises administering to a subject suspected of having or having cancer, an immunoresponsive cell(s), an isolated polynucleotide(s), vector(s), pharmaceutical composition(s) or host cell(s) of the disclosure.

By “suspected of having cancer”, this will be understood to refer to a subject who exhibits one or more symptoms of cancer. Symptoms of cancer may include, but not necessarily be limited to a lump on the body, unexplained bleeding, changes to bowel habits, persistent cough and/or saliva comprising blood, blood in the stool, unexplained anaemia and a change to urination habits.

In embodiments where the immunoresponsive cell is a primary cell, the immunoresponsive cell may be autologous or allogeneic.

“Autologous” will be understood to refer to an immunoresponsive cell obtained from a subject, after which the chimeric NKG2D protein and optional further polypeptides are introduced into the cell. The immunoresponsive cell is then administered back to the same subject (i.e., the immunoresponsive cell is a cell from the subject which has been modified to become an immunoresponsive cell of the disclosure). “Allogeneic” will be understood to refer to an immunoresponsive primary cell obtained from a different subject to the subject to which the immunoresponsive cell is administered.

In an embodiment, the immunoresponsive cell is autologous.

The cancer may include, but not necessarily be limited to, a solid tumour cancer, a soft tissue tumour, a metastatic lesion, and a haematological cancer. For example, the cancer can be liver cancer, lung cancer, breast cancer, prostate cancer, lymphoid cancer, colon cancer, renal cancer, bone cancer, pancreatic cancer, skin cancer, cancer of the head or neck, such as squamous cell carcinoma of the head and neck (SCCHN), cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukaemias including acute myeloid leukaemia, chronic myeloid leukaemia, acute lymphoblastic leukaemia, chronic lymphocytic leukaemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, myelodysplastic syndrome (MDS), chronic myelogenous leukaemia-chronic phase (CMLCP), diffuse large B-cell lymphoma (DLBCL), cutaneous T-cell lymphoma (CTCL), peripheral T-cell lymphoma (PTCL), hepatocellular carcinoma (HCC), gastrointestinal stromal tumours (GIST), non-small cell lung carcinoma (NSCLC), squamous cell carcinoma of the head and neck (SCCHN), environmentally induced cancers including those induced by asbestos, and combinations of said cancers. In embodiments, the cancer is selected from the above group.

In one embodiment, the cancer is selected from the group consisting of cancer of the head and/or neck, ovarian cancer, malignant mesothelioma, breast cancer, pancreatic cancer, colorectal cancer, lung cancer, gastric cancer, bladder cancer, prostate cancer, oesophageal cancer, endometrial cancer, hepatobiliary cancer, duodenal carcinoma, thyroid carcinoma, cancer of the central nervous system or renal cell carcinoma.

In another embodiment, the cancer is selected from ovarian cancer, breast cancer, optionally triple-negative breast cancer, pancreatic cancer, malignant mesothelioma, and combinations of said cancers.

In one embodiment the cancer is ovarian cancer, breast cancer or combinations thereof.

In one embodiment, the subject has been pre-treated with a chemotherapeutic agent.

In one embodiment, the administration of immunoresponsive cells to the subject results in a decrease in tumour size of 10%, 20%, 30%, 40%, 50%, 60%, 70%, 80%, 90%, or even 100%, when compared to an untreated tumour.

The amount of immunoresponsive cells administered to the subject should take into account the route of administration, the cancer being treated, the weight of the subject and/or the age of the subject. In general, about 1×10⁶ to about 1×10¹¹ cells are administered to the subject. In one embodiment, about 1×10⁷ to about 1×10¹⁰ cells, or about 1×10⁸ to about 1×10⁹ cells are administered to the subject.

The disclosure also provides a method for directing a T-cell-mediated immune response to a target cell in a subject in need thereof. The method comprises administering to the subject the immunoresponsive cell(s), the polynucleotide(s), the one or more vector(s), the pharmaceutical composition(s), or the host cell(s) of the disclosure. Typically, the method comprises administering to the subject a plurality of immunoresponsive cells of the disclosure.

### GENERAL

Throughout the description and claims of this specification, the words “comprise” and “contain” and variations of the words, for example “comprising” and “comprises”, mean “including but not limited to”, and do not exclude other components, integers or steps.

Moreover, the singular encompasses the plural unless the context otherwise requires: in particular, where the indefinite article is used, the specification is to be understood as contemplating plurality as well as singularity, unless the context requires otherwise.

By “murine”, this will be understood to refer to a rodent of the subfamily Murinae. The term “murine” comprises rat and mouse.

Sequence identity can be determined by standard methods that are commonly used to compare the similarity in position of the amino acids of two polypeptides. Using a computer program such as BLAST, FASTA or Clustal Omega, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. Sequence identity is typically measured along the full length of the reference sequence.

It will be understood that a substitution of an amino acid may be conservative or non-conservative. By “conservatively substitute”, this will be understood to refer to a replacement of any amino acid with another amino acid in the same class with broadly similar properties. Non-conservative substitutions are where amino acids are replaced with amino acids of a different type or class.

Amino acid classes are defined as follows:

Class Amino Acid Examples

Nonpolar: A, V, L, I, P, M, F, W

Uncharged polar: G, S, T, C, Y, N, Q

Acidic: D, E

Basic: K, R, H.

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions may also be possible provided that these do not interrupt the function of the polypeptide as described above.

As used herein, the term “variant” in the context of a peptide sequence encompasses a peptide sequence which is a naturally occurring polymorphic form of the basic sequence as well as synthetic variants, in which one or more amino acids within the chain are inserted, removed or replaced. However, the variant produces a biological effect which is similar to that of the basic sequence. For example, a variant of the extracellular domain of human NKG2D will act in a manner similar to that of the extracellular domain of human NKG2D.

The variant may otherwise be referred to as a functional variant, in that while one or more of the amino acids within the chain are inserted, removed or replaced, relative to the basic sequence, the variant substantially retains the functional activity of the basic sequence. “Substantially retains” will be understood to refer to a functional activity of at least 80%, at least 85%, at least 90%, at least 95%, at least 97%, at least 98%, at least 99% or at least 100% of the basic sequence. A variant of the present invention may have a functional activity equivalent or improved to the basic sequence.

It will be appreciated that the term “truncated version” in the context of a peptide sequence comprises a shortened version of the basic peptide sequence which produces a biological effect which is equivalent to or improved relative to the basic sequence.

In the context of polynucleotides, it will be appreciated that any “fragment” or “variant” polynucleotide retains the ability to encode a protein similar to or identical to that encoded by the basic polynucleotide.

The term “about” in relation to a numerical value x means, for example, x±5%.

Features of each aspect of the disclosure may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### EXAMPLES

Methods

Retroviral Production by Transient Transfection of HEK 293T Cells

To transiently generate retrovirus, 1.65×10⁶ HEK293T were seeded into a 10 cm² tissue culture dish in 10 mL IMDM containing 10% FBS+2 mM L-glutamine. After overnight incubation at 37° C. and 5% CO₂, the following transfection mixes were generated (volumes given are for a single plate);

Mix A=470 μL serum free IMDM+30 μL Genejuice transfection reagent.

Mix B=4.6875 μg PeqPam-3 plasmid, 3.125 μg RDF plasmid, 4.6875 μg retroviral genome plasmid.

Once generated, Mix A was incubated at room temperature for 5 minutes. Mix B was subsequently added to Mix A, gently mixed and incubated for 15 minutes at room temperature. The complete mixture was added dropwise to the cells, which were subsequently incubated at 37° C. and 5% CO₂ for 48 hours. After 48 hours, the supernatant containing the retrovirus was removed and placed into a pre-chilled 50 mL falcon tube, which was stored in the fridge overnight. The HEK293T cells were fed with 10 mL fresh IMDM and returned to the incubator. After an additional 24 hours, the media was harvested from the HEK293T cells and combined with the supernatant harvested 24 hour previously. The supernatant was aliquoted into 1.5 mL vials and snap frozen using an ethanol bath, before being stored at −80° C. until required.

Activation and Transduction of Peripheral Blood Mononuclear Cells

Human peripheral blood mononuclear cells (PBMCs) were isolated from healthy volunteers using standard Ficoll Paque-mediated density centrifugation. Once re-suspended at a concentration of 3×10⁶ cells/mL in RPMI+5% normal human AB serum, the T-cells were activated using paramagnetic beads coated with anti-human CD3 and anti-human CD28 antibodies (1:2 cell:bead ratio), or phytohaemagglutinin (PHA) at a concentration of 5 ug/mL. Forty-eight hours after activation, 1×10⁶ T-cells were plated onto retronectin-coated non-tissue culture treated plates and mixed with 3 mL viral supernatant harvested from transiently transfected HEK 293T cells. T-cells were fed with 100 IU/mL IL-2 in RPMI1640 media+5% normal human AB serum, with fresh media and IL-2 (100 IU/mL) provided thrice weekly.

Assessment of Gene Transfer Efficiency Using Flow Cytometry

To determine expression of the N1-N5 CARs, T-cells were stained with fluorescein isothiocyanate (FITC)-conjugated anti-human CD4, phycoerythrin (PE)-conjugated anti-human NKG2D and allophycocyanin (APC)-conjugated anti-human CD8a antibodies on ice for 30 minutes. After washing in 2 mL ice-cold PBS, the cells were re-suspended in 0.5 mL ice-cold PBS and assessed by flow cytometry. Due to endogenous expression of NKG2D in CD8⁺ T-cells, gene transfer efficiency was calculated within the CD4⁺ T-cells and compared to that seen in untransduced (UT) T-cells.

Quantification of tumour cell viability using MTT assay (cytotoxicity assay) Tumour cells were co-cultured with CAR T-cells at log 2 effector:target ratios ranging from 1:1 to 1:64. After 72 hours, the T-cells were removed and 100 μL MTT solution (500 μg/mL) added per well. The plates were incubated at 37° C. and 5% CO₂ for approximately 1 hour. Following removal of the MTT solution, the resulting formazan crystals were solubilised in DMSO (100 μL/well) and the absorbance measured at 560 nm. Tumour cell viability was calculated as follows: (Absorbance of monolayer with T-cells/absorbance of monolayer without T-cells)*100.

To assess the ability of the CAR T-cells to undergo multiple rounds of target cell lysis (‘re-stimulation experiments’), the CAR T-cells were co-cultured with tumour cells at a 1:1 effector:target ratio. After 72 hours, the T-cells were gently removed and each well gently washed with 1 mL PBS. Following removal of the PBS, 0.5 mL MTT solution was added per well and the plates incubated at 37° C. and 5% CO₂ for approximately 1 hour. Following removal of the MTT solution, the resulting formazan crystals were solubilised in DMSO (0.5 mL/well) and the absorbance measured at 560 nm. Tumour cell viability was calculated as follows: (Absorbance of monolayer with T-cells/absorbance of monolayer without T-cells)*100. The T-cells that had been removed from the monolayer were spinoculated at 400×g for 5 minutes and the supernatant removed. The pellet was re-suspended in 3.2 mL R5 media and 1 mL added to each well of fresh tumour monolayer in triplicate. This assay was repeated twice weekly until the T-cells failed to mediate greater than 40% target cell lysis. To investigate T-cell proliferation in response to target cell recognition, T-cell number was assessed by trypan blue exclusion of a small aliquot of the remaining 200 μL. Fold-expansion was calculated as follows (highest total T-cell number achieved during re-stimulation/initial T-cell number seeded).

Assessment of Exhaustion/Activation Marker Expression by T-Cells Pre- and Post-Stimulation

The expression of the exhaustion/activation markers Programmed Death-1 (PD-1), Lymphocyte Activation Gene-3 (LAG-3) and T-cell immunoglobulin and mucin-domain containing-3 (TIM-3) were assessed on T-cells prior to (“T-cells alone”) and following one (“Post-stim 1”) or three stimulations (“Post-stim 3”) on tumour cells by flow cytometry. The T-cells were stained with Phycoerythrin-conjugated anti-PD-1, AlexaFluor647-conjugated anti-LAG-3 and Phycoerythrin-cyanine7-conjugated anti-TIM-3. To differentiate between T-cell and any residual tumour cells, samples were also stained with fluorescein isothiocyanate (FITC)-conjugated CD4 and FITC-conjugated CD8 antibodies.

Assessment of Cytokine Secretion by CAR T-Cells Using Enzyme-Linked Immunosorbent Assay (ELISA).

As an additional marker of T-cell activation, the secretion of IFN-γ by CAR T-cells during co-culture with tumour cells was assessed by ELISA. To achieve this, a 200 μL aliquot was removed from each co-culture 24 and 72 hours after addition of the T-cells. The presence of IFN-γ was subsequently assessed using the IFN-γ Duoset ELISA kit from Bio-Techne as per the manufacturer's instructions. Prior to use in the assay, all supernatants were diluted 1:10 in reagent diluent buffer (as detailed by the manufacturer) to ensure they fell within the detection limits of the assay. The percentage increase in IFN-γ cytokine concentration was calculated per donor as follows; (average cytokine concentration per construct/cytokine concentration from NKG2D T-cells)*100

Assessment of CAR Efficacy In Vivo

To test the efficacy of the N1-N5 CAR T-cells against a model of pancreatic cancer in vivo, NOD-SCID-γc^{-/-} (NSG) mice were inoculated intraperitoneally (i.p.) with 1×10⁵ firefly luciferase (ffLUC)-tagged BxPC3 cells. Twelve days after tumour inoculation, mice were treated i.p. with either PBS, or 1×10⁷ N1, N2, N3, N4, N5 or N1012 CAR⁺ T-cells. Tumour growth was measured weekly by bioluminescence imaging, with mice weighed thrice weekly. Mice that were tumour-free at Day 41 (29 days post-T-cell infusion) were re-challenged i.p. with an additional 1×10⁵ ffLUC-tagged BxPC3 cells. Mice were imaged on Day 42 to confirm tumour presence and weekly thereafter until conclusion of the experiment.

In an additional model of pancreatic cancer, SCID-Beige mice were inoculated i.p. with 1×10⁶ ffluc-tagged BxPC3 cells. Seven days post tumour inoculation, mice were treated i.p. with 1×10⁷ N2, N3, N4, N5 or N1012 CAR⁺ T-cells, UT T-cells, or PBS. Tumour growth was monitored weekly by bioluminescence imaging and mice weighed weekly. Mice that remained tumour free at Day 63 were re-challenged i.p. with 1×10⁶ ffLUC-tagged BxPC3 cells. Tumour growth was investigated weekly by bioluminescence imaging.

As a model of ovarian cancer, SCID Beige mice were inoculated i.p. with 1×10⁶ ffLUC-tagged SKOV3 cells. Twenty-one days after tumour inoculation, the mice were treated i.p. with 5×10⁶ N5 or N1012 T-cells, or with PBS. Tumour growth was monitored by weekly bioluminescence imaging and the mice were weighed weekly as a marker of toxicity.

As a model of malignant mesothelioma, NSG mice were inoculated i.p. with 1×10⁶ ffLUC-tagged H226 cells. Eight days after tumour inoculation, mice were treated with either PBS, or 4×10⁶ T-cells expressing either N5, N1012 T-cells. As a control, one group of mice were treated with 4×10⁶ T-cells expressing NKG2D alone. Tumour growth was monitored weekly by bioluminescence imaging and mice were weighed weekly as a marker of toxicity. Mice that were tumour-free 91 days after tumour inoculation were re-challenged i.p. with an additional 1×10⁶ffLUC-tagged H226 cells. Tumour take was confirmed by bioluminescence imaging 24 hours after re-challenge, with tumour growth subsequently monitored weekly by bioluminescence imaging.

To establish a mesothelioma patient-derived xenograft model, a primary patient mesothelioma tumour was cut into chunks of approximately 4.2 mm³ and implanted subcutaneously into the flanks of 5-week-old female NSG mice using a 15 G trocar needle. Unused tumour tissue was formalin fixed and paraffin embedded for H&E staining, or frozen down for future reference. Tumours were harvested from the mice upon reaching 523 mm³, cut into chunks of 4.2 mm³ and re-implanted subcutaneously into a fresh cohort of female NSG mice. Again, unused tissue was stored appropriately for future use. Upon reaching 523 mm³, the tumours were harvested, cut into chunks of 4.2 mm³ and implanted subcutaneously into a third cohort of NSG mice. Mice were treated intravenously with either 4×10⁶ or 1×10⁷ CAR T-cells when the tumours were between 4.2 mm³ and 189 mm³. Tumour growth was measured weekly by caliper measurement.

### Example 1: Engineering and Expression of NKG2D CARS in T-Cells

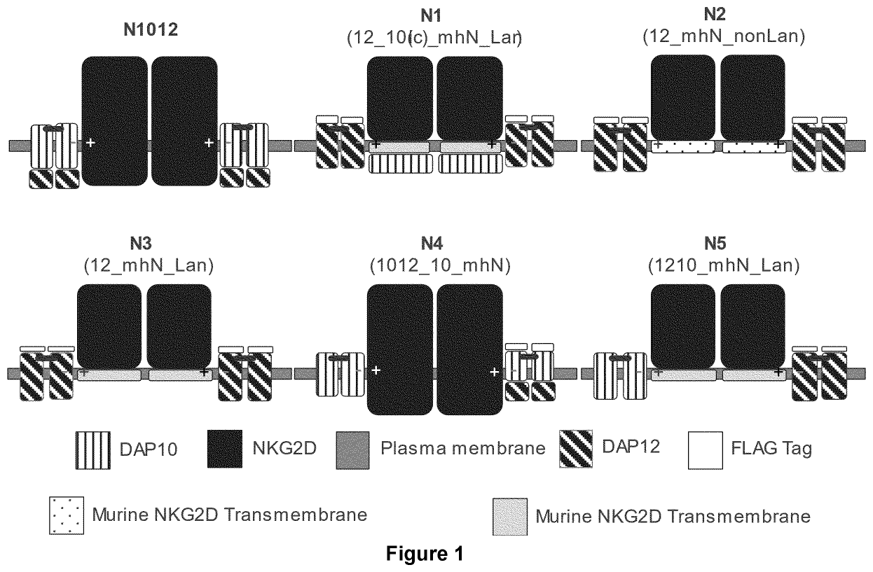

FIG. 1 is a diagram showing features of CARs used in the experiments described herein. The cell membrane is shown as horizontal grey lines, with the extracellular domains depicted above the membrane, transmembrane domains spanning the cell membrane and intracellular domains shown below the membrane. N1012 and N4 comprise CARs which are provided for comparative purposes. N1012 comprises a complex comprising a human NKG2D protein and fusion DAP10/12 homodimers. The human NKG2D protein has a human extracellular, transmembrane, and intracellular NKG2D domain. Each monomer of the fusion DAP10/12 homodimer comprises a human DAP10 extracellular, transmembrane and intracellular domain and a human DAP12 intracellular domain. N1012 consists of the peptide sequence SEQ ID NO:74, and is encoded by the nucleotide sequence SEQ ID NO:75. N4 is a complex which differentiates from N1012 in that it does not contain a human DAP10 intracellular domain. This means that the human DAP12 intracellular domain of N4 is directly at the cell membrane. Thus, N4 comprises a human NKG2D protein having an extracellular, transmembrane, and intracellular domain. N4 also comprises a fusion human DAP10/DAP12 homodimer, each monomer of the fusion DAP10 homodimer comprising a human DAP10 extracellular and transmembrane domain fused to a human intracellular DAP12 domain. Each monomer of the fusion DAP10/DAP12 protein also comprises a FLAG tag at the N-terminus. In addition, N4 comprises exogenous human DAP10 protein. This protein comprises a human signal peptide, extracellular, transmembrane and intracellular domain (SEQ ID NO:26). N4 consists of the peptide sequence SEQ ID NO:76, and is encoded by the nucleotide sequence SEQ ID NO:77.

N1 is an exemplary CAR complex comprising a chimeric NKG2D protein and DAP12 homodimers according to the present invention. The chimeric NKG2D protein comprises a human extracellular NKG2D domain, fused to a murine NKG2D transmembrane domain. The murine NKG2D transmembrane domain is fused to a human DAP10 intracellular domain. Each monomer of the DAP12 homodimer comprises a human extracellular, transmembrane, and intracellular DAP12 domain. The N terminus of each DAP12 monomer comprises a FLAG tag. The N1 CAR (prior to post-translational processing) comprises peptide sequence SEQ ID NO:66, which is encoded by polynucleotide sequence SEQ ID NO:70.

N2 is another exemplary CAR complex comprising a chimeric NKG2D protein and DAP12 homodimers according to the present invention. N2 comprises a human extracellular NKG2D domain, fused to a murine NKG2D transmembrane domain. Each monomer of the DAP12 homodimer comprises a human extracellular, transmembrane, and intracellular DAP12 domain. The N terminus of each DAP12 monomer comprises a FLAG tag. The N2 CAR (prior to post-translational processing) comprises peptide sequence SEQ ID NO:67, which is encoded by polynucleotide sequence SEQ ID NO:71.

Another exemplary CAR complex comprising a chimeric NKG2D protein and DAP12 homodimers according to the present invention is N3. The chimeric NKG2D protein comprises a human extracellular NKG2D domain, fused to a murine NKG2D transmembrane domain. Each monomer of the homodimer comprises a human extracellular, transmembrane, and intracellular DAP12 domain. The N terminus of each DAP12 monomer comprises a FLAG tag. N3 is thus identical to N1, except that it does not comprise a DAP10 intracellular domain. The N3 CAR (prior to translational processing) comprises peptide sequence SEQ ID NO:68, which is encoded by polynucleotide sequence SEQ ID NO:72.

N5 represents another exemplary CAR complex of the invention, which comprises a chimeric NKG2D protein, a DAP10 homodimer and a DAP12 homodimer. The chimeric NKG2D protein comprises a human extracellular NKG2D domain, fused to a murine NKG2D transmembrane domain. Similarly to N3, N5 does not comprise a DAP10 intracellular domain. N5 also comprises a DAP10 homodimer, each monomer of the homodimer comprising a human DAP10 intracellular, transmembrane, and extracellular domain and a human DAP12 homodimer, each monomer of the homodimer comprising a human extracellular, transmembrane, and intracellular DAP12 domain. Each monomer of the DAP12 protein comprises a FLAG tag at its N-terminus. The N5 CAR comprises (prior to post-translational processing) peptide sequence SEQ ID NO:69, which is encoded by polynucleotide sequence SEQ ID NO:73.

Where a CAR complex comprises a DAP10 and/or DAP12 homodimer, it will be appreciated that multiple copies of the peptide sequence (i.e. SEQ ID NO:66-69) are present in the cell. In this way multiple monomers, and thereby homodimers, can be present in the CAR.

In some experiments, CAR complexes of the invention were compared to CYAD-01, CYAD-01_10 or NKG2D T-cells. CYAD-01 is an NKG2D-targeted CAR T-cell which is currently undergoing clinical development by Celyad Oncology and consists of a fusion of NKG2D to the intracellular domain of CD3 (Zhang et al, 2005, Blood 106:1544-1551). In the CYAD-01_10 CAR, additional DAP10 has been stoichiometrically co-expressed with CYAD-01 using a ribosomal skip peptide. NKG2D T-cells are T-cells in which NKG2D was over-expressed alone.

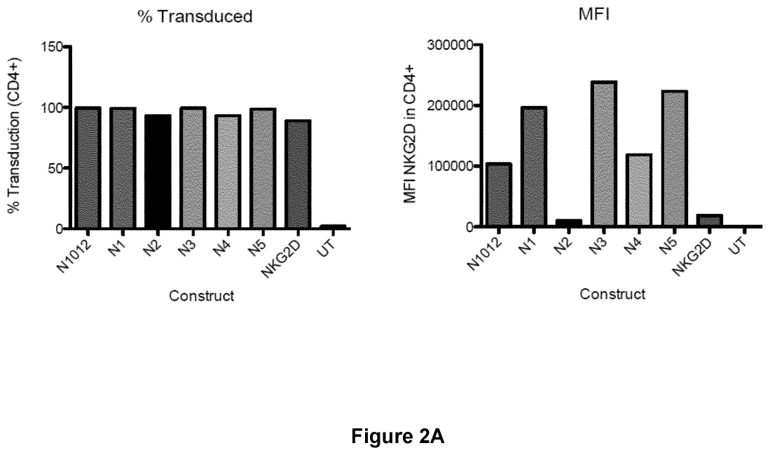

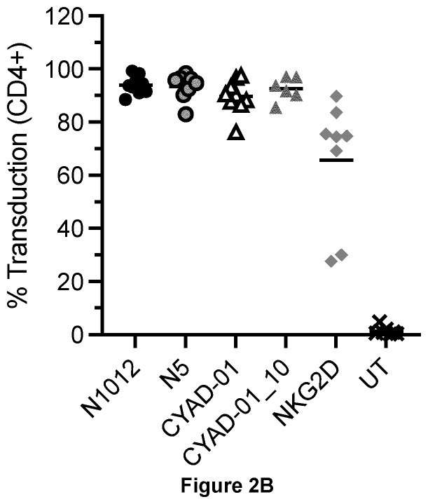

Surface expression of NKG2D was assessed by flow cytometry using a PE-conjugated anti-human NKG2D antibody (FIG. 2A). T-cells were also co-stained with fluorescein isothiocyanate (FITC)-conjugated anti-human CD4 and allophycocyanin (APC)-conjugated anti-human CD8⁺ antibodies. The percentage expression (FIG. 2A, left graph) and median fluorescence intensity (MFI, FIG. 2A, right graph) of NKG2D was compared against untransduced (UT) T-cells. Due to endogenous expression of NKG2D in CD8⁺ T-cells, data are gated on CD4⁺ T-cells. As shown, greater than 95% transduction was achieved with all constructs compared to UT T-cells. Furthermore, aside from N2, all constructs were expressed at very high levels at the surface of primary human T-cells, in contrast to either UT T-cells, or T-cells expressing NKG2D alone. This high level of transduction was reproducible across multiple donors (FIG. 2B). The lower surface expression seen with the N2 construct may reflect sub-optimal interaction of DAP10 and/or DAP12 with the shorter murine NKG2D transmembrane domain present exclusively in this construct.

### Example 2: In Vitro Activity of NKG2D-CAR Expressing T-Cells

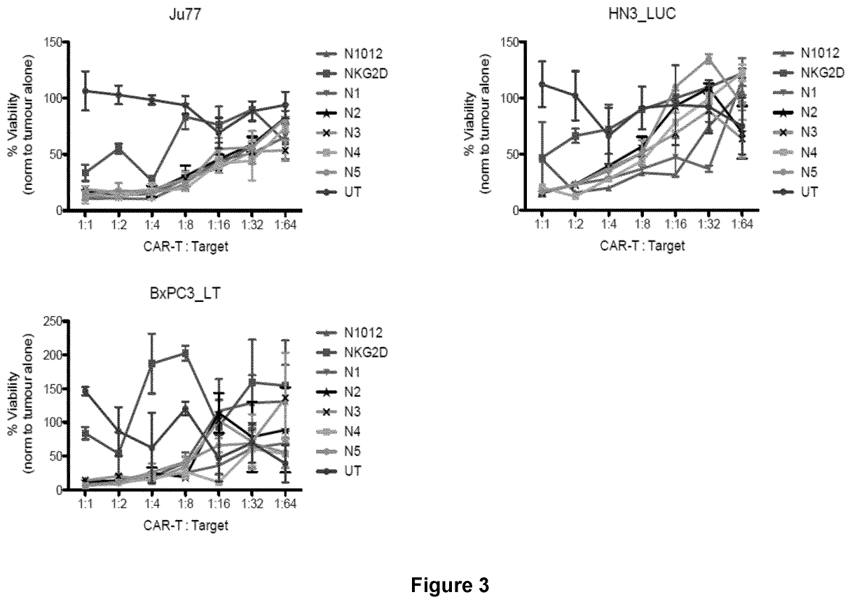

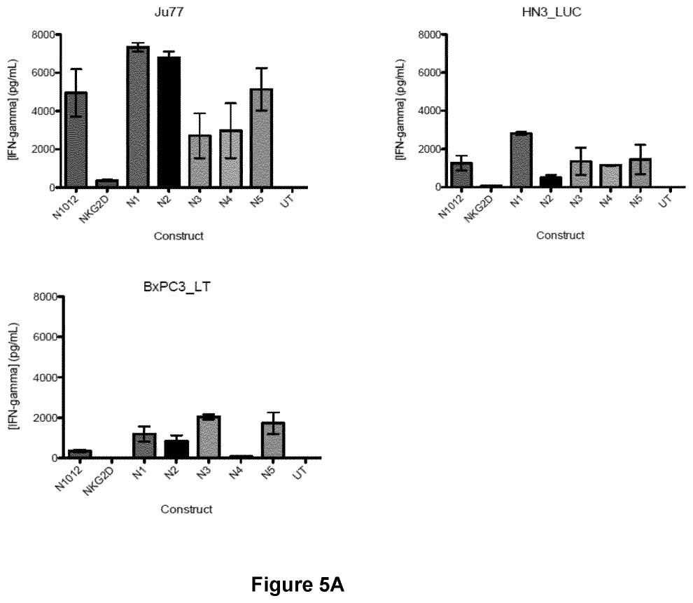

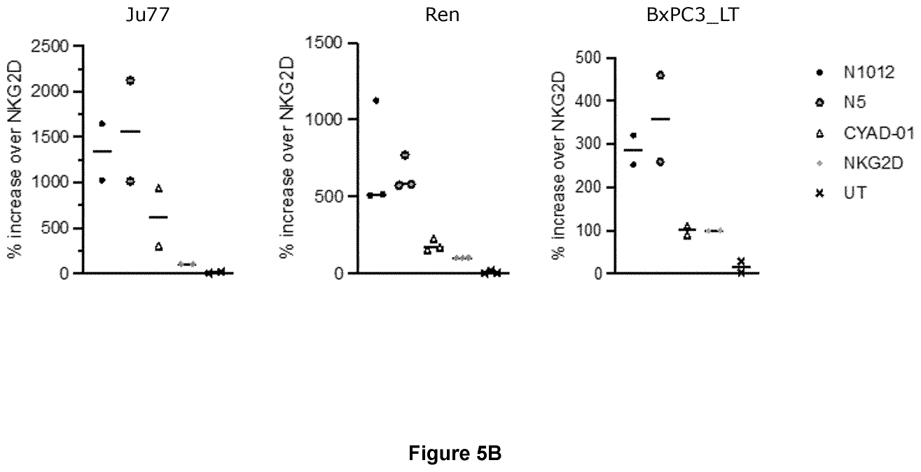

To confirm function of the NKG2D-targeted CARs, the T-cells were co-cultured with either pancreatic cancer (BxPC3_LT), head and neck cancer (HN3_LUC), or malignant mesothelioma (Ju77) cells at effector:target ratios ranging from 1:1-1:64 for 72 hours. Whereas a minimal reduction in tumour viability was observed when target cells were co-cultured with either UT T-cells, or those expressing NKG2D, N1-N5 CAR T-cells demonstrated potent lysis of all tumour cell lines, even at low effector:target ratios (FIG. 3). Analysis of co-culture supernatants by ELISA of the 1:1 effector:target ratio demonstrated substantial secretion of Interferon-γ (IFN-γ) by N1-N5 T-cells, but not by either UT T-cells or those expressing the control NKG2D construct (FIG. 5A). The percentage increase in IFN-γ cytokine expression over NKG2D CAR T-cells is shown in FIG. 5B. These data demonstrate the ability of N1-N5 CAR T-cells to recognise and lyse a broad variety of tumour types.

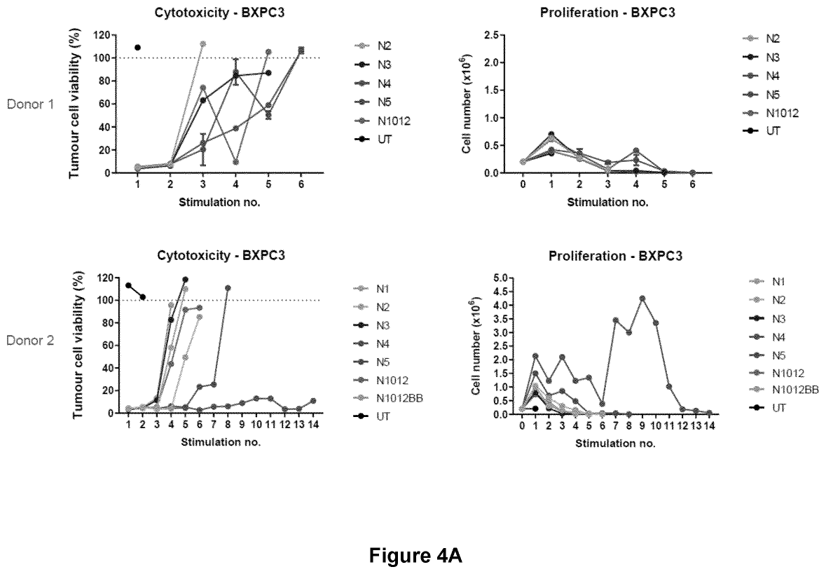

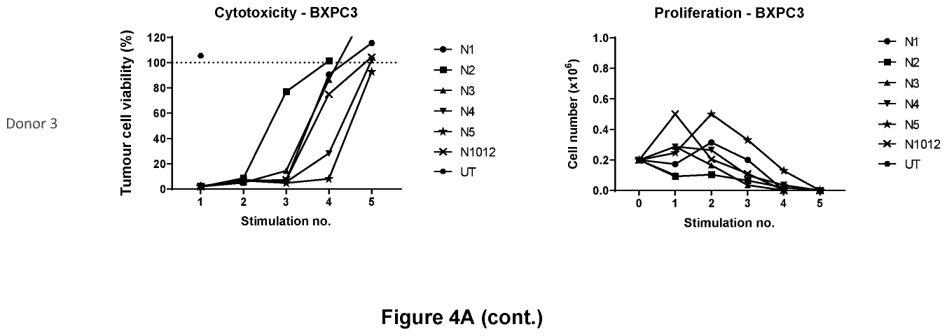

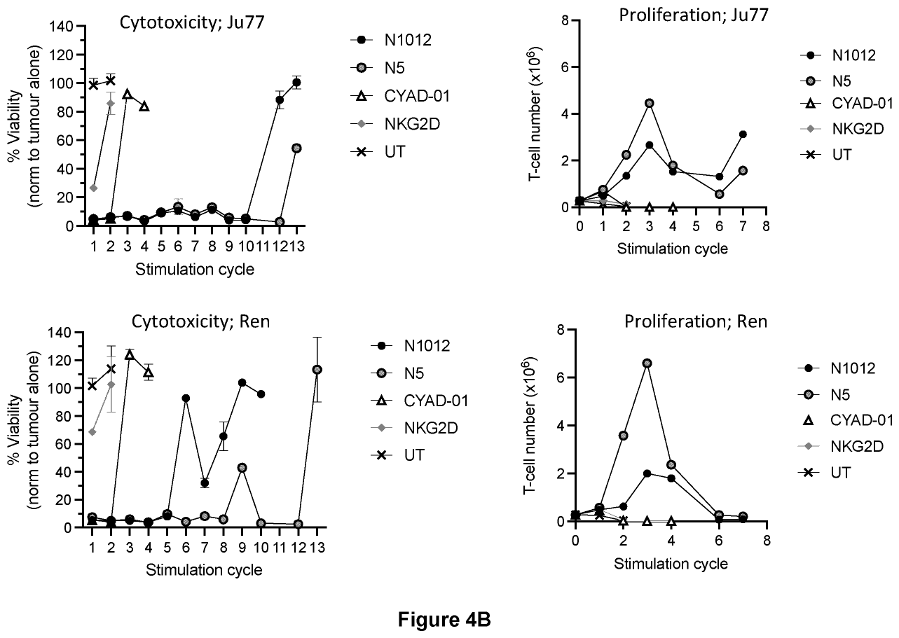

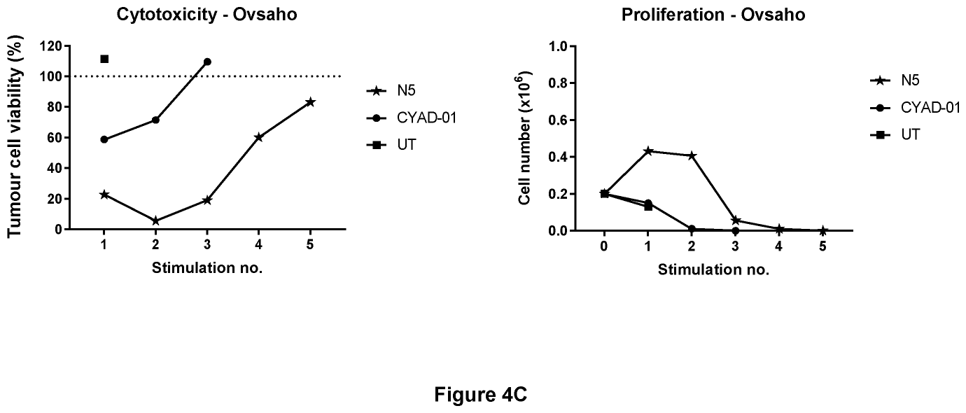

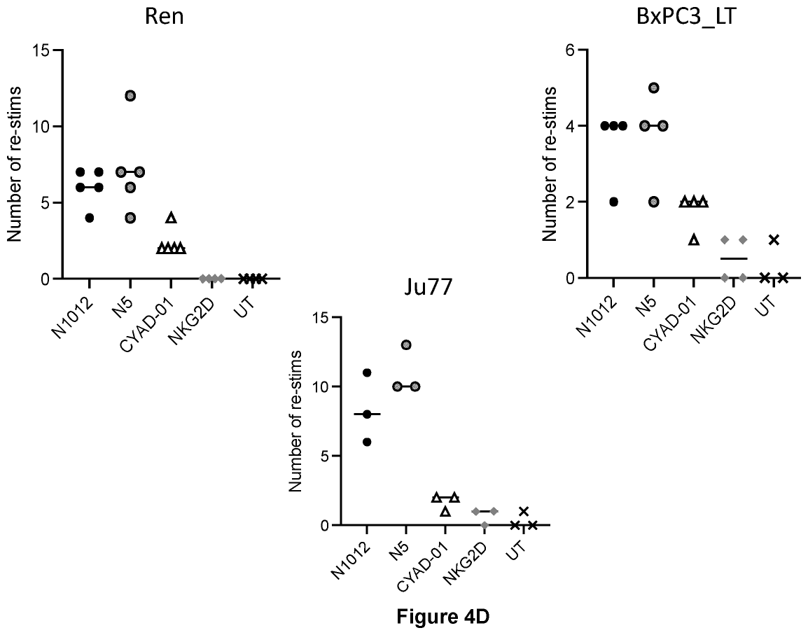

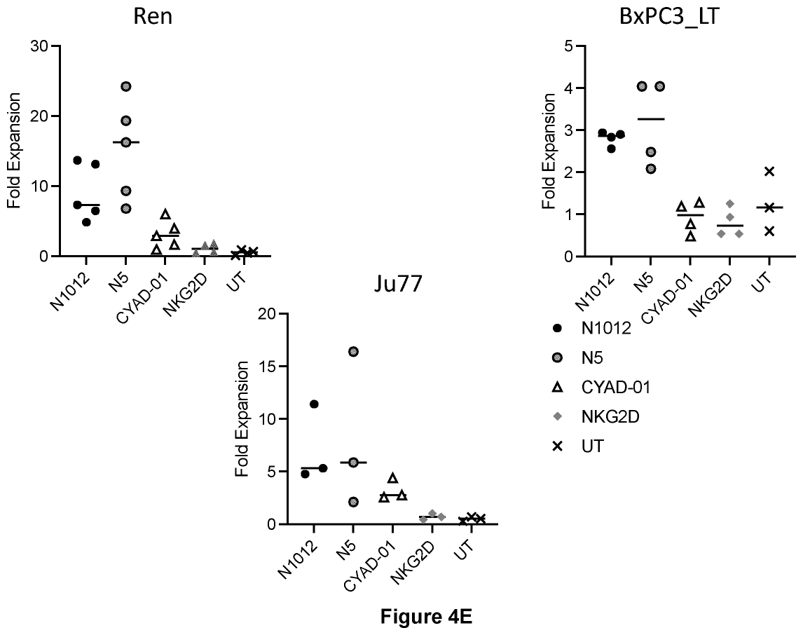

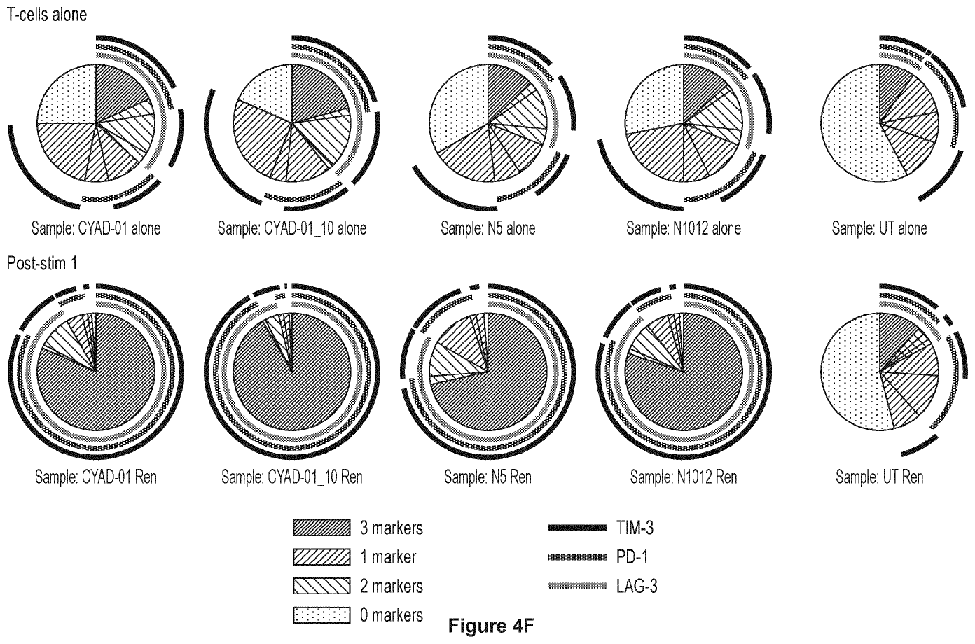

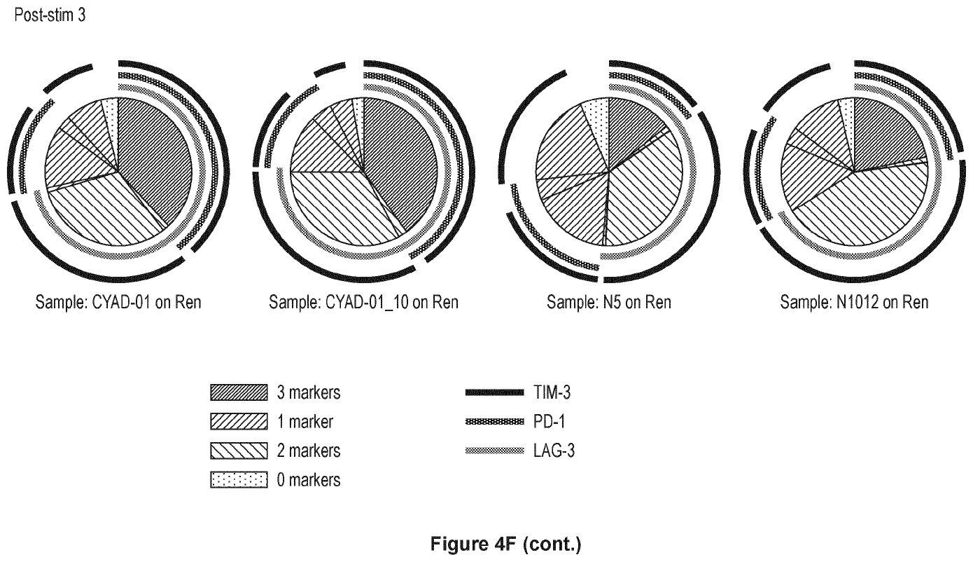

To assess the ability of the N1-N5 CAR T-cells to mediate serial tumour lysis, they were repeatedly stimulated upon ffLUC-tagged BxPC3 cells, Ju77, Ren or OVSAHO tumour cells. The N5 CAR T-cell was also compared to CYAD-01 CAR T-cells. Whereas UT T-cells mediated no discernible reduction in tumour viability, N1-N5 CAR T-cells demonstrated potent cytotoxicity against all tumour cell lines through multiple rounds of stimulation (FIG. 4A to 4C). An increased number of restimulation cycles was observed with N5 cells (FIG. 4D). Target cell destruction was also associated with substantial proliferation of N1-N5 T-cells (FIGS. 4A to 4C and 4E).

The expression of the T-cell exhaustion markers, PD-1, LAG-3 and TIM-3 was assessed by flow cytometry 72 hours after the initial co-culture of CAR T-cells with mesothelioma tumour cells. This was then repeated after three rounds of stimulation. All CAR T-cells had a higher baseline expression of all three markers, when compared to untransduced (UT) T-cells (“T-cell alone”, FIG. 4F). Upon a single round of stimulation (“post-stimi”), the majority of the CAR T-cells expressed all three markers simultaneously. No major change in expression of these molecules was observed in the UT T-cells, thus confirming the requirement of CAR-mediated activation for the change in phenotype. Following the third round of stimulation (FIG. 4F, “post-stim 3”), a substantial proportion of CYAD-01 and CYAD-01_10 T-cells continued to express all three molecules. In contrast, very few N5 or N1012 T-cells simultaneously expressed PD-1, LAG3 and TIM-3, with a substantial reduction in the number of T-cells expressing PD-1.

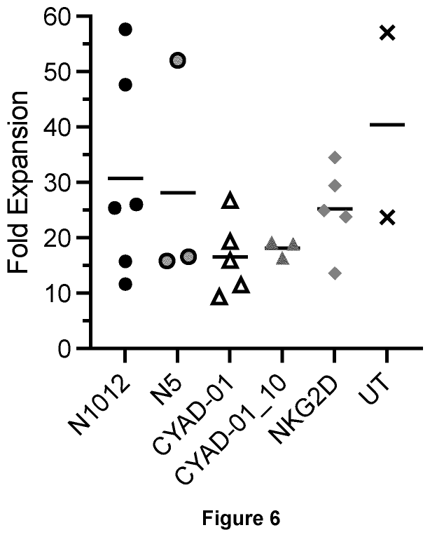

The ability to grow T-cells to therapeutically viable numbers is an important step in the translation of CAR T-cell therapies. Critically, N5 T-cells demonstrated robust growth in vitro when cultured for 14 days for use in in vivo studies (FIG. 6).

### Example 3: In Vivo Function of the N1-N5 CAR T-Cells

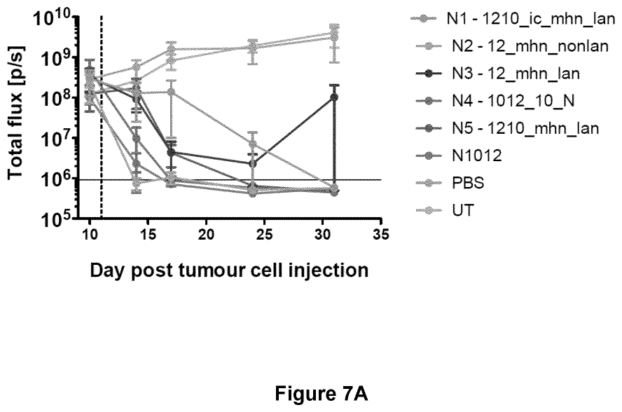

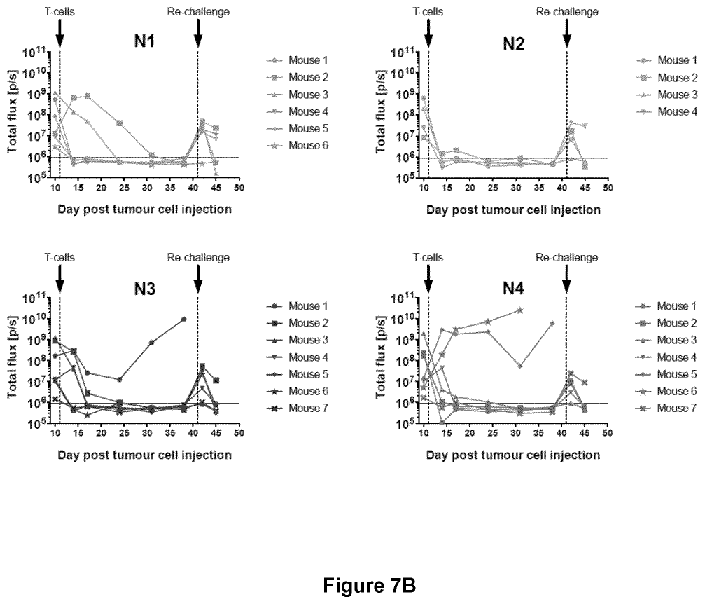

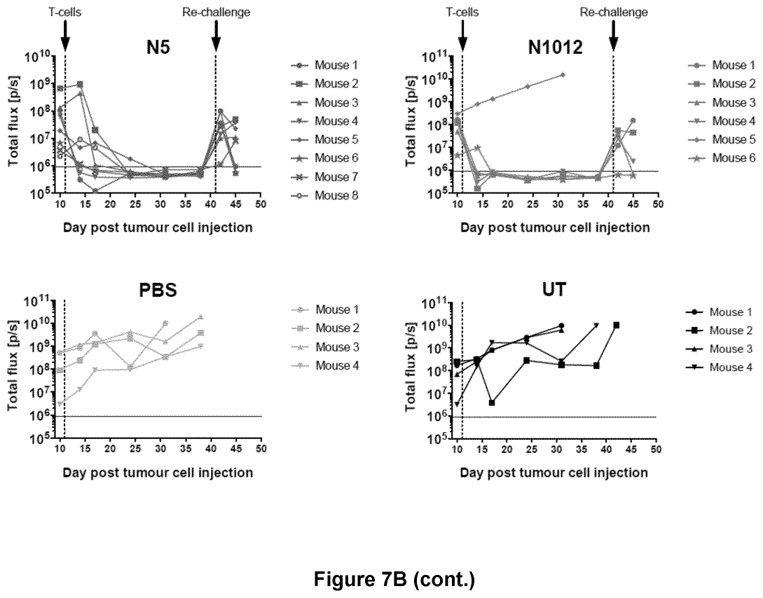

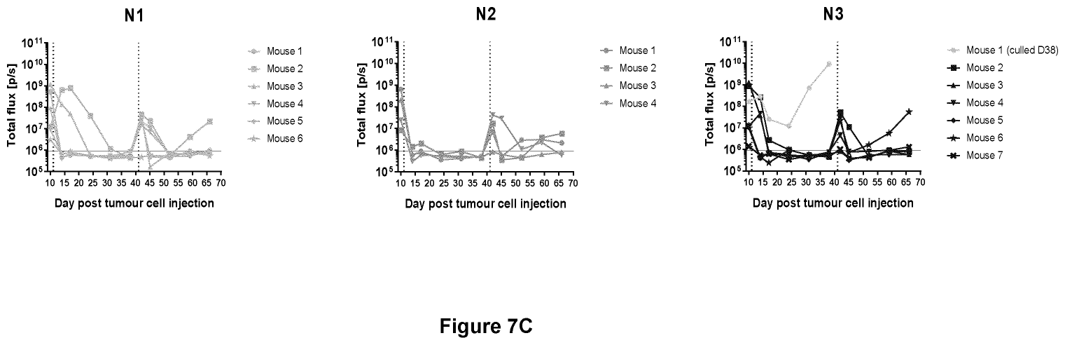

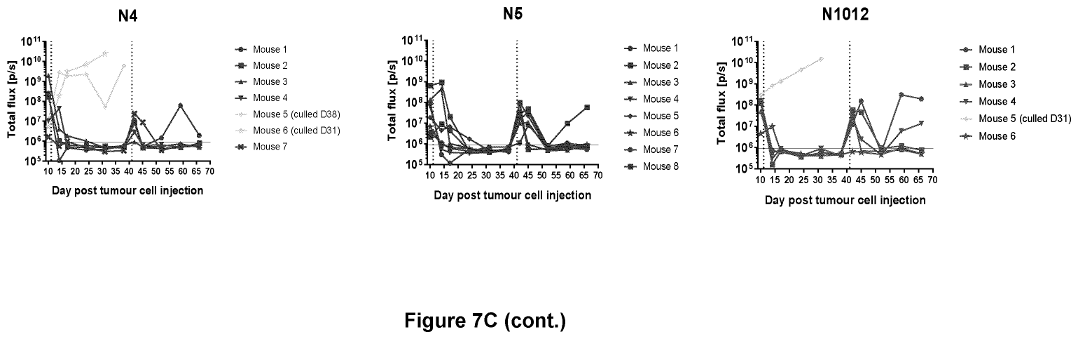

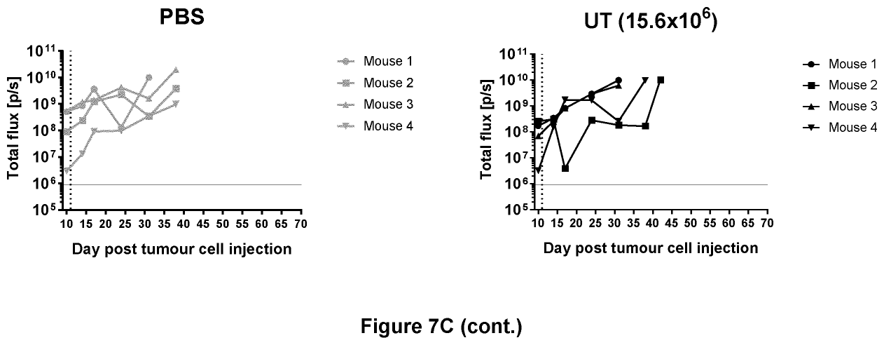

To test the efficacy of N1-N5 and N1012 CAR T-cells in vivo, 1×10⁷ CAR⁺ T-cells were injected i.p. into NSG mice twelve days after i.p. inoculation with 1×10⁵ ffLUC-tagged BxPC3 cells. Tumour growth was monitored weekly by bioluminescence imaging and the data are presented as both average total flux (photons/second) per treatment group (FIG. 7A), and total flux (photons/second) per individual mouse (FIG. 7B and FIG. 7C). Despite previous data from our lab demonstrating this mouse model to be refractory to treatment with other CAR T-cells, significant and sustained tumour regression was observed in mice treated with N1-N5 CAR T-cells. Indeed, tumour was completely eradicated in 6/6 mice treated with N1 T-cells, 4/4 mice treated with N2 T-cells, 6/7 mice treated with N3 T-cells, 5/7 treated with N4 T-cells, 8/8 treated with N5 T-cells, and 5/6 mice treated with N1012 T-cells. In contrast, the kinetics of tumour growth in mice treated with UT T-cells were identical to those receiving PBS. These data confirm that tumour eradication was CAR-specific.

To investigate the potential formation of memory T-cells, mice that were tumour free at day 41 (29 days after T-cell infusion) were re-challenged i.p. with a fresh bolus of 1×10⁵ ffLUC-tagged BxPC3 cells. Mice were imaged on Day 42 to confirm tumour take. Subsequent imaging demonstrated that 33/34 re-challenged mice rejected the tumour. These data suggest the N1-N5 CAR T-cells are able to form memory and re-activate in response to re-emergence of the target (FIG. 7B). This is further confirmed in FIG. 7C, which shows the experiment of FIG. 7B extended to 70 days.

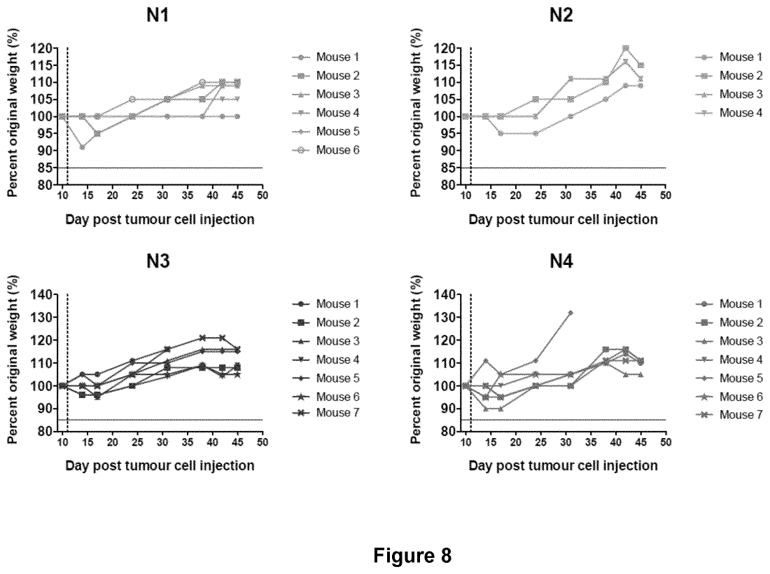

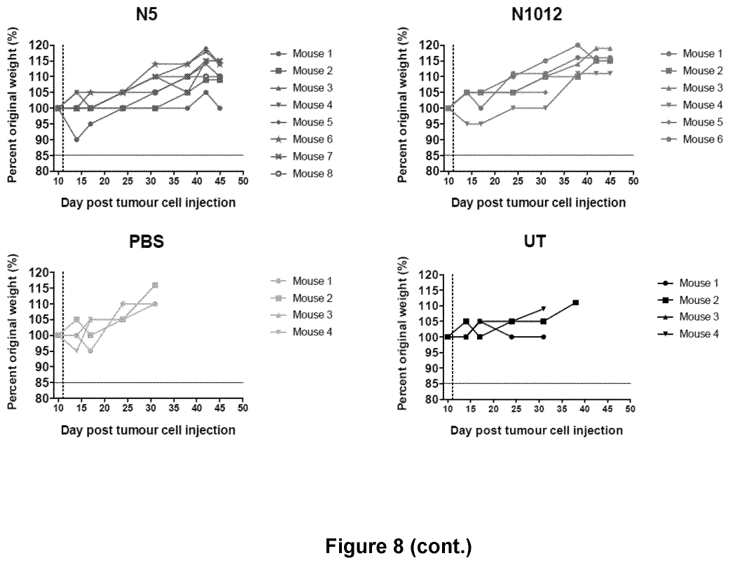

Despite such anti-tumour efficacy, no evidence of toxicity was observed when assessed by weight loss (FIG. 8).

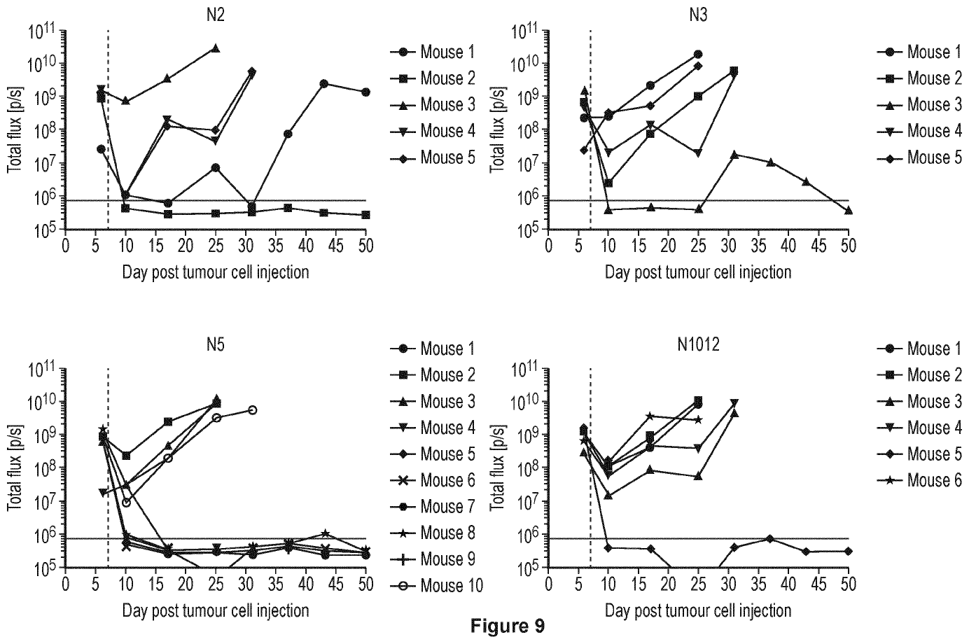

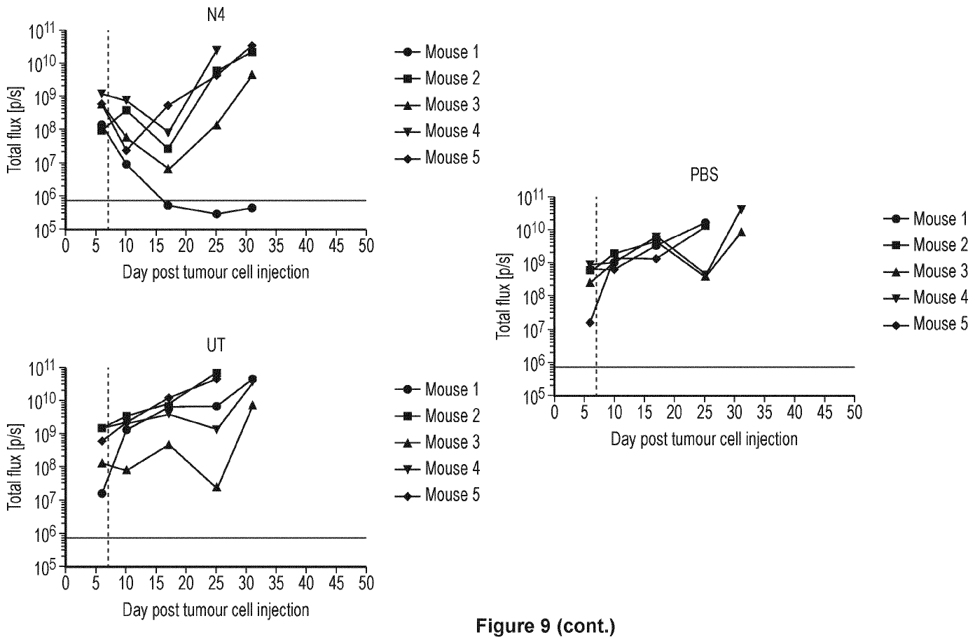

To further confirm efficacy in the same refractory model of pancreatic cancer, SCID-Beige mice were inoculated i.p. with 1×10⁶ ffLUC-tagged BxPC3 cells. Treatment with 1×10⁷ N2, N3, N4, N5 or N1012 T-cells i.p. seven days after tumour inoculation demonstrated anti-tumour efficacy with all functional T-cells. Overall, 6/10 mice that received N5 T-cells, and 1/5 mice that received N2, N3, N4 or N1012 T-cells demonstrated a complete eradication of tumour (FIG. 9). A transient dip in tumour growth was also noted in most other mice treated with N1-N5 T-cells. These effects contrasted with the lack of efficacy observed with UT T-cells, confirming the dependency of CAR activation for the anti-tumour response. The reduced efficacy observed in this SCID-Beige mouse model, compared to the NSG mouse model, likely reflects the poorer engraftment potential of human T-cells in this strain of mice. To confirm this, mice that remained tumour-free at Day 63 were re-challenged with 1×10⁶ ffLUC-tagged BxPC3 cells administered into the i.p. cavity. Overall, tumour growth was observed in 5/6 re-challenged mice, suggesting poor engraftment of human T-cells in the SCID-Beige mouse strain (FIG. 10).

### Example 4: Comparison of N5 and N1012 CAR T-Cells In Vivo

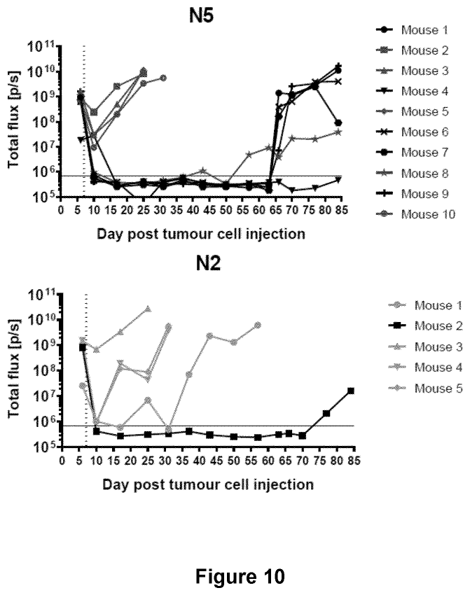

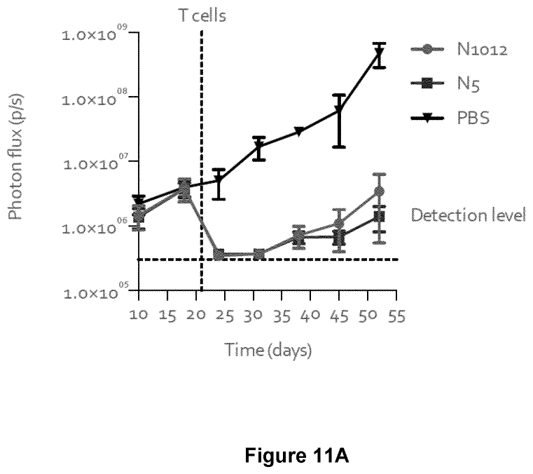

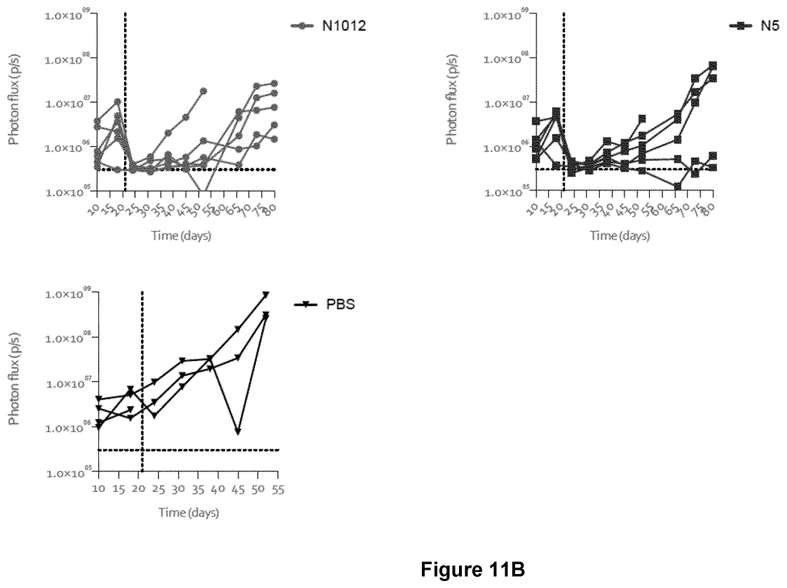

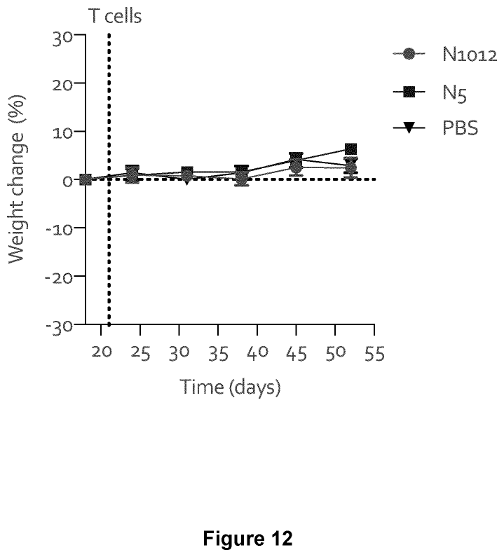

With initial data suggesting that N5 CAR T-cells mediated the most reproducible anti-tumour responses, they were compared against N1012 CAR T-cells in a model of refractory ovarian cancer. Twenty-one days after inoculation with 1×10⁶ ffLUC-tagged SKOV3 cells, SCID-Beige mice were treated i.p. with 5×10⁶ N5 T-cells, N1012 T-cells, or with PBS. Tumour growth was monitored by weekly bioluminescence imaging and the data are presented as both average total flux (photons/second) per treatment group (FIG. 11A), or total flux (photons/second) per individual mouse (FIG. 11B). A significant reduction in tumour burden was observed in those mice treated with either N5 or N1012 T-cells, further demonstrating the potent efficacy of these T-cells. In contrast, steady tumour growth was observed in mice receiving PBS. Critically, despite such potent anti-tumour effects, no evidence of toxicity was observed (FIG. 12).

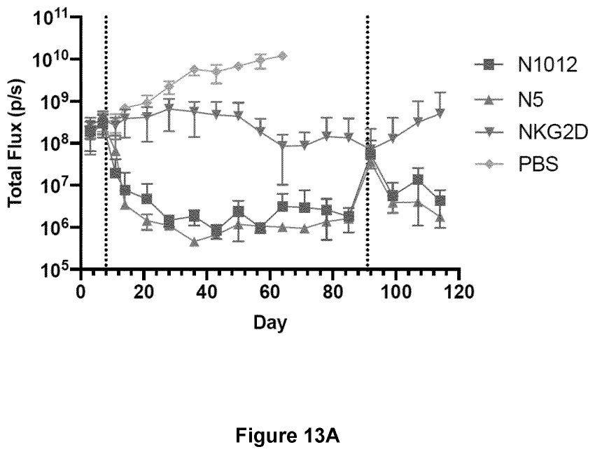

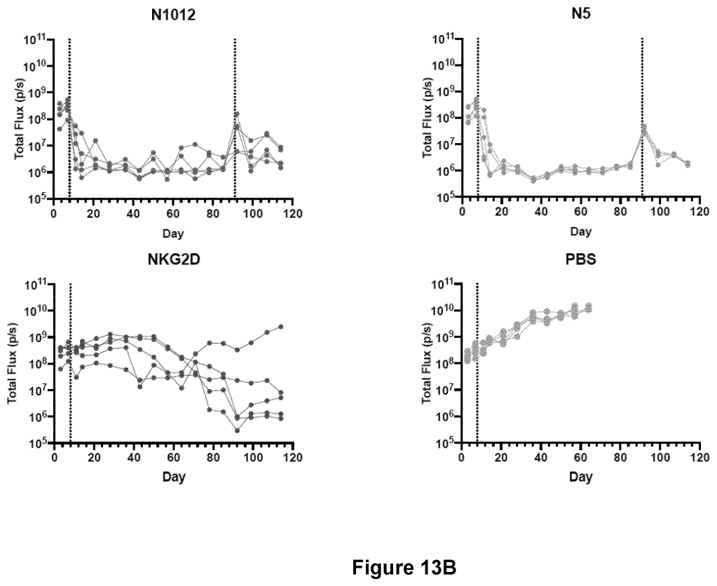

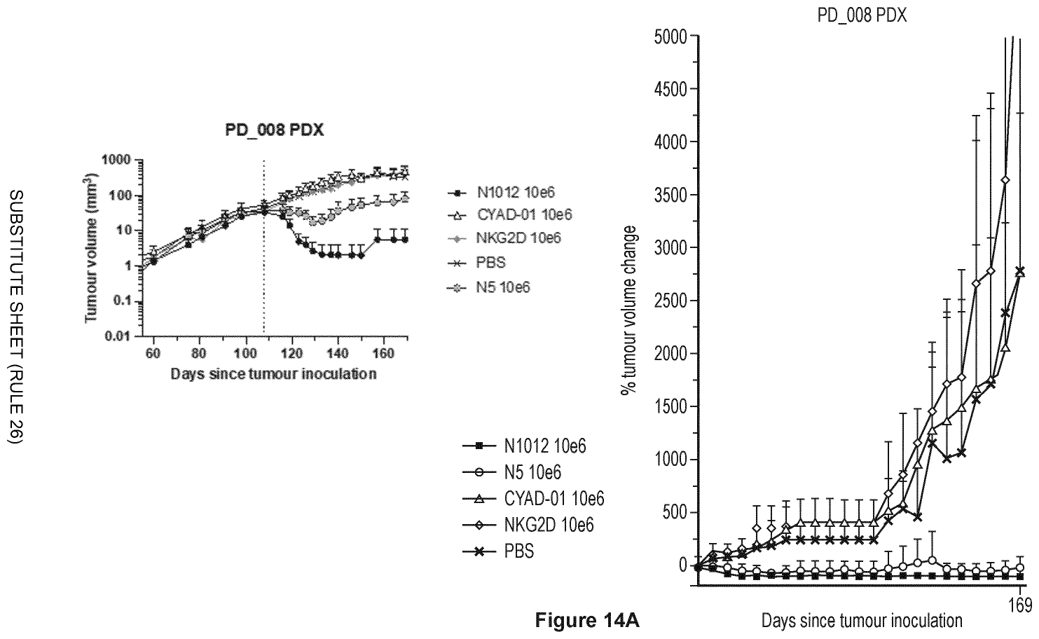

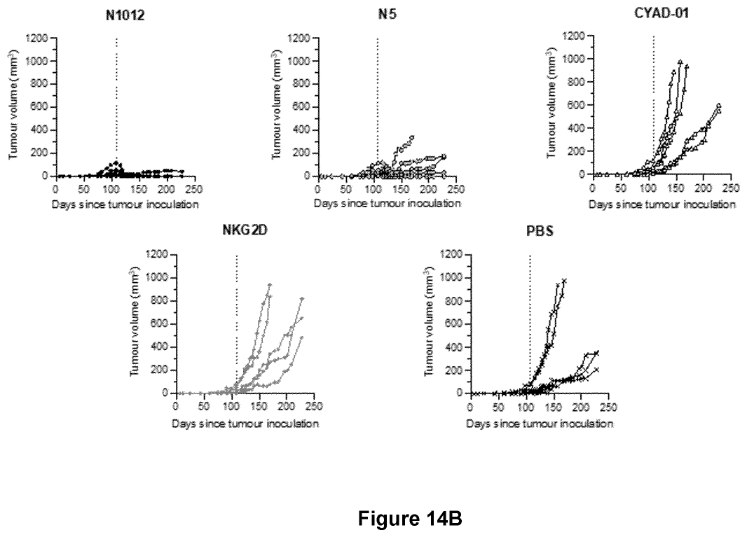

To further compare the anti-tumour efficacy of N5 and N1012 T-cells, NSG mice were inoculated i.p. with 1×10⁶ ffLUC-tagged H226 malignant mesothelioma cells. Eight days after tumour inoculation, mice were treated with either PBS, or 4×10⁶ T-cells expressing either N5, or N1012. As a control, one group of mice were treated with 4×10⁶ T-cells expressing NKG2D alone. Tumour growth was monitored weekly by bioluminescent imaging and the data are presented as average total flux (photons/second) per treatment (FIG. 13A) and as total flux (photons/second) per individual mouse (FIG. 13B). Whereas consistent tumour growth was observed in the mice that received PBS, 100% tumour eradication was observed in mice receiving N1012 or N5 T-cells.

To confirm T-cell persistence and maintenance of function, all tumour-free mice were inoculated i.p. with an additional 1×10⁶ffLUC-tagged H226 cells, 91 days after initial tumour inoculation. Tumour take was confirmed in all mice after 24 hours by bioluminescence imaging. All re-challenged mice rejected the tumour, confirming persistence of the N1012 and N5 T-cells and the ability of these T-cells to mediate long-term tumour control.

### Example 5: Comparison of N5 and CYAD-01 CAR T-Cells In Vivo

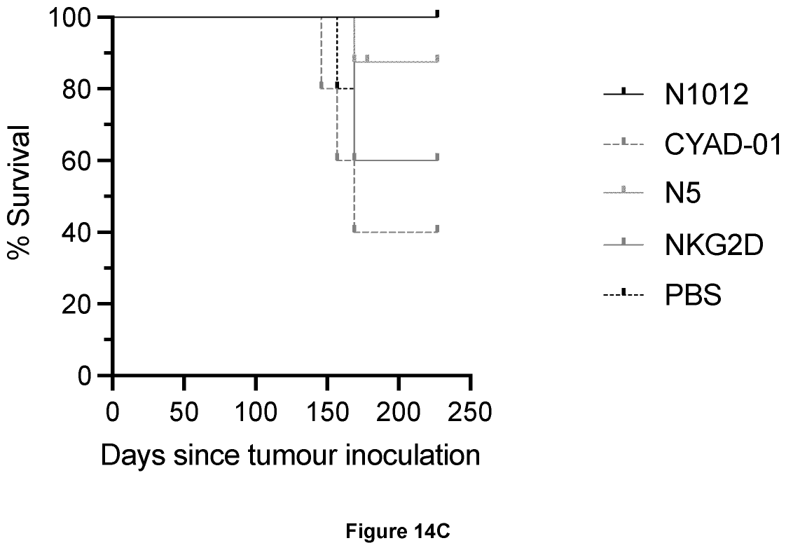

The in vivo anti-tumour response of N5 CAR T-cells was then compared to CYAD-01 CAR T-cells, N1012 and NKG2D CAR T-cells in another tumour model, through the generation of a mesothelioma patient-derived xenograft (PDX). The PDX tumour was implanted subcutaneously into the flanks of 29 NSG mice using a trocar needle. Upon reaching approximately 63 mm³ (range 4.2 mm³-189 mm³), the mice were evenly distributed across all treatment groups to ensure a comparable average tumour volume. Mice were treated intravenously on Day 108 with PBS, or with 1×10⁷ N5, N1012, or CYAD-01 CAR T-cells. As a control, one group of mice received 1×10⁷ T-cells expressing NKG2D alone. Tumour growth was monitored by weekly caliper measurements and the data are presented as average tumour volume per treatment group (FIG. 14A, left graph) and percentage change in tumour volume (FIG. 14A, right graph). The tumour volume for each individual mouse per group is presented in FIG. 14B. Rapid tumour growth was observed in those mice treated with either PBS, T-cells expressing NKG2D alone, or those expressing the CYAD-01 CAR. In contrast, a dramatic and sustained reduction in tumour burden was observed in those mice treated with either N1012 or N5 T-cells. This resulted in a significant enhancement in the survival of mice treated with either N1012 or N5 T-cells, compared to all other treatments (FIG. 14C).

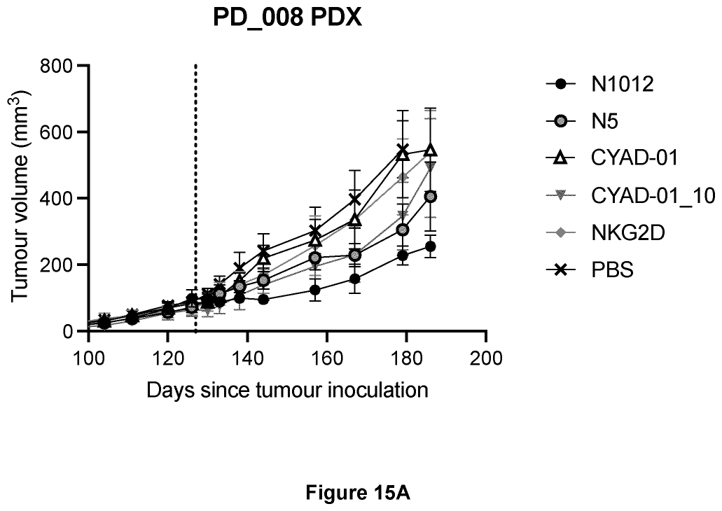

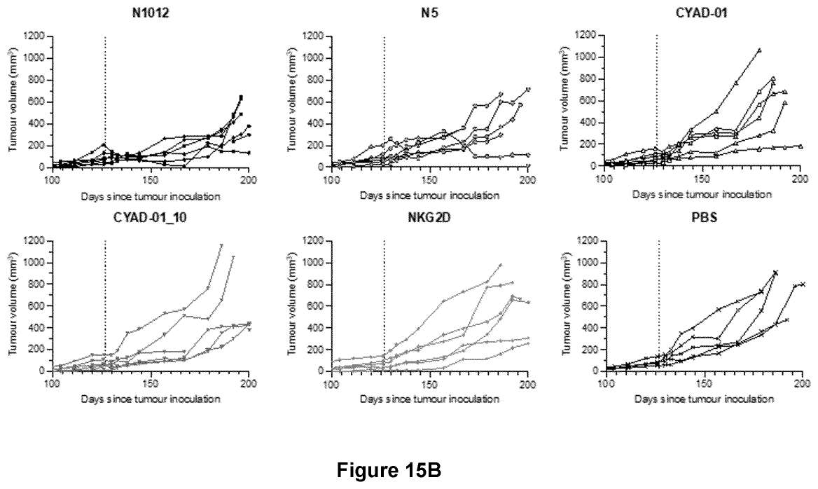

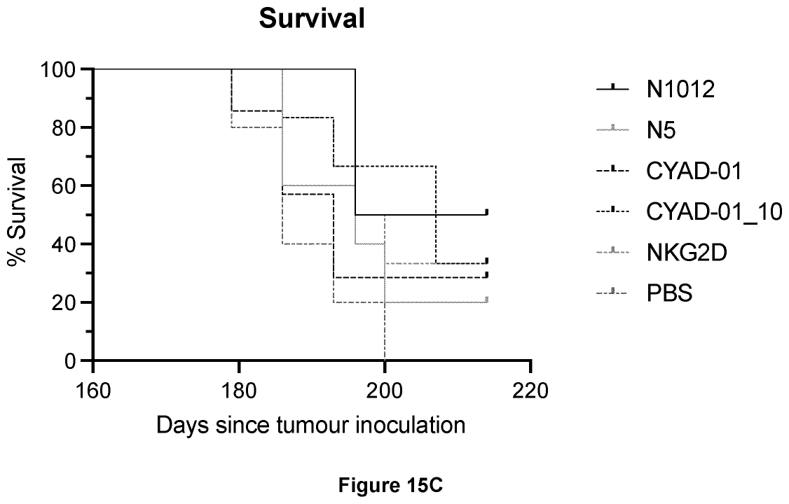

To investigate whether a similarly robust anti-tumour response could be achieved with a lower T-cell dose, a second PDX experiment was undertaken. Again, tumour chunks measuring approximately 4.2 mm³ were implanted subcutaneously into the flanks of 40 NSG mice. As before, the tumours were allowed to develop to approximately 76 mm³ (range 4.2 mm³-210 mm³) and the mice distributed into groups with a comparable average tumour burden. Mice were treated intravenously on Day 127 with either PBS, or with 4×10⁶ N5, N1012, CYAD-01 or CYAD-01_10 CAR T-cells. As a control, one group of mice received 4×10⁶ T-cells expressing NKG2D alone. As before, tumour growth was measured by weekly caliper measurement and the data are presented as average tumour volume per treatment group (FIG. 15A) and tumour volume for each individual mouse (FIG. 15B). Mice that received N1012 or N5 T-cells demonstrated a reduction in the rate of tumour growth when compared to those mice treated with either PBS, NKG2D control T-cells, CYAD-01, or CYAD-01_10 CAR T-cells. This reduction in tumour growth rate led to an improved survival of N5 T-cells compared to mice receiving PBS (FIG. 15C).

Taken together, these data confirm the potent and sustained anti-tumour efficacy mediated by N1-N5 CAR T-cells.

SEQ ID NO: 1
(mouse NKG2D)
MALIRDRKSHHSEMSKCHNYDLKPAKWDTSQEQQKQRLAL
TTSQPGENGIIRGRYPIEKLKISPMFVVRVLAIALAIRFT
LNTLMWLAIFKETFQPVLCNKEVPVSSREGYCGPCPNNWI
CHRNNCYQFFNEEKTWNQSQASCLSQNSSLLKIYSKEEQD
FLKLVKSYHWMGLVQIPANGSWQWEDGSSLSYNQLTLVEI
PKGSCAVYGSSFKAYTEDCANLNTYICMKRAV
SEQ ID NO: 2
(mouse NKG2D transmembrane domain;
UniProt accession NO: O54709)
VVRVLAIALAIRFTLNTLMWLAI
SEQ ID NO: 3
(mouse NKG2D transmembrane domain)
KISPMFVVRVLAIALAIRFTLNTLMWLAIFKETFQPV
SEQ ID NO: 4
(rat NKG2D)
MSKCHNYDLKPAKWDTSQEHQKQRSALPTSRPGENGIIRR
RSSIEELKISPLFVVRVLVAAMTIRFTVITLTWLAVFITL
LCNKEVSVSSREGYCGPCPNDWICHRNNCYQFFNENKAWN
QSQASCLSQNSSLLKIYSKEEQDFLKLVKSYHWMGLVQSP
ANGSWQWEDGSSLSPNELTLVKTPSGTCAVYGSSFKAYTE
DCSNPNTYICMKRAV
SEQ ID NO: 5
(rat NKG2D Transmembrane Domain-UniProt
accession NO: O70215 aa 52-74)
LFVVRVLVAAMTIRFTVITLTWL
SEQ ID NO: 6
(human NKG2D-UniProt accession NO: P26718)
MGWIRGRRSRHSWEMSEFHNYNLDLKKSDFSTRWQKQRCP
VVKSKCRENASPFFFCCFIAVAMGIRFIIMVAIWSAVFLN
SLFNQEVQIPLTESYCGPCPKNWICYKNNCYQFFDESKNW
YESQASCMSQNASLLKVYSKEDQDLLKLVKSYHWMGLVHI
PTNGSWQWEDGSILSPNLLTIIEMQKGDCALYASSFKGYI
ENCSTPNTYICMQRTV
SEQ ID NO: 7
(human extracellular NKG2D domain)
LFNQEVQIPLTESYCGPCPKNWICYKNNCYQFFDESKNWY
ESQASCMSQNASLLKVYSKEDQDLLKLVKSYHWMGLVHIP
TNGSWQWEDGSILSPNLLTIIEMQKGDCALYASSFKGYIE
NCSTPNTYICMQRTV
SEQ ID NO: 8
(human extracellular NKG2D domain)
IWSAVFLNSLFNQEVQIPLTESYCGPCPKNWICYKNNCYQ
FFDESKNWYESQASCMSQNASLLKVYSKEDQDLLKLVKSY
HWMGLVHIPTNGSWQWEDGSILSPNLLTIIEMQKGDCALY
ASSFKGYIENCSTPNTYICMQRTV
SEQ ID NO: 9
IWSAVFLNS
SEQ ID NO: 10
(SEQ ID NO: 7 minus 8 most
N-terminal amino acids)
PLTESYCGPCPKNWICYKNNCYQFFDESKNWYESQASCMS
QNASLLKVYSKEDQDLLKLVKSYHWMGLVHIPTNGSWQWE
DGSILSPNLLTIIEMQKGDCALYASSFKGYIENCSTPNTY
ICMQRTV
SEQ ID NO: 11
(human NKG2D intracellular)
MGWIRGRRSRHSWEMSEFHNYNLDLKKSDFSTRWQKQRCPV
VKSKCRENAS
SEQ ID NO: 12
(human NKG2D intracellular domain)
MGWIRGRRSRHSWEMSEFHNYNLDLKKSDFSTRWQKQRC
PVVKSKCRENA
SEQ ID NO: 13
(murine intracellular NKG2D domain, short
isoform-UniProt accession NO: 054709-2 aa
13-66)
MSKCHNYDLKPAKWDTSQEQQKQRLALTTSQPGENGII
RGRYPIEKLKISPMF
SEQ ID NO: 14
(murine intracellular NKG2D domain)
MSKCHNYDLKPAKWDTSQEQQKQRLALTTSQPGENGI
IRGRYPIEKL
SEQ ID NO: 15
(rat NKG2D Intracellular Domain-
UniProt accession NO: O70215 aa 1-51)
MSKCHNYDLKPAKWDTSQEHQKQRSALPTSRPGENGII
RRRSSIEELKISP
SEQ ID NO: 16
(DAP12 human polypeptide UniProt
accession NO: O43914)
MGGLEPCSRL LLLPLLLAVS GLRPVQAQAQ
SDCSCSTVSP GVLAGIVMGD LVLTVLIALA
VYFLGRLVPR GRGAAEAATR KORITETESP
YQELQGQRSD VYSDLNTQRP YYK
SEQ ID NO: 17:
(human DAP12 polypeptide-cytoplasmic/
intracellular domain aa
62-113)
YFLGRLVPRGRGAAEAATRKQRITETESPYQE
LQGQRSDVYSDLNTQRPYYK
SEQ ID NO: 18
(human DAP12 polypeptide-
transmembrane domain aa
41-61)
GVLAGIVMGD LVLTVLIALA V
SEQ ID NO: 19
(human DAP12 polypeptide aa
22-61-extracellular and transmembrane
domains)
LRPVQAQAQS DCSCSTVSPG VLAGIVMGDL
VLTVLIALAV
SEQ ID NO: 20
(human DAP12 polypeptide-
extracellular, transmembrane, and
intracellular)
LRPVQAQAQSDCSCSTVSPGVLAGIVMGDLVLTVLIALAV
YFLGRLVPRGRGAAEAATRKQRITETESPYQELQGQRSDV
YSDLNTQRPYYK
SEQ ID NO: 21
(murine (mouse) DAP12 extracellular,
transmembrane and intracellular
domains, with a leader sequence
(UniProt accession NO: O54885 aa 1-114))
MGALEPSWCLLFLPVLLTVGGLSPVQAQSDTFPRCDCSSV
SPGVLAGIVLGDLVLTLLIALAVYSLGRLVSRGQGTAEGT
RKQHIAETESPYQELQGQRPEVYSDLNTQRQYYR
SEQ ID NO: 22
(murine (mouse) DAP12 Intracellular
Domain (UniProt accession NO: O54885
aa 64-114))
YSLGRLVSRGQGTAEGTRKQHIAETESPYQELQG
QRPEVYSDLNTQRQYYR
SEQ ID NO: 23
(murine (mouse) DAP12 Transmembrane
Domain (UniProt accession NO:
O54885 aa 43-63))
GVLAGIVLGDLVLTLLIALAV
SEQ ID NO: 24
(murine (mouse) DAP12 extracellular
and transmembrane domains (UniProt
accession NO: O54885 aa 22-63))
LSPVQAQSDTFPRCDCSSVSP GV
LAGIVLGDLVLTLLIALAV
SEQ ID NO: 25
(murine (mouse) DAP12 extracellular,
intracellular, and transmembrane
domains (UniProt accession NO:
O54885 aa 22-114))
LSPVQAQSDTFPRCDCSSVSP GVLAGIVLGD
LVLTLLIALAVYSLGRLVSRGQGTAEGTRK
QHIAETESPYQELQGQRPEVYSDLNTQRQYYR
SEQ ID NO: 26
(human DAP10 UniProt accession NO: Q9UBK5)
MIHLGHILFL LLLPVAAAQT TPGERSSLPA
FYPGTSGSCS GCGSLSLPLL AGLVAADAVA
SLLIVGAVFL CARPRRSPAQ EDGKVYINMP
GRG
SEQ ID NO: 27 (human DAP10)
QTTPGERSSL PAFYPGTSGS CSGCGSLSLP
LLAGLVAADA VASLLIVGAV FLCARPRRSP
AQEDGKVYIN MPGRG
SEQ ID NO: 28
(human DAP10 intracellular domain)
LCARPRRSPAQEDGKVYINMPGRG
SEQ ID NO: 29
(human DAP10 extracellular and
transmembrane domains)
QTTPGERSSL PAFYPGTSGS CSGCGSLSLP
LLAGLVAADA VASLLIVGAV F
SEQ ID NO: 30
(amino acids 1-71 of human DAP10)
MIHLGHILFL LLLPVAAAQT TPGERSSLPA
FYPGTSGSCS GCGSLSLPLL AGLVAADAVA
SLLIVGAVFL C
SEQ ID NO: 31
(amino acids 19-71 of human DAP10)
QTTPGERSSL PAFYPGTSGS CSGCGSLSLP
LLAGLVAADA VASLLIVGAV FLC
SEQ ID NO: 32
(amino acids 49-93 of human DAP10)
LLAGLVAADA VASLLIVGAV FLCARPRRSP
AQEDGKVYIN MPGRG
SEQ ID NO: 33
(amino acids 49-69 of human DAP10)
LLAGLVAADA VASLLIVGAV F
SEQ ID NO: 34:
(mouse DAP10-UniProt accession NO: Q9QUJ0)
MDPPGYLLFLLLLPVAASQTSAGSCSGCGTLSLP
LLAGLVAADAVMSLLIVGVVFVCMRPHGRPAQED
GRVYINMPGRG
SEQ ID NO: 35
(mouse DAP10-UniProt accession NO: Q9QUJ0)
SQTSAGSCSGCGTLSLPL LAGLVAADAVMSLLIVGVVFV
CMRPHGRPAQEDGRVYINMPGRG
SEQ ID NO: 36
(murine (mouse) DAP10 Intracellular
Domain-UniProt accession NO:
Q9QUJ0 aa 57-79))
CMRPHGRPAQEDGRVYINMPGRG
SEQ ID NO: 37
(CD8a signal peptide)
MALPVTALLLPLALLLHAARP
SEQ ID NO: 38
(murine (mouse) DAP10 signal
peptide-UniProt accession NO: Q9QUJ0 aa
1-17))
MDPPGYLLFLLLLPVAA
SEQ ID NO: 39
(murine (mouse) DAP12 signal peptide)
MGALEPSWCLLFLPVLLTVGG
SEQ ID NO: 40 (human DAP12 Signal Peptide-
UniProt accession NO: O43914 aa 1-21)
MGGLEPCSRLLLLPLLLAVSG
SEQ ID NO: 41 (His tag)
HHHHHH
SEQ ID NO: 42 (FLAG tag)
DYKDDDDK
SEQ ID NO: 43 (Avi tag)
GLNDIFEAQKIEWHE
SEQ ID NO: 44 (V5 tag)
GKPIPNPLLGLDST
SEQ ID NO: 45 (V5 tag)
IPNPLLGLD
SEQ ID NO: 46 (Myc tag)
EQKLISEEDL
SEQ ID NO: 47
(human DAP12 polypeptide-
transmembrane and intracellular)
GVLAGIVMGDLVLTVLIALAVYFLGRLVPRGRGAAEAATR
KQRITETESPYQELQGQRSDVYSDLNTQRPYYK
SEQ ID NO: 48 (4-1BB endodomain)
KRGRKKLLYI FKOPFMRPVQ TTQEEDGCSC
RFPEEEEGGC EL
SEQ ID NO: 49 (CD27 endodomain)
QRRKYRSNKG ESPVEPAEPC HYSCPREEEG
STIPIQEDYR KPEPACSP
SEQ ID NO: 50 (human IgG1 hinge)
EPKSCDKTHT CP
SEQ ID NO: 51 (truncated CD8α hinge)
TTTPAPRPPT PAPTIASQPL SLRPEACRPA
AGGAVHTRGL DFACD
SEQ ID NO: 52 (SGSG linker)
SGSG
SEQ ID NO: 53 (linker)
GSGGG
SEQ ID NO: 54 (linker)
GSGG
SEQ ID NO: 55 (linker)
GPPGS
SEQ ID NO: 56 (furin cleavage site)
RRKR
SEQ ID NO: 57 (P2A skip peptide)
ATNFSLLKQAGDVEENPGP
SEQ ID NO: 58 (T2A skip peptide)
EGRGSLLTCGDVEENPGP
SEQ ID NO: 59 (SGSG + P2A)
SGSGATNFSLLKQAGDVEENPGP
SEQ ID NO: 60 (SGSG + T2A)
SGSGEGRGSLLTCGDVEENPGP
SEQ ID NO: 61 (furin + SGSG + P2A)
RRKRSGSGATNFSLLKQAGDVEENPGP
SEQ ID NO: 62 (furin + SGSG + T2A)
RRKRSGSGEGRGSLLTCGDVEENPGP
SEQ ID NO: 63 (F2A skip peptide)
VKQTLNFDLLKLAGDVESNPGP
SEQ ID NO: 64 (E2A skip peptide)
QCTNYALLKLAGDVESNPGP
SEQ ID NO: 65
(SGSG linker + P2A ribosomal skip
peptide + methionine)
SGSGATNFSLLKQAGDVEENPGPM
SEQ ID NO: 66 (N1 polypeptide)
MALPVTALLLPLALLLHAARPDYKDDDDKLRPVQAQAQSD
CSCSTVSPGVLAGIVMGDLVLTVLIALAVYFLGRLVPRGR
GAAEAATRKQRITETESPYQELQGQRSDVYSDLNTQRPYY
KRRKRSGSGATNFSLLKQAGDVEENPGPMLCARPRRSPAQ
EDGKVYINMPGRGKISPMFVVRVLAIALAIRFTLNTLMWL
AIFKETFQPVLFNQEVQIPLTESYCGPCPKNWICYKNNCY
QFFDESKNWYESQASCMSQNASLLKVYSKEDQDLLKLVKS
YHWMGLVHIPTNGSWQWEDGSILSPNLLTIIEMQKGDCAL
YASSFKGYIENCSTPNTYICMQRTV
SEQ ID NO: 67 (N2 polypeptide)
MALPVTALLLPLALLLHAARPDYKDDDDKLRPVQAQAQSD
CSCSTVSPGVLAGIVMGDLVLTVLIALAVYFLGRLVPRGR
GAAEAATRKQRITETESPYQELQGQRSDVYSDLNTQRPYY
KRRKRSGSGATNFSLLKQAGDVEENPGPMVVRVLAIALAI
RFTLNTLMWLAIIWSAVFLNSLFNQEVQIPLTESYCGPCP
KNWICYKNNCYQFFDESKNWYESQASCMSQNASLLKVYSK
EDQDLLKLVKSYHWMGLVHIPTNGSWQWEDGSILSPNLLT
IIEMQKGDCALYASSFKGYIENCSTPNTYICMQRTV
SEQ ID NO: 68 (N3 polypeptide)
MALPVTALLLPLALLLHAARPDYKDDDDKLRPVQAQAQSD
CSCSTVSPGVLAGIVMGDLVLTVLIALAVYFLGRLVPRGR
GAAEAATRKQRITETESPYQELQGQRSDVYSDLNTQRPYY
KRRKRSGSGATNFSLLKQAGDVEENPGPMKISPMFVVRVL
AIALAIRFTLNTLMWLAIFKETFQPVLFNQEVQIPLTESY
CGPCPKNWICYKNNCYQFFDESKNWYESQASCMSQNASLL
KVYSKEDQDLLKLVKSYHWMGLVHIPTNGSWQWEDGSILS
PNLLTIIEMQKGDCALYASSFKGYIENCSTPNTYICMQRT
V
SEQ ID NO: 69 (N5 polypeptide)
MALPVTALLLPLALLLHAARPDYKDDDDKLRPVQAQAQSD
CSCSTVSPGVLAGIVMGDLVLTVLIALAVYFLGRLVPRGR
GAAEAATRKQRITETESPYQELQGQRSDVYSDLNTQRPYY
KRRKRSGSGEGRGSLLTCGDVEENPGPMIHLGHILFLLLL
PVAAAQTTPGERSSLPAFYPGTSGSCSGCGSLSLPLLAGL
VAADAVASLLIVGAVFLCARPRRSPAQEDGKVYINMPGRG
RRKRSGSGATNFSLLKQAGDVEENPGPMKISPMFVVRVLA
IALAIRFTLNTLMWLAIFKETFQPVLFNQEVQIPLTESYC
GPCPKNWICYKNNCYQFFDESKNWYESQASCMSQNASLLK
VYSKEDQDLLKLVKSYHWMGLVHIPTNGSWQWEDGSILSP
NLLTIIEMQKGDCALYASSFKGYIENCSTPNTYICMQRTV
SEQ ID NO: 70 (N1 DNA)
ATGGCTCTGCCTGTGACAGCTCTGCTGCTGCCTCTGGCTC
TGCTGCTGCACGCCGCTAGACCCGATTATAAGGACGACGA
CGACAAGCTGAGACCCGTGCAGGCCCAGGCCCAGAGCGAC
TGCAGCTGCAGCACCGTGAGCCCCGGCGTGCTGGCCGGCA
TCGTGATGGGCGACCTGGTGCTGACCGTGCTCATCGCCCT
TGCCGTGTACTTCCTGGGCAGACTGGTCCCCAGGGGCAGA
GGAGCTGCCGAGGCCGCTACCAGAAAGCAGAGGATCACCG
AGACAGAGAGCCCCTACCAGGAGCTGCAGGGCCAGAGATC
CGACGTGTACAGCGACCTCAACACCCAGAGACCCTATTAC
AAGAGGCGGAAGCGCTCCGGGAGTGGGGCTACCAATTTCT
CTCTCCTCAAGCAAGCCGGAGACGTTGAGGAAAACCCTGG
ACCCATGCTGTGCGCCAGGCCCAGGCGGAGCCCTGCCCAG
GAGGACGGCAAGGTGTACATCAACATGCCCGGCCGGGGCA
AAATATCTCCAATGTTCGTTGTTCGAGTCCTTGCTATAGC
CTTGGCAATTCGATTCACCCTTAACACATTGATGTGGCTT
GCCATTTTCAAAGAGACGTTTCAGCCAGTACTGTTCAACC
AGGAGGTGCAGATCCCCCTGACCGAGAGCTACTGCGGCCC
CTGCCCAAAAAATTGGATCTGCTACAAGAACAACTGCTAC
CAGTTCTTCGACGAGAGCAAGAACTGGTACGAGAGCCAGG
CCAGCTGCATGAGCCAGAACGCCAGCCTGCTGAAGGTGTA
CAGCAAGGAGGACCAGGACCTGCTGAAGCTGGTGAAGAGC
TACCACTGGATGGGCCTGGTGCACATCCCCACCAACGGCA
GCTGGCAGTGGGAGGACGGCAGCATCCTGAGCCCCAACCT
GCTGACCATCATCGAGATGCAGAAGGGCGACTGCGCCCTG
TACGCCAGCAGCTTCAAGGGCTACATCGAGAACTGCAGCA
CCCCCAACACCTACATCTGCATGCAGCGGACCGTG
SEQ ID NO: 71 (N2 DNA)
ATGGCTCTGCCTGTGACAGCTCTGCTGCTGCCTCTGGCTC
TGCTGCTGCACGCCGCTAGACCCGATTATAAGGACGACGA
CGACAAGCTGAGACCCGTGCAGGCCCAGGCCCAGAGCGAC
TGCAGCTGCAGCACCGTGAGCCCCGGCGTGCTGGCCGGCA
TCGTGATGGGCGACCTGGTGCTGACCGTGCTCATCGCCCT
TGCCGTGTACTTCCTGGGCAGACTGGTCCCCAGGGGCAGA
GGAGCTGCCGAGGCCGCTACCAGAAAGCAGAGGATCACCG
AGACAGAGAGCCCCTACCAGGAGCTGCAGGGCCAGAGATC
CGACGTGTACAGCGACCTCAACACCCAGAGACCCTATTAC
AAGAGGCGGAAGCGCTCCGGGAGTGGGGCTACCAATTTCT
CTCTCCTCAAGCAAGCCGGAGACGTTGAGGAAAACCCTGG
ACCCATGGTTGTTCGAGTCCTTGCTATAGCCTTGGCAATT
CGATTCACCCTTAACACATTGATGTGGCTTGCCATTATCT
GGAGCGCCGTGTTCCTGAACAGCCTGTTCAACCAGGAGGT
GCAGATCCCCCTGACCGAGAGCTACTGCGGCCCCTGCCCA
AAAAATTGGATCTGCTACAAGAACAACTGCTACCAGTTCT
TCGACGAGAGCAAGAACTGGTACGAGAGCCAGGCCAGCTG
CATGAGCCAGAACGCCAGCCTGCTGAAGGTGTACAGCAAG
GAGGACCAGGACCTGCTGAAGCTGGTGAAGAGCTACCACT
GGATGGGCCTGGTGCACATCCCCACCAACGGCAGCTGGCA
GTGGGAGGACGGCAGCATCCTGAGCCCCAACCTGCTGACC
ATCATCGAGATGCAGAAGGGCGACTGCGCCCTGTACGCCA
GCAGCTTCAAGGGCTACATCGAGAACTGCAGCACCCCCAA
CACCTACATCTGCATGCAGCGGACCGTG
SEQ ID NO: 72 (N3 DNA)
ATGGCTCTGCCTGTGACAGCTCTGCTGCTGCCTCTGGCTC
TGCTGCTGCACGCCGCTAGACCCGATTATAAGGACGACGA
CGACAAGCTGAGACCCGTGCAGGCCCAGGCCCAGAGCGAC
TGCAGCTGCAGCACCGTGAGCCCCGGCGTGCTGGCCGGCA
TCGTGATGGGCGACCTGGTGCTGACCGTGCTCATCGCCCT
TGCCGTGTACTTCCTGGGCAGACTGGTCCCCAGGGGCAGA
GGAGCTGCCGAGGCCGCTACCAGAAAGCAGAGGATCACCG
AGACAGAGAGCCCCTACCAGGAGCTGCAGGGCCAGAGATC
CGACGTGTACAGCGACCTCAACACCCAGAGACCCTATTAC
AAGAGGCGGAAGCGCTCCGGGAGTGGGGCTACCAATTTCT
CTCTCCTCAAGCAAGCCGGAGACGTTGAGGAAAACCCTGG
ACCCATGAAAATATCTCCAATGTTCGTTGTTCGAGTCCTT
GCTATAGCCTTGGCAATTCGATTCACCCTTAACACATTGA
TGTGGCTTGCCATTTTCAAAGAGACGTTTCAGCCAGTACT
GTTCAACCAGGAGGTGCAGATCCCCCTGACCGAGAGCTAC
TGCGGCCCCTGCCCAAAAAATTGGATCTGCTACAAGAACA
ACTGCTACCAGTTCTTCGACGAGAGCAAGAACTGGTACGA
GAGCCAGGCCAGCTGCATGAGCCAGAACGCCAGCCTGCTG
AAGGTGTACAGCAAGGAGGACCAGGACCTGCTGAAGCTGG
TGAAGAGCTACCACTGGATGGGCCTGGTGCACATCCCCAC
CAACGGCAGCTGGCAGTGGGAGGACGGCAGCATCCTGAGC
CCCAACCTGCTGACCATCATCGAGATGCAGAAGGGCGACT
GCGCCCTGTACGCCAGCAGCTTCAAGGGCTACATCGAGAA
CTGCAGCACCCCCAACACCTACATCTGCATGCAGCGGACC
GTG
SEQ ID NO: 73 (N5 DNA)
ATGGCTCTGCCTGTGACAGCTCTGCTGCTGCCTCTGGCTC
TGCTGCTGCACGCCGCTAGACCCGATTATAAGGACGACGA
CGACAAGCTGAGACCCGTGCAGGCCCAGGCCCAGAGCGAC
TGCAGCTGCAGCACCGTGAGCCCCGGCGTGCTGGCCGGCA
TCGTGATGGGCGACCTGGTGCTGACCGTGCTCATCGCCCT
TGCCGTGTACTTCCTGGGCAGACTGGTCCCCAGGGGCAGA
GGAGCTGCCGAGGCCGCTACCAGAAAGCAGAGGATCACCG
AGACAGAGAGCCCCTACCAGGAGCTGCAGGGCCAGAGATC
CGACGTGTACAGCGACCTCAACACCCAGAGACCCTATTAC
AAGAGGCGGAAGCGCTCCGGCTCCGGCGAGGGCCGCGGCA
GCCTGCTGACCTGCGGCGACGTGGAAGAGAACCCCGGACC
CATGATCCACCTGGGCCACATCCTGTTCCTGCTGCTGCTG
CCCGTGGCCGCTGCCCAAACAACACCCGGCGAGAGATCCT
CCTTGCCCGCTTTCTATCCCGGAACATCCGGAAGCTGTTC
CGGATGTGGATCCCTTTCTTTGCCTTTGCTTGCTGGATTG
GTCGCAGCTGACGCTGTCGCTTCCCTCCTTATTGTCGGAG
CTGTCTTCCTGTGCGCCAGGCCCAGGCGGAGCCCTGCCCA
GGAGGACGGCAAGGTGTACATCAACATGCCCGGCCGGGGC
AGGCGGAAGCGCTCCGGGAGTGGGGCTACCAATTTCTCTC
TCCTCAAGCAAGCCGGAGACGTTGAGGAAAACCCTGGACC
CATGAAAATATCTCCAATGTTCGTTGTTCGAGTCCTTGCT
ATAGCCTTGGCAATTCGATTCACCCTTAACACATTGATGT
GGCTTGCCATTTTCAAAGAGACGTTTCAGCCAGTACTGTT
CAACCAGGAGGTGCAGATCCCCCTGACCGAGAGCTACTGC
GGCCCCTGCCCAAAAAATTGGATCTGCTACAAGAACAACT
GCTACCAGTTCTTCGACGAGAGCAAGAACTGGTACGAGAG
CCAGGCCAGCTGCATGAGCCAGAACGCCAGCCTGCTGAAG
GTGTACAGCAAGGAGGACCAGGACCTGCTGAAGCTGGTGA
AGAGCTACCACTGGATGGGCCTGGTGCACATCCCCACCAA
CGGCAGCTGGCAGTGGGAGGACGGCAGCATCCTGAGCCCC
AACCTGCTGACCATCATCGAGATGCAGAAGGGCGACTGCG
CCCTGTACGCCAGCAGCTTCAAGGGCTACATCGAGAACTG
CAGCACCCCCAACACCTACATCTGCATGCAGCGGACCGTG
SEQ ID NO: 74 (N1012 polypeptide)
MIHLGHILFLLLLPVAAAQTTPGERSSLPAFYPGTSGSCS
GCGSLSLPLLAGLVAADAVASLLIVGAVFLCARPRRSPAQ
EDGKVYINMPGRGYFLGRLVPRGRGAAEAATRKQRITETE
SPYQELQGQRSDVYSDLNTQRPYYKRRKRSGSGATNFSLL
KQAGDVEENPGPMGWIRGRRSRHSWEMSEFHNYNLDLKKS
DFSTRWQKQRCPVVKSKCRENASPFFFCCFIAVAMGIRFI
IMVAIWSAVFLNSLFNQEVQIPLTESYCGPCPKNWICYKN
NCYQFFDESKNWYESQASCMSQNASLLKVYSKEDQDLLKL
VKSYHWMGLVHIPTNGSWQWEDGSILSPNLLTIIEMQKGD
CALYASSFKGYIENCSTPNTYICMQRTV
SEQ ID NO: 75 (N1012 DNA)
ATGATCCACCTGGGCCACATCCTGTTCCTGCTGCTGCTGC
CCGTGGCCGCTGCCCAGACCACCCCTGGCGAGCGGAGCAG
CCTGCCTGCCTTCTACCCTGGCACCAGCGGCAGCTGCAGC
GGCTGCGGCAGCCTGAGCCTGCCCCTGCTGGCCGGCCTGG
TGGCCGCCGACGCCGTGGCCAGCCTGCTGATCGTGGGCGC
CGTGTTCCTGTGCGCCAGGCCCAGGCGGAGCCCtGCCCAG
GAGGACGGCAAGGTGTACATCAACATGCCCGGCC
GGGGCTACTTCCTGGGCAGGCTGGTGCCCAGGGGCAGGGG
CGCTGCCGAGGCTGCCACCCGGAAGCAGCGGATCACCGAG
ACCGAGAGCCCCTACCAGGAGCTGCAGGGCCAGCGGAGCG
ACGTGTACAGCGACCTGAACACCCAGAGGCCCTACTACAA
GAGGCGGAAAAGGTCTGGGAGTGGGGCTACCAATTTCTCT
CTCCTCAAGCAAGCCGGAGACGTTGAGGAAAACCCTGGaC
CCATGGGCTGGATCCGGGGACGGAGGAGCCGGCACAGCT
GGGAGATGAGCGAGTTCCACAACTACAACCTGGACCTGAA
GAAGAGCGACTTCAGCACCCGGTGGCAGAAGCAGCGGTGC
CCCGTGGTGAAGAGCAAGTGCCGGGAGAACGCCAGCCCCT
TCTTCTTCTGCTGCTTCATCGCCGTGGCtATGGGCATCCGG
TTCATCATCATGGTGGCCATCTGGAGCG
CCGTGTTCCTGAACAGCCTGTTCAACCAGGAGGTGCAGAT
CCCCCTGACCGAGAGCTACTGCGGCCCCTGCCCCAAGAAC
TGGATCTGCTACAAGAACAACTGCTACCAGTTCTTCGACG
AGAGCAAGAACTGGTACGAGAGCCAGGCCAGCTGCATGAG
CCAGAACGCCAGCCTGCTGAAGGTGTACAGCAAGGAGGAC
CAGGACCTGCTGAAGCTGGTGAAGAGCTACCACTGGATGG
GCCTGGTGCACATCCCCACCAACGGCAGCTGGCAGTGGGA
GGACGGCAGCATCCTGAGCCCCAACCTGCTGACCATCATC
GAGATGCAGAAGGGCGACTGCGCCCTGTACGCCAGCAGCT
TCAAGGGCTACATCGAGAACTGCAGCACCCCCAACACCTA
CATCTGCATGCAGCGGACCGTG
SEQ ID NO: 76 (N4 polypeptide)
MALPVTALLLPLALLLHAARPDYKDDDDKQTTPGERSSLP
AFYPGTSGSCSGCGSLSLPLLAGLVAADAVASLLIVGAVF
YFLGRLVPRGRGAAEAATRKQRITETESPY
QELQGQRSDVYSDLNTQRPYYKRRKRSGSGEGRGSLLTCG
DVEENPGPMIHLGHILFLLLLPVAAAQTTPGERSSLPAFY
PGTSGSCSGCGSLSLPLLAGLVAADAVASLLIVGAVFLCA
RPRRSPAQEDGKVYINMPGRGRRKRSGSGATNFSLLKQAG
DVEENPGPMGWIRGRRSRHSWEMSEFHNYNLDLKKSDFST
RWQKQRCPVVKSKCRENASPFFFCCFIAVAMGIRFIIMVA
IWSAVFLNSLFNQEVQIPLTESYCGPCPKNWICYKNNCYQ
FFDESKNWYESQASCMSQNASLLKVYSKEDQDLLKLVKSY
HWMGLVHIPTNGSWQWEDGSILSPNLLTIIEMQKGDCALY
ASSFKGYIENCSTPNTYICMQRTV
SEQ ID NO: 77 (N4 DNA)
ATGGCTCTGCCTGTGACAGCTCTGCTGCTGCCTCTGGCTC
TGCTGCTGCACGCCGCTAGACCCGATTATAAGGACGACGA
CGACAAGCAGACCACCCCTGGCGAGCGGAGCAGCCTGCCT
GCCTTCTACCCTGGCACCAGCGGCAGCTGCAGCGGCTGCG
GCAGCCTGAGCCTGCCCCTGCTGGCtGGCCTGGTGGCCGC
CGACGCCGTGGCCAGCCTGCTGATCG
TGGGCGCCGTGTTCTACTTCCTGGGCAGGCTGGTGCCCAG
GGGCAGGGGCGCTGCCGAGGCTGCCACCCGGAAGCAGCGG
ATCACCGAGACCGAGAGCCCCTACCAGGAGCTGCAGGGCC
AGCGGAGCGACGTGTACAGCGACCTGAACACCCAGAGGCC
CTACTACAAGCGGAGAAAGCGCtccGGCTCCGGCGAGGGC
cgcGGCAGCCTGCTGACCTGCGGCGACGTGGAAGAGAACC
CCG
GACCCATGATCCACCTGGGCCACATCCTGTTCCTGCTGCT
GCTGCCCGTGGCCGCTGCCCAAACAACACCCGGCGAGAGA
TCCTCCTTGCCCGCTTTCTATCCCGGAACATCCGGAAGCT
GTtccggaTGTGGATCCCTTTCTTTGcctttgCTTGCTGGA
TTGGTCGCAGCTGACGCTGTCGCTTCCCTCC
TTATTGTCGGAGCTGTCTTCCTGTGCGCCAGGCCCAGGCG
GAGCCCtGCCCAGGAGGACGGCAAGGTGTACATCAACATG
CCCGGCC
GGGGCAGGCGGaagcgctccGGGAGTGGGGCTACCAATTT
CTCTCTCCTCAAGCAAGCCG
GAGACGTTGAGGAAAACCCTGGaCCcATGGGCTGGATCCG
GGGACGGAGGAGCCGGCACAGCTGGG
AGATGAGCGAGTTCCACAACTACAACCTGGACCTGAAGAA
GAGCGACTTCAGCACCCGGTGGCAGAAGCAGCGGTGCCCC
GTGGTGAAGAGCAAGTGCCGGGAGAACGCCAGCCCCTTCT
TCTTCTGCTGCTTCATCGCCGTGGCtATGGGCATCCGGTT
CATCATCATGGTGGCCATCTGGAGCG
CCGTGTTCCTGAACAGCCTGTTCAACCAGGAGGTGCAGAT
CCCCCTGACCGAGAGCTACTGCGGCCCCTGCCCCAAGAAC
TGGATCTGCTACAAGAACAACTGCTACCAGTTCTTCGACG
AGAGCAAGAACTGGTACGAGAGCCAGGCCAGCTGCATGAG
CCAGAACGCCAGCCTGCTGAAGGTGTACAGCAAGGAGGAC
CAGGACCTGCTGAAGCTGGTGAAGAGCTACCACTGGATGG
GCCTGGTGCACATCCCCACCAACGGCAGCTGGCAGTGGGA
GGACGGCAGCATCCTGAGCCCCAACCTGCTGACCATCATC
GAGATGCAGAAGGGCGACTGCGCCCTGTACGCCAGCAGCT
TCAAGGGCTACATCGAGAACTGCAGCACCCCCAACACCTA
CATCTGCATGCAGCGGACCGTG
SEQ ID NO: 78
(human DAP10 extracellular domain)
QTTPGERSSL PAFYPGTSGS CSGCGSLSLP

What is claimed is:

1. An immunoresponsive cell comprising a chimeric NKG2D protein; wherein the immunoresponsive cell is a T-cell, natural killer (NK) cell, macrophage or neutrophil and the chimeric NKG2D protein comprises a human NKG2D extracellular domain or a variant thereof and a murine NKG2D transmembrane domain or a variant thereof.

2. (canceled)

3. The immunoresponsive cell according to claim 1, wherein the chimeric NKG2D protein comprises a murine NKG2D transmembrane domain, wherein the murine NKG2D transmembrane domain is a mouse NKG2D transmembrane domain comprising an amino acid sequence selected from SEQ ID NO: 2 or 3, or a rat NKG2D transmembrane domain comprising an amino acid sequence selected from SEQ ID NO: 4 or 5.

4-7. (canceled)

8. The immunoresponsive cell according to claim 1, wherein the chimeric NKG2D protein comprises a variant of the murine NKG2D transmembrane domain, wherein the variant (i) has at least 70%, 75%, 80%, 85%, 90%, 95%, 97% or 99% sequence identity to any of SEQ ID NOs:2-5 or (ii) comprises a peptide comprising one or more point (i.e. 1, 2, 3, 4, 5, 6, 7, 8, 9, 10 or more) mutations that add, delete or substitute any of the amino acids compared any of SEQ ID NOs:2-5.

9. The immunoresponsive cell according to claim 1,
wherein the chimeric NKG2D protein comprises a human NKG2D extracellular domain, wherein the human NKG2D extracellular domain comprises an amino acid sequence selected from any one SEQ ID NOs: 7, 8 and 10.

10. (canceled)

11. The immunoresponsive cell according to claim 1, wherein the chimeric NKG2D protein comprises a variant of the human NKG2D extracellular domain, wherein the variant (i) has at least 70%, 75%, 80%, 85%, 90%, 95%, 97% or 99% sequence identity to any of SEQ ID NOs: 7, 8 and 10 or (ii) comprises a peptide comprising one or more point (i.e. 1, 2, 3, 4, 5, 6, 7, 8, 9, 10 or more) mutations that add, delete or substitute any of the amino acids compared to any of SEQ ID NOs: 7, 8 and 10.

12. The immunoresponsive cell according to claim 1, wherein the chimeric NKG2D protein comprises from N terminus to C terminus the murine NKG2D transmembrane domain or a variant thereof and a human NKG2D extracellular domain or a variant thereof.

13. The immunoresponsive cell according to claim 1, wherein the chimeric NKG2D protein does not comprise an NKG2D intracellular domain.

14. The immunoresponsive cell according to claim 1, wherein the chimeric NKG2D protein further comprises an intracellular NKG2D domain or a variant thereof in the N-terminal to the murine NKG2D transmembrane domain.

15. (canceled)

16. The immunoresponsive cell according to claim 14, wherein the intracellular NKG2D domain comprises a sequence selected from any one of SEQ ID Nos: 11-15.

17-20. (canceled)

21. The immunoresponsive cell according to claim 14, wherein the chimeric NKG2D protein comprises a variant of the intracellular NKG2D domain, wherein the variant (i) has at least 70%, 75%, 80%, 85%, 90%, 95%, 97% or 99% sequence identity to any of SEQ ID NOs:11-15 or (ii) comprises a peptide comprising one or more point (i.e. 1, 2, 3, 4, 5, 6, 7, 8, 9, 10 or more) mutations that add, delete or substitute any of the amino acids compared to any of SEQ ID NOs:11-15.

22. The immunoresponsive cell according to claim 1, wherein the cell further comprises at least one DNAX-activating protein 12 (DAP12) polypeptide or a variant thereof.

23-36. (canceled)

37. The immunoresponsive cell according to claim 1, wherein the cell further comprises at least one DNAX-activating protein 10 (DAP10) polypeptide or variant thereof.

38-49. (canceled)

50. The immunoresponsive cell according to claim 1, wherein the cell further comprises at least one DAP12 polypeptide or variant thereof fused to at least one DNAX-activating protein 10 (DAP10) polypeptide or variant thereof.

51. The immunoresponsive cell according to claim 1, wherein the cell comprises a polypeptide sequence encoding the chimeric NKG2D protein, wherein the polypeptide sequence comprises any one of SEQ ID NOs:66-69.

52. The immunoresponsive cell according to claim 1, wherein the cell comprises a polypeptide sequence encoding the chimeric NKG2D protein, wherein the polypeptide sequence has at least 70%, 75%, 80%, 85%, 90%, 95%, 97% or 99% sequence identity to any one of SEQ ID Nos:66-69 or (ii) comprises one or more point (i.e. 1, 2, 3, 4, 5, 6, 7, 8, 9, 10 or more) mutations that add, delete or substitute any of the amino acids compared to any one of SEQ ID Nos:66-69.

53-57. (canceled)

58. A chimeric NKG2D hexamer complex comprising (i) a chimeric NKG2D protein comprising a human NKG2D extracellular domain or a variant thereof and a murine NKG2D transmembrane domain or a variant thereof, and (ii) at least one DNAX-activating protein 12 (DAP12) polypeptide or a variant thereof or at least one DNAX-activating protein 10 (DAP10) polypeptide or variant thereof.

59-61. (canceled)

62. A polypeptide for generating the chimeric NKG2D hexamer complex according to claim 58, wherein the polypeptide comprises (i) the chimeric NKG2D protein, (ii) at least one DNAX-activating protein 12 (DAP12) polypeptide or a variant thereof and/or at least one DNAX-activating protein 10 (DAP10) polypeptide or variant thereof, and (iii) one or more cleavage sites.

63. (canceled)

64. An isolated polynucleotide encoding a chimeric NKG2D protein, wherein the chimeric NKG2D protein comprises a human NKG2D extracellular domain or a variant thereof and a murine NKG2D transmembrane domain or a variant thereof.

65. (canceled)

66. The isolated polynucleotide according to claim 64, wherein the isolated polynucleotide has at least 70%, 75%, 80%, 85%, 90%, 95%, 97% or 99% sequence identity to any one of SEQ ID NOs: 70-73.

67. (canceled)

68. A host cell comprising an isolated polynucleotide according to claim 64.

69. A pharmaceutical composition comprising an immunoresponsive cell comprising a chimeric NKG2D protein: wherein the immunoresponsive cell is a T-cell, natural killer (NK) cell, macrophage or neutrophil and the chimeric NKG2D protein comprises a human NKG2D extracellular domain or a variant thereof and a murine NKG2D transmembrane domain or a variant thereof, wherein the pharmaceutical composition further comprises a Pharmaceutically or physiologically acceptable diluent or carrier.

70-74. (canceled)

75. A method of treating cancer, wherein the method comprises administering to a subject having cancer or suspected of having, an immunoresponsive cell, wherein the immunoresponsive cell comprising a chimeric NKG2D protein: wherein the immunoresponsive cell is a T-cell, natural killer (NK) cell, macrophage or neutrophil and the chimeric NKG2D protein comprises a human NKG2D extracellular domain or a variant thereof and a murine NKG2D transmembrane domain or a variant thereof.

76-77. (canceled)


SEQUENCE LISTING (78 sequences)

SEQ ID NO: 1 - protein, 232 aa, Mus musculus
MALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 2 - protein, 23 aa, Mus musculus
VVRVLAIALAIRFTLNTLMWLAI

SEQ ID NO: 3 - protein, 37 aa, Mus musculus
KISPMFVVRVLAIALAIRFTLNTLMWLAIFKETFQPV

SEQ ID NO: 4 - protein, 215 aa, Rattus norvegicus
MSKCHNYDLKPAKWDTSQEHQKQRSALPTSRPGENGIIRRRSSIEELKISPLFVVRVLVAAMTIRFTVITLTWLAVFITLLCNKEVSVSSREGYCGPCPNDWICHRNNCYQFFNENKAWNQSQASCLSQNSSLLKIYSKEEQDFLKLVKSYHWMGLVQSPANGSWQWEDGSSLSPNELTLVKTPSGTCAVYGSSFKAYTEDCSNPNTYICMKRAV

SEQ ID NO: 5 - protein, 23 aa, Rattus norvegicus
LFVVRVLVAAMTIRFTVITLTWL

SEQ ID NO: 6 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 7 - protein, 135 aa, Homo sapiens
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 8 - protein, 144 aa, Homo sapiens
IWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 9 - protein, 9 aa, Homo sapiens
IWSAVFLNS

SEQ ID NO: 10 - protein, 127 aa, Homo sapiens
PLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 11 - protein, 51 aa, Homo sapiens
MGWIRGRRSRHSWEMSEFHNYNLDLKKSDFSTRWQKQRCPVVKSKCRENAS

SEQ ID NO: 12 - protein, 50 aa, Homo sapiens
MGWIRGRRSRHSWEMSEFHNYNLDLKKSDFSTRWQKQRCPVVKSKCRENA

SEQ ID NO: 13 - protein, 53 aa, Mus musculus
MSKCHNYDLKPAKWDTSQEQQKQRLALTTSQPGENGIIRGRYPIEKLKISPMF

SEQ ID NO: 14 - protein, 47 aa, Mus musculus
MSKCHNYDLKPAKWDTSQEQQKQRLALTTSQPGENGIIRGRYPIEKL

SEQ ID NO: 15 - protein, 51 aa, Rattus norvegicus
MSKCHNYDLKPAKWDTSQEHQKQRSALPTSRPGENGIIRRRSSIEELKISP

SEQ ID NO: 16 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 17 - protein, 52 aa, Homo sapiens
YFLGRLVPRGRGAAEAATRKQRITETESPYQELQGQRSDVYSDLNTQRPYYK

SEQ ID NO: 18 - protein, 21 aa, Homo sapiens
GVLAGIVMGDLVLTVLIALAV

SEQ ID NO: 19 - protein, 40 aa, Homo sapiens
LRPVQAQAQSDCSCSTVSPGVLAGIVMGDLVLTVLIALAV

SEQ ID NO: 20 - protein, 92 aa, Homo sapiens
LRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 21 - protein, 114 aa, Mus musculus
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 22 - protein, 51 aa, Mus musculus
YSLGRLVSRGQGTAEGTRKQHIAETESPYQELQGQRPEVYSDLNTQRQYYR

SEQ ID NO: 23 - protein, 21 aa, Mus musculus
GVLAGIVLGDLVLTLLIALAV

SEQ ID NO: 24 - protein, 42 aa, Mus musculus
LSPVQAQSDTFPRCDCSSVSPGVLAGIVLGDLVLTLLIALAV

SEQ ID NO: 25 - protein, 93 aa, Mus musculus
LSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 26 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 27 - protein, 75 aa, Homo sapiens
QTTPGERSSLPAFYPGTSGSCSGCGSLSLPLLAGLVAADAVASLLIVGAVFLCARPRRSPAQEDGKVYINMPGRG

SEQ ID NO: 28 - protein, 24 aa, Homo sapiens
LCARPRRSPAQEDGKVYINMPGRG

SEQ ID NO: 29 - protein, 51 aa, Homo sapiens
QTTPGERSSLPAFYPGTSGSCSGCGSLSLPLLAGLVAADAVASLLIVGAVF

SEQ ID NO: 30 - protein, 71 aa, Homo sapiens
MIHLGHILFLLLLPVAAAQTTPGERSSLPAFYPGTSGSCSGCGSLSLPLLAGLVAADAVASLLIVGAVFLC

SEQ ID NO: 31 - protein, 53 aa, Homo sapiens
QTTPGERSSLPAFYPGTSGSCSGCGSLSLPLLAGLVAADAVASLLIVGAVFLC

SEQ ID NO: 32 - protein, 45 aa, Homo sapiens
LLAGLVAADAVASLLIVGAVFLCARPRRSPAQEDGKVYINMPGRG

SEQ ID NO: 33 - protein, 21 aa, Homo sapiens
LLAGLVAADAVASLLIVGAVF

SEQ ID NO: 34 - protein, 79 aa, Mus musculus
MDPPGYLLFLLLLPVAASQTSAGSCSGCGTLSLPLLAGLVAADAVMSLLIVGVVFVCMRPHGRPAQEDGRVYINMPGRG

SEQ ID NO: 35 - protein, 62 aa, Mus musculus
SQTSAGSCSGCGTLSLPLLAGLVAADAVMSLLIVGVVFVCMRPHGRPAQEDGRVYINMPGRG

SEQ ID NO: 36 - protein, 23 aa, Mus musculus
CMRPHGRPAQEDGRVYINMPGRG

SEQ ID NO: 37 - protein, 21 aa, Homo sapiens
MALPVTALLLPLALLLHAARP

SEQ ID NO: 38 - protein, 17 aa, Mus musculus
MDPPGYLLFLLLLPVAA

SEQ ID NO: 39 - protein, 21 aa, Mus musculus
MGALEPSWCLLFLPVLLTVGG

SEQ ID NO: 40 - protein, 21 aa, Homo sapiens
MGGLEPCSRLLLLPLLLAVSG

SEQ ID NO: 41 - protein, 6 aa, Artificial Sequence
  Tag
HHHHHH

SEQ ID NO: 42 - protein, 8 aa, Artificial Sequence
  Tag
DYKDDDDK

SEQ ID NO: 43 - protein, 15 aa, Artificial Sequence
  Tag
GLNDIFEAQKIEWHE

SEQ ID NO: 44 - protein, 14 aa, Artificial Sequence
  Tag
GKPIPNPLLGLDST

SEQ ID NO: 45 - protein, 9 aa, Artificial Sequence
  Tag
IPNPLLGLD

SEQ ID NO: 46 - protein, 10 aa, Artificial Sequence
  Tag
EQKLISEEDL

SEQ ID NO: 47 - protein, 73 aa, Homo sapiens
GVLAGIVMGDLVLTVLIALAVYFLGRLVPRGRGAAEAATRKQRITETESPYQELQGQRSDVYSDLNTQRPYYK

SEQ ID NO: 48 - protein, 42 aa, Homo sapiens
KRGRKKLLYIFKQPFMRPVQTTQEEDGCSCRFPEEEEGGCEL

SEQ ID NO: 49 - protein, 48 aa, Homo sapiens
QRRKYRSNKGESPVEPAEPCHYSCPREEEGSTIPIQEDYRKPEPACSP

SEQ ID NO: 50 - protein, 12 aa, Homo sapiens
EPKSCDKTHTCP

SEQ ID NO: 51 - protein, 45 aa, Homo sapiens
TTTPAPRPPTPAPTIASQPLSLRPEACRPAAGGAVHTRGLDFACD

SEQ ID NO: 52 - protein, 4 aa, Artificial Sequence
  linker
SGSG

SEQ ID NO: 53 - protein, 5 aa, Artificial Sequence
  linker
GSGGG

SEQ ID NO: 54 - protein, 4 aa, Artificial Sequence
  linker
GSGG

SEQ ID NO: 55 - protein, 5 aa, Artificial Sequence
  linker
GPPGS

SEQ ID NO: 56 - protein, 4 aa, Artificial Sequence
  linker
RRKR

SEQ ID NO: 57 - protein, 19 aa, Artificial Sequence
  linker
ATNFSLLKQAGDVEENPGP

SEQ ID NO: 58 - protein, 18 aa, Artificial Sequence
  linker
EGRGSLLTCGDVEENPGP

SEQ ID NO: 59 - protein, 23 aa, Artificial Sequence
  linker
SGSGATNFSLLKQAGDVEENPGP

SEQ ID NO: 60 - protein, 22 aa, Artificial Sequence
  linker
SGSGEGRGSLLTCGDVEENPGP

SEQ ID NO: 61 - protein, 27 aa, Artificial Sequence
  linker
RRKRSGSGATNFSLLKQAGDVEENPGP

SEQ ID NO: 62 - protein, 26 aa, Artificial Sequence
  linker
RRKRSGSGEGRGSLLTCGDVEENPGP

SEQ ID NO: 63 - protein, 22 aa, Artificial Sequence
  linker
VKQTLNFDLLKLAGDVESNPGP

SEQ ID NO: 64 - protein, 20 aa, Artificial Sequence
  linker
QCTNYALLKLAGDVESNPGP

SEQ ID NO: 65 - protein, 24 aa, Artificial Sequence
  linker
SGSGATNFSLLKQAGDVEENPGPM

SEQ ID NO: 66 - protein, 345 aa, Artificial Sequence
  construct
MALPVTALLLPLALLLHAARPDYKDDDDKLRPVQAQAQSDCSCSTVSPGVLAGIVMGDLVLTVLIALAVYFLGRLVPRGRGAAEAATRKQRITETESPYQELQGQRSDVYSDLNTQRPYYKRRKRSGSGATNFSLLKQAGDVEENPGPMLCARPRRSPAQEDGKVYINMPGRGKISPMFVVRVLAIALAIRFTLNTLMWLAIFKETFQPVLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 67 - protein, 316 aa, Artificial Sequence
  construct
MALPVTALLLPLALLLHAARPDYKDDDDKLRPVQAQAQSDCSCSTVSPGVLAGIVMGDLVLTVLIALAVYFLGRLVPRGRGAAEAATRKQRITETESPYQELQGQRSDVYSDLNTQRPYYKRRKRSGSGATNFSLLKQAGDVEENPGPMVVRVLAIALAIRFTLNTLMWLAIIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 68 - protein, 321 aa, Artificial Sequence
  construct
MALPVTALLLPLALLLHAARPDYKDDDDKLRPVQAQAQSDCSCSTVSPGVLAGIVMGDLVLTVLIALAVYFLGRLVPRGRGAAEAATRKQRITETESPYQELQGQRSDVYSDLNTQRPYYKRRKRSGSGATNFSLLKQAGDVEENPGPMKISPMFVVRVLAIALAIRFTLNTLMWLAIFKETFQPVLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 69 - protein, 440 aa, Artificial Sequence
  construct
MALPVTALLLPLALLLHAARPDYKDDDDKLRPVQAQAQSDCSCSTVSPGVLAGIVMGDLVLTVLIALAVYFLGRLVPRGRGAAEAATRKQRITETESPYQELQGQRSDVYSDLNTQRPYYKRRKRSGSGEGRGSLLTCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGRRKRSGSGATNFSLLKQAGDVEENPGPMKISPMFVVRVLAIALAIRFTLNTLMWLAIFKETFQPVLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 70 - DNA, 1035 bp, Artificial Sequence
  construct
atggctctgcctgtgacagctctgctgctgcctctggctctgctgctgcacgccgctagacccgattataaggacgacgacgacaagctgagacccgtgcaggcccaggcccagagcgactgcagctgcagcaccgtgagccccggcgtgctggccggcatcgtgatgggcgacctggtgctgaccgtgctcatcgcccttgccgtgtacttcctgggcagactggtccccaggggcagaggagctgccgaggccgctaccagaaagcagaggatcaccgagacagagagcccctaccaggagctgcagggccagagatccgacgtgtacagcgacctcaacacccagagaccctattacaagaggcggaagcgctccgggagtggggctaccaatttctctctcctcaagcaagccggagacgttgaggaaaaccctggacccatgctgtgcgccaggcccaggcggagccctgcccaggaggacggcaaggtgtacatcaacatgcccggccggggcaaaatatctccaatgttcgttgttcgagtccttgctatagccttggcaattcgattcacccttaacacattgatgtggcttgccattttcaaagagacgtttcagccagtactgttcaaccaggaggtgcagatccccctgaccgagagctactgcggcccctgcccaaaaaattggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 71 - DNA, 948 bp, Artificial Sequence
  construct
atggctctgcctgtgacagctctgctgctgcctctggctctgctgctgcacgccgctagacccgattataaggacgacgacgacaagctgagacccgtgcaggcccaggcccagagcgactgcagctgcagcaccgtgagccccggcgtgctggccggcatcgtgatgggcgacctggtgctgaccgtgctcatcgcccttgccgtgtacttcctgggcagactggtccccaggggcagaggagctgccgaggccgctaccagaaagcagaggatcaccgagacagagagcccctaccaggagctgcagggccagagatccgacgtgtacagcgacctcaacacccagagaccctattacaagaggcggaagcgctccgggagtggggctaccaatttctctctcctcaagcaagccggagacgttgaggaaaaccctggacccatggttgttcgagtccttgctatagccttggcaattcgattcacccttaacacattgatgtggcttgccattatctggagcgccgtgttcctgaacagcctgttcaaccaggaggtgcagatccccctgaccgagagctactgcggcccctgcccaaaaaattggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 72 - DNA, 963 bp, Artificial Sequence
  construct
atggctctgcctgtgacagctctgctgctgcctctggctctgctgctgcacgccgctagacccgattataaggacgacgacgacaagctgagacccgtgcaggcccaggcccagagcgactgcagctgcagcaccgtgagccccggcgtgctggccggcatcgtgatgggcgacctggtgctgaccgtgctcatcgcccttgccgtgtacttcctgggcagactggtccccaggggcagaggagctgccgaggccgctaccagaaagcagaggatcaccgagacagagagcccctaccaggagctgcagggccagagatccgacgtgtacagcgacctcaacacccagagaccctattacaagaggcggaagcgctccgggagtggggctaccaatttctctctcctcaagcaagccggagacgttgaggaaaaccctggacccatgaaaatatctccaatgttcgttgttcgagtccttgctatagccttggcaattcgattcacccttaacacattgatgtggcttgccattttcaaagagacgtttcagccagtactgttcaaccaggaggtgcagatccccctgaccgagagctactgcggcccctgcccaaaaaattggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 73 - DNA, 1320 bp, Artificial Sequence
  construct
atggctctgcctgtgacagctctgctgctgcctctggctctgctgctgcacgccgctagacccgattataaggacgacgacgacaagctgagacccgtgcaggcccaggcccagagcgactgcagctgcagcaccgtgagccccggcgtgctggccggcatcgtgatgggcgacctggtgctgaccgtgctcatcgcccttgccgtgtacttcctgggcagactggtccccaggggcagaggagctgccgaggccgctaccagaaagcagaggatcaccgagacagagagcccctaccaggagctgcagggccagagatccgacgtgtacagcgacctcaacacccagagaccctattacaagaggcggaagcgctccggctccggcgagggccgcggcagcctgctgacctgcggcgacgtggaagagaaccccggacccatgatccacctgggccacatcctgttcctgctgctgctgcccgtggccgctgcccaaacaacacccggcgagagatcctccttgcccgctttctatcccggaacatccggaagctgttccggatgtggatccctttctttgcctttgcttgctggattggtcgcagctgacgctgtcgcttccctccttattgtcggagctgtcttcctgtgcgccaggcccaggcggagccctgcccaggaggacggcaaggtgtacatcaacatgcccggccggggcaggcggaagcgctccgggagtggggctaccaatttctctctcctcaagcaagccggagacgttgaggaaaaccctggacccatgaaaatatctccaatgttcgttgttcgagtccttgctatagccttggcaattcgattcacccttaacacattgatgtggcttgccattttcaaagagacgtttcagccagtactgttcaaccaggaggtgcagatccccctgaccgagagctactgcggcccctgcccaaaaaattggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 74 - protein, 388 aa, Artificial Sequence
  construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 75 - DNA, 1164 bp, Artificial Sequence
  construct
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 76 - protein, 494 aa, Artificial Sequence
  construct
MALPVTALLLPLALLLHAARPDYKDDDDKQTTPGERSSLPAFYPGTSGSCSGCGSLSLPLLAGLVAADAVASLLIVGAVFYFLGRLVPRGRGAAEAATRKQRITETESPYQELQGQRSDVYSDLNTQRPYYKRRKRSGSGEGRGSLLTCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 77 - DNA, 1482 bp, Artificial Sequence
  construct
atggctctgcctgtgacagctctgctgctgcctctggctctgctgctgcacgccgctagacccgattataaggacgacgacgacaagcagaccacccctggcgagcggagcagcctgcctgccttctaccctggcaccagcggcagctgcagcggctgcggcagcctgagcctgcccctgctggctggcctggtggccgccgacgccgtggccagcctgctgatcgtgggcgccgtgttctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagcggagaaagcgctccggctccggcgagggccgcggcagcctgctgacctgcggcgacgtggaagagaaccccggacccatgatccacctgggccacatcctgttcctgctgctgctgcccgtggccgctgcccaaacaacacccggcgagagatcctccttgcccgctttctatcccggaacatccggaagctgttccggatgtggatccctttctttgcctttgcttgctggattggtcgcagctgacgctgtcgcttccctccttattgtcggagctgtcttcctgtgcgccaggcccaggcggagccctgcccaggaggacggcaaggtgtacatcaacatgcccggccggggcaggcggaagcgctccgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 78 - protein, 30 aa, Homo sapiens
QTTPGERSSLPAFYPGTSGSCSGCGSLSLP